(19) United States
(12) Patent Application Publication
(10) Pub. No.: US 2006/0205772 A1
(43) Pub. Date: Sep. 14, 2006

(54) Amide derivatives and their use as inhibitors of 11-beta-hydroxysteroid dehydrogenase type I

(76) Inventors: Gary Mark Coppola, Budd Lake, NJ (US); Robert Edson Damon, Hopkinton, MA (US); Paivi Jaana Kukkola, Asbury, NJ (US); James Lawrence Stanton, Charlestown, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/542,759

(22) PCT Filed: Jan. 23, 2004
(86) PCT No.: PCT/EP04/00571

§ 371 Date: Aug. 16, 2005

Related U.S. Application Data
(60) Provisional application No. 60/442,532, filed on Jan. 24, 2003.

(51) Int. Cl. A61K 31/4709; C07D 405/02; C07D 401/02; C07D 217/12; C07D 215/44

(52) U.S. Cl. 514/310; 546/160; 546/146

(57) ABSTRACT

Compounds of the formula (I) provide pharmacological agents which lower intracellular glucocorticoid concentrations in mammals, in particular, intracellular cortisol levels in humans. Therefore, the compounds of the instant invention improve insulin sensitivity in the muscle and the adipose tissue, and reduce lipolysis and free fatty acid production in the adipose tissue. The compounds of the invention lower hepatic glucocorticoid concentration in mammals, in particular, hepatic cortisol concentration in humans, resulting in inhibition of hepatic gluconeogenesis and lowering of plasma glucose levels. Thus, the compounds of the instant invention may be particularly useful in mammals as hypoglycemic agents for the treatment and prevention of conditions in which hyperglycemia and/or insulin resistance are implicated, such as type-2 diabetes. The compounds of the invention may also be used to treat other glucocorticoid associated disorders, such as Syndrome-X, dyslipidemia, hypertension and central obesity. The invention furthermore relates to the use of the compounds according to the invention for the preparation of medicaments, in particular of medicaments useful for the treatment and prevention of glucocorticoid associated disorders, by improving insulin sensitivity, reducing plasma glucose levels, reducing lipolysis and free fatty acid production, and by decreasing

The present invention provides amide derivatives of the formula 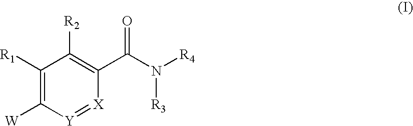
wherein
R₁ and R₂ are independently hydrogen, cyano, halo, nitro, trifluoromethyl, optionally substituted amino, alkyl, alkoxy, aryl, aralkyl, heteroaryl or heteroaralkyl; or
R₁ and R₂ combined together with the carbon atoms they are attached to form an optionally substituted 5- to 7-membered aromatic or heteroaromatic ring;
R₃ is optionally substituted lower alkyl; or
R₃ and R₂ combined together with the amide group to which R₃ is attached and the carbon atoms to which R₂ and the amide are attached form an optionally substituted 5- to 7-membered carbocyclic or heterocyclic ring;
R₄ is optionally substituted alkyl, cycloalkyl, heterocyclyl, aryl, aralkyl or heteroaralkyl; or
R₄ and R₃ taken together with the nitrogen atom to which they are attached form a 5- to 8-membered ring which may be optionally substituted or may contain another heteroatom selected from oxygen, nitrogen and sulfur; or
R₄ and R₃ taken together with the nitrogen atom to which they are attached form a 8- to 12-membered fused bicyclic ring, which may be optionally substituted or may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen, optionally substituted alkyl or aralkyl; or
   R₅ and R₁ are optionally substituted alkylene which combined together with the nitrogen atom to which R₅ is attached and the carbon atoms to which W and R₁ are attached form a 5- or 6-membered ring;
   R₆ is optionally substituted alkyl, cycloalkyl, heterocyclyl, aryl, aralkyl or heteroaralkyl;
   R₈ is optionally substituted alkyl, aralkyl or heteroaralkyl;
   R₉ is hydrogen, optionally substituted alkyl, cycloalkyl, heterocyclyl, heterocycloalkyl, aralkyl, heteroaralkyl, alkanoyl, aroyl or heteroaroyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic or heteroaromatic ring optionally substituted with alkyl, alkoxy, aryl, heteroaryl, halo, —NR₅Z, —C(O)NR₆R₇, —OR₉ or —OC(O)NR₆R₇;
X and Y are independently CH or nitrogen; or
—X═Y— is —CH₂—, oxygen, sulfur or —NR₁₀— in which R₁₀ is hydrogen or lower alkyl;
   or a pharmaceutically acceptable, salt thereof.

The compounds of the present invention provide pharmacological agents which may be employed to control local tissue concentrations of hormonally active glucocorticoids in mammals, in particular cortisol levels in humans and, therefore, may be employed for the treatment of disorders associated with elevated glucocorticoid concentrations. The compounds of the invention are inhibitors of 11β-hydroxysteroid dehydrogenase-type 1 (11β-HSD1) reductase activity. The bidirectional 11β-HSD1 enzyme acts in vivo predominantly as oxoreductase converting hormonally inactive glucocorticoids to their active 11β-hydroxy metabolites. Accordingly, the compounds of the invention lower intracellular glucocorticoid concentrations in mammals, in particular intracellular cortisol levels in humans, improving insulin sensitivity in the muscle and the adipose tissue. Furthermore, by lowering intracellular glucocorticoid concentrations in mammals, the compounds of the instant invention reduce lipolysis and free fatty acid production in the adipose tissue. The compounds of the invention also lower hepatic glucocorticoid concentration in mammals, in particular, hepatic cortisol concentration in humans, resulting in inhibition of hepatic gluconeogenesis and lowering of plasma glucose levels. The compounds of the instant invention are thus particularly useful in mammals as hypoglycemic agents for the treatment and prevention of conditions in which hyperglycemia and/or insulin resistance are implicated, such as type-2 diabetes. The compounds of the invention may also be employed to treat other glucocorticoid associated disorders, such as Syndrome-X, dyslipidemia, hypertension and central obesity. The invention furthermore relates to the use of the compounds according to the invention for the preparation of medicaments, in particular of medicaments useful for the treatment and prevention of glucocorticoid associated disorders, by improving insulin sensitivity, reducing plasma glucose levels, reducing lipolysis and free fatty acid production, and by decreasing visceral adipose tissue formation. Selective 11β-HSD1 inhibitors of the instant invention which are substantially free of undesirable side effects resulting from the inhibition of other hydroxysteroid dehydrogenases are preferred.

The present invention relates to the modulation of local tissue concentrations of hormonally active glucocorticoids, to methods by which the level of glucocorticoids may be controlled, and to useful therapeutic effects which may be obtained as a result of such control. In particular, the invention is concerned with the reduction of cortisol levels in humans. The present invention is directed to amide derivatives of formula (I), pharmaceutical compositions comprising such compounds and methods of using such compounds for the treatment of disorders associated with elevated glucocorticoid concentrations, such as type-2 diabetes, Syndrome-X, dyslipidemia, hypertension and central obesity.

Listed below are definitions of various terms used to describe the compounds of the instant invention. These definitions apply to the terms as they are used throughout the specification unless they are otherwise limited in specific instances either individually or as part of a larger group.

The term “optionally substituted alkyl” refers to unsubstituted or substituted straight or branched chain hydrocarbon groups having 1 to 20 carbon atoms, preferably 1 to 7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpenthyl, octyl and the like. Substituted alkyl groups include, but are not limited to, alkyl groups substituted by one or more of the following groups: halo, hydroxy, cycloalkyl, alkanoyl, alkoxy, alkyloxyalkoxy, alkanoyloxy, amino, alkylamino, dialkylamino, alkanoylamino, thiol, alkylthio, alkylthiono, alkylsulfonyl, arylsulfonyl, heteroarylsulfonyl, sulfonamido, nitro, cyano, carboxy, alkoxycarbonyl, aryl, aralkoxy, guanidino, heterocyclyl including indolyl, imidazolyl, furyl, thienyl, thiazolyl, pyrrolidyl, pyridyl, pyrimidyl, piperidyl, morpholinyl and the like.

The term “lower alkyl” refers to those alkyl groups as described above having 1 to 7, preferably 1 to 4 carbon atoms.

The term “halogen” or “halo” refers to fluorine, chlorine, bromine and iodine.

The term “alkenyl” refers to any of the above alkyl groups having at least 2 carbon atoms and a carbon to carbon double bond at the point of attachment. Groups having two to four carbon atoms are preferred.

The term “alkynyl” refers to any of the above alkyl groups having at least two carbon atoms and a carbon to carbon triple bond at the point of attachment. Groups having two to four carbon atoms are preferred.

The term “alkylene” refers to a straight chain bridge of 1 to 6 carbon atoms connected by single bonds (e.g., —(CH₂)ₓ— wherein x is 1 to 6), which may be substituted with 1 to 3 lower alkyl groups.

The term “cycloalkyl” refers to optionally substituted monocyclic, bicyclic or tricyclic hydrocarbon groups of 3 to 10 carbon atoms, each of which may optionally be substituted by one or more substituents such as alkyl, halo, oxo, hydroxy, alkoxy, alkanoyl, amino, alkylamino, dialkylamino, thiol, alkylthio, nitro, cyano, carboxy, carboxyalkyl, alkoxycarbonyl, alkyl- and arylsulfonyl, sulfonamido, heterocyclyl and the like.

Exemplary monocyclic hydrocarbon groups include but are not limited to cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

The term “alkoxy” refers to alkyl-O—.

The term “acyl” refers to alkanoyl, aroyl, heteroaroyl, arylalkanoyl or heteroarylalkanoyl.

The term “alkanoyl” refers to alkyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylaminothiocarbonyl” refers to alkyl-NHC(S)—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carbamoyl” refers to alkyl-NH—C(O)—, (alkyl)₂N—C(O)—, aryl-NHC(O)—, alkyl(aryl)-N—C(O)—, heteroaryl-NH—C(O)—, alkyl(heteroaryl)-N—C(O)—, aralkyl-NH—C(O)— and alkyl(aralkyl)-N—C(O)—.

The term “optionally substituted amino” refers to a primary or secondary amino group which may optionally be substituted by a substituent such as acyl, alkylsulfonyl, aryl- and heteroarylsulfonyl, aralkyl- and heteroaralkylsulfonyl, alkoxy- and cycloalkoxycarbonyl, aryloxy- and heteroaryloxycarbonyl, aralkoxy- and heteroaralkoxycarbonyl, carbamoyl, alkyl- and arylaminothiocarbonyl and the like.

The term “aryl” refers to monocyclic or bicyclic aromatic hydrocarbon groups having 6 to 12 carbon atoms in the ring-portion, such as phenyl, naphthyl, tetrahydronaphthyl, biphenyl and diphenyl groups, each of which may optionally be substituted by one to four substituents such as alkyl, halo, hydroxy, alkoxy, alkanoyl, alkanoyloxy, optionally substituted amino, thiol, alkylthio, nitro, cyano, carboxy, carboxyalkyl, alkoxycarbonyl, alkylthiono, alkyl- and arylsulfonyl, sulfonamido, heterocycloyl and the like.

The term “monocyclic aryl” refers to optionally substituted phenyl as described under aryl.

The term “aralkyl” refers to an aryl group bonded directly through an alkyl group, such as benzyl.

The term “aralkoxy” refers to an aryl group bonded directly through an alkoxy group.

The term “arylsulfonyl” refers to aryl-S(O)₂—.

The term “aroyl” refers to aryl-C(O)—.

The term “aroylamino” refers to aryl-C(O)NH—.

The term “aryloxycarbonyl” refers to aryl-O—C(O)—.

The term “heterocyclyl” or “heterocyclo” refers to an optionally substituted, fully saturated or unsaturated, aromatic or nonaromatic cyclic group, for example, which is a 4- to 7-membered monocyclic, 7- to 12-membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, dihydroquinazolinyl (such as 3,4-dihydro-4-oxo-quinazolinyl) and the like.

Exemplary tricyclic heterocyclic groups include carbazolyl, benzindolyl, phenanthrolinyl, acridinyl, phenanthridinyl, xanthenyl and the like.

The term “heterocyclyl” includes substituted heterocyclic groups. Substituted heterocyclic groups refer to heterocyclic groups substituted with 1, 2 or 3 substitutents selected from the group consisting of the following:
(a) alkyl,
(b) hydroxy (or protected hydroxy);
(c) halo;
(d) oxo, i.e., ═O;
(e) optionally substituted amino, alkylamino or dialkylamino;
(f) alkoxy;
(g) cycloalkyl;
(h) carboxy;
(i) heterocyclooxy;
(j) alkoxycarbonyl, such as unsubstituted lower alkoxycarbonyl;
(k) mercapto;
(l) nitro;
(m) cyano;
(n) sulfamoyl or sulfonamido;
(o) alkanoyloxy;
(p) aroyloxy;
(q) arylthio;
(r) aryloxy;
(s) alkylthio;
(t) formyl;
(u) carbamoyl;
(v) aralkyl; and
(w) aryl substituted with alkyl, cycloalkyl, alkoxy, hydroxy, amino, acylamino, alkylamino, dialkylamino or halo.

The term “heterocyclooxy” denotes a heterocyclic group bonded through an oxygen bridge.

The term “heteroaryl” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by, e.g., lower alkyl, lower alkoxy or halo.

The term “heteroarylsulfonyl” refers to heteroaryl-S(O)₂—.

The term “heteroaroyl” refers to heteroaroyl-C(O)—.

The term “heteroaralkyl” refer to a heteroaryl group bonded through an alkyl group.

Encompassed by the invention are prodrug derivatives, e.g., any pharmaceutically acceptable prodrug ester derivatives of the carboxylic acids of the invention which are convertible by solvolysis or under physiological conditions to the free carboxylic acids.

Examples of such carboxylic acid esters are preferably lower alkyl esters, cycloalkyl esters, lower alkenyl esters, benzyl esters, mono or disubstituted lower alkyl esters, e.g., the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxy-methyl ester, and the like conventionally used in the art.

The compounds of the invention depending on the nature of the substituents, may possess one or more asymmetric centers. The resulting diastereoisomers, enantiomers and geometric isomers are encompassed by the instant invention.

Preferred are the compounds of formula (I) wherein
R₁ and R₂ are independently hydrogen, halo, optionally substituted amino, lower alkyl or lower alkoxy; or
R₁ and R₂ combined together with the carbon atoms they are attached to form an optionally substituted 6-membered aromatic ring;
R₃ is lower alkyl; or
R₃ and R₂ combined together with the amide group to which R₃ is attached and the carbon atoms to which R₂ and the amide are attached form an optionally substituted 5- to 7-membered carbocyclic or heterocyclic ring;
R₄ is optionally substituted alkyl, cycloalkyl, heterocyclyl, aryl, aralkyl or heteroaralkyl; or
R₄ and R₃ taken together with the nitrogen atom to which they are attached form a fully saturated optionally substituted 6-membered ring; or
R₄ and R₃ taken together with the nitrogen atom to which they are attached form a fully saturated 10-membered fused bicyclic ring, which may be optionally substituted or may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or lower alkyl; or
   R₅ and R₁ are optionally substituted alkylene which combined together with the nitrogen atom to which R₅ is attached and the carbon atoms to which W and R₁ are attached form a 5-membered ring;
   R₆ is optionally substituted alkyl, cycloalkyl, aryl, heteroaryl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇;
X and Y are independently CH or nitrogen; or
—X═Y— is —CH₂—, oxygen, sulfur or —NR₁₀— in which R₁₀ is hydrogen or lower alkyl;
   or a pharmaceutically acceptable salt thereof.

Further preferred are the compounds of formula (I), designated as the A group, wherein
R₁ and R₂ are independently hydrogen, halo, optionally substituted amino, lower alkyl or lower alkoxy; or
R₁ and R₂ combined together with the carbon atoms they are attached to form an optionally substituted 6-membered aromatic ring;
R₃ is methyl or ethyl; or
R₃ and R₂ combined together with the amide group to which R₃ is attached and the carbon atoms to which R₂ and the amide are attached form a 5- to 7-membered carbocyclic ring;
R₄ is —(CHR₁₁)ₙR₁₂ in which
   n is zero or an integer from 1 to 3;
   R₁₁ is hydrogen, hydroxy or optionally substituted lower alkyl;
   R₁₂ is aryl, heterocyclyl or cycloalkyl; or
R₄ and R₃ taken together with the nitrogen atom to which they are attached form an optionally substituted decahydroquinoline or decahydroisoquinoline which may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl; or
   R₅ and R₁ are alkylene which combined together with the nitrogen atom to which R₅ is attached and the carbon atoms to which W and R₁ are attached form a 5-membered ring;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl; or
W is optionally substituted aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇;
X is CH;
Y is CH or nitrogen; or
—X═Y— is —CH₂—, oxygen, sulfur or —NR₁₀— in which R₁₀ is hydrogen or methyl;
   or a pharmaceutically acceptable salt thereof.

Preferred in the A group, designated as the B group, are the compounds wherein
R₁ and R₂ are independently hydrogen, halo, lower alkyl or lower alkoxy; or
R₁ and R₂ combined together with the carbon atoms they are attached to form an optionally substituted 6-membered aromatic ring;
R₃ is methyl or ethyl;
R₄ is —(CHR₁₁)ₙR₁₂ in which
   n is zero or an integer of 1;
   R₁₁ is hydrogen;
   R₁₂ is optionally substituted cyclohexyl; or R₁₂ is optionally substituted 1-adamantyl providing that n is an integer of 1;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl; or
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆OR₇;
X is CH;
Y is CH or nitrogen; or
—X═Y— is —CH₂—, oxygen, sulfur or —NR₁₀— in which R₁₀ is hydrogen or methyl;
   or a pharmaceutically acceptable salt thereof.

Preferred are the compounds in the B group wherein
R₁ is hydrogen;
R₂ is hydrogen, chloro or methoxy;
R₃ is methyl;
R₄ is —(CHR₁₁)ₙR₁₂ in which
   n is zero or an integer of 1;
   R₁₁ is hydrogen;
   R₁₂ is optionally substituted cyclohexyl; or R₁₂ is optionally substituted 1-adamantyl providing that n is an integer of 1;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is CH;
   or a pharmaceutically acceptable salt thereof.

Preferred are also the compounds in the B group wherein
R₁ is hydgogen;
R₂ is hydrogen or methyl;
R₃ is methyl;
R₄ is —(CHR₁₁)ₙR₁₂ in which
   n is an integer of 1;
   R₁₁ is hydrogen;
   R₁₂ is optionally substituted 1-adamantyl;
W is optionally substituted aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₉ is hydrogen, optionally substituted alkyl, aralkyl, heteroaralkyl or alkanoyl;
   Z is —C(O)R₆, —C(O)OR₆, —C(O)NR₆R₇, —C(S)NR₆R₇, —S(O)₂R₆, or —R₈ in which
      R₈ is optionally substituted alkyl, aralkyl or heteroaralkyl;
—X═Y— is —CH₂—, oxygen or —NR₁₀— in which R₁₀ is hydrogen or methyl;
   or a pharmaceutically acceptable salt thereof.

Preferred in the A group, designated as the C group, are also the compounds of the formula 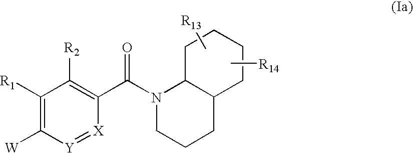
wherein
R₁ and R₂ are independently hydrogen, halo, optionally substituted amino, lower alkyl or lower alkoxy; or
R₁ and R₂ combined together with the carbon atoms to which they are attached form an optionally substituted 6-membered aromatic ring;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl; or
   R₅ and R₁ are alkylene which combined together with the nitrogen atom to which R₅ is attached and the carbon atoms to which W and R₁ are attached form a 5-membered ring;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms they are attached to form a 6-membered aromatic ring optionally substituted with alkyl, alkoxy, aryl, heteroaryl, halo, —NR₅Z, —C(O)NR₆R₇, —OR₉ or —OC(O)NR₆R₇;
X is CH;
Y is CH or nitrogen; or
—X═Y— is —CH₂—, oxygen, sulfur or —NR₁₀— in which R₁₀ is hydrogen or methyl;
R₁₃ and R₁₄ are independently hydrogen, hydroxy or optionally substituted lower alkyl;
   or a pharmaceutically acceptable salt thereof.

The compounds of formula (Ia) in the C group may contain the decahydroquinoline moiety either in the trans or the cis configuration, or a mixture of trans and cis isomers thereof. Any optical isomer or a mixture of optical isomers thereof are also encompassed by the present invention.

Preferred are the compounds of formula (Ia) wherein
R₁ is hydrogen;
R₂ is hydrogen, chloro, methoxy, ethoxy, propoxy or optionally substituted amino;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
   or a pharmaceutically acceptable salt thereof.

Preferred are also the compounds of formula (Ia) wherein
R₁ is methyl, methoxy or optionally substituted amino,
R₂ is hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
   or a pharmaceutically acceptable salt thereof.

Preferred are also the compounds of formula (Ia) wherein
R₁ and R₂ are hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is nitrogen;
R₁₃ and R₁₄ are independently hydrogen, hydroxy or optionally substituted lower alkyl;
   or a pharmaceutically acceptable salt thereof.

Preferred are also the compounds of formula (Ia) wherein
W is hydrogen;
R₂ is hydrogen;
R₁ is —NR₅Z in which Z is —C(O)R₆, —C(O)OR₆, —C(O)NR₆R₇, —C(S)NR₆R₇, —S(O)₂R₆, or —R₈ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
X is CH;
Y is CH;
R₁₃ and R₁₄ are independently hydrogen, hydroxy or optionally substituted lower alkyl;
   or a pharmaceutically acceptable salt thereof.

Preferred in the C group are also the compounds of the formula 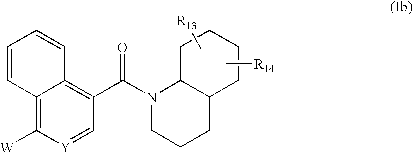
wherein
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
Y is CH;
R₁₃ and R₁₄ are independently hydrogen, hydroxy or optionally substituted lower alkyl;
   or a pharmaceutically acceptable salt thereof.

Preferred in the C group are also the compounds of the formula 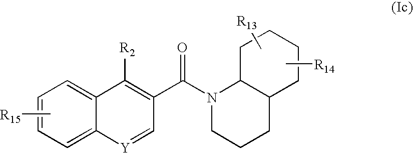
wherein
R₂ is hydrogen, halo or alkoxy;
Y is CH or nitrogen;
R₁₃ and R₁₄ are independently hydrogen, hydroxy or optionally substituted lower alkyl;
R₁₅ is hydrogen, —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
      or a pharmaceutically acceptable salt thereof.

Preferred in the C group are also the compounds of the formula 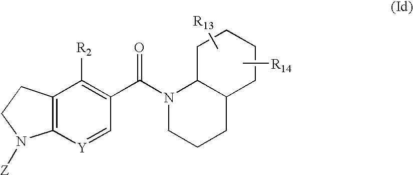
wherein
R₂ is hydrogen;
Z is —C(O)R₆, —C(O)OR₆, —C(O)NR₆R₇, —C(S)NR₆R₇, —S(O)₂R₆, or —R₈ in which
   R₆ is optionally substituted alkyl, aryl, hetroaryl, cycloalkyl, aralkyl or heteroaralkyl;
   R₇ is hydrogen or methyl;
   R₈ is hydrogen, optionally substituted alkyl, aralkyl or heteroaralkyl;
Y is CH;
R₁₃ and R₁₄ are independently hydrogen, hydroxy or optionally substituted lower alkyl;
   or a pharmaceutically acceptable salt thereof.

Preferred in the C group are also the compounds of the formula 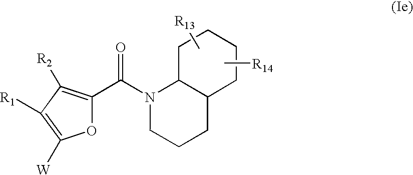
wherein
R₁ and R₂ are independently hydrogen, halo or lower alkyl;
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
   Z is —C(O)R₆, —C(O)OR₆, —C(O)NR₆R₇, —C(S)NR₆R₇, —S(O)₂R₆, or —R₈;
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
R₁₃ and R₁₄ are independently hydrogen, hydroxy or optionally substituted lower alkyl;
   or a pharmaceutically acceptable salt thereof.

Preferred in the C group are also the compounds of the formula 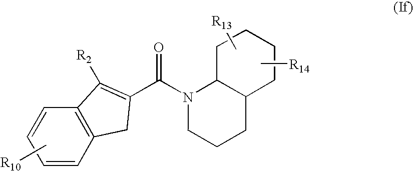
wherein
R₂ is hydrogen, halo or lower alkyl;
R₁₃ and R₁₄ are independently hydrogen, hydroxy or optionally substituted lower alkyl;
R₁₆ is hydrogen, halo, alkyl, aryl, heteroaryl or —NR₅Z in which
   Z is —C(O)R₆, —C(O)OR₆, —C(O)NR₆R₇, —C(S)NR₆R₇, —S(O)₂R₆, or —R₈;
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
      or a pharmaceutically acceptable salt thereof.

Preferred in the C group are also the compounds of the formula 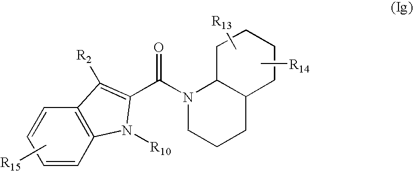
wherein
R₂ is hydrogen, halo or lower alkyl;
R₁₀ is hydrogen or methyl;
R₁₃ and R₁₄ are independently hydrogen, hydroxy or optionally substituted lower alkyl;
R₁₆ is hydrogen, halo, alkyl, aryl, heteroaryl or —NR₅Z in which
   Z is —C(O)R₆, —C(O)OR₆, —C(O)NR₆R₇, —C(S)NR₆R₇, —S(O)₂R₆, or —R₈;
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
      or a pharmaceutically acceptable salt thereof.

Preferred in the A group, designated as the D group, are also the compounds of the formula 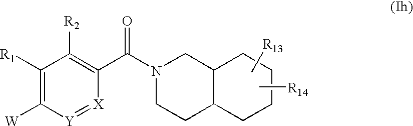
wherein
R₁ and R₂ are independently hydrogen, halo, optionally substituted amino, lower alkyl or lower alkoxy; or
R₁ and R₂ combined together form an optionally substituted 6-membered aromatic ring;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl; or
   R₅ and R₁ are alkylene which combined together with the nitrogen atom to which R₅ is attached and the carbon atoms to which W and R₁ are attached form a 5-membered ring;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl; or
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
   Z is —C(O)R₆, —C(O)OR₆, —C(O)NR₆R₇, —C(S)NR₆R₇, —S(O)₂R₆, or —R₈;
R₁₃ and R₁₄ are independently hydrogen, hydroxy or optionally substituted lower alkyl;
X is CH;
Y is CH or nitrogen; or
—X═Y— is, —CH₂—, oxygen, sulfur or —NR₁₀— in which R₁₀ is hydrogen or methyl;
   or a pharmaceutically acceptable salt thereof.

The compounds of formula (Ih) in the D group may contain the decahydroisoquinoline moiety either in the trans or the cis configuration. Any optical isomer or a mixture of optical isomers thereof are also encompassed by the present invention.

Preferred are the compounds of formula (Ih) wherein
R₁ is hydrogen;
R₂ is hydrogen, chloro, methoxy, ethoxy, propoxy or optionally substituted amino;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
   or a pharmaceutically acceptable salt thereof.

Preferred are also the compounds of formula (Ih) wherein
R₁ is methyl, methoxy or optionally substituted amino;
R₂ is hydrogen;
W is —NR₅C(O)R₆, —NR₅C(O)OR₆, —NR₅C(O)NR₆R₇, —NR₅C(S)NR₆R₇, —NR₆S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
   or a pharmaceutically acceptable salt thereof.

Preferred are also the compounds of formula (Ih) wherein
R₁ and R₂ are hydrogen;
W is —N₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
X is CH;
Y is nitrogen;
R₁₃ and R₁₄ are independently hydrogen, hydroxy or optionally substituted lower alkyl;
   or a pharmaceutically acceptable salt thereof.

Preferred in the D group are also the compounds of the formula 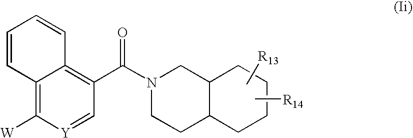
wherein
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is hydrogen, optionally substituted alkyl, aralkyl, heteroaralkyl or alkanoyl;
Y is CH;
R₁₃ and R₁₄ are independently hydrogen, hydroxy or optionally substituted lower alkyl;
   or a pharmaceutically acceptable salt thereof.

Preferred in the D group are also the compounds of the formula 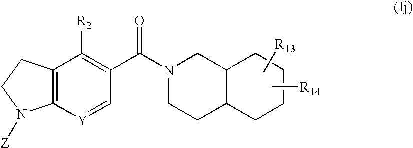
wherein
R₂ is hydrogen;
Z is —C(O)R₆, —C(O)OR₆, —C(O)NR₆R₇, —C(S)NR₆R₇, —S(O)₂R₆, or —R₈ in which
   R₆ is optionally substituted alkyl, aryl, hetroaryl, cycloalkyl, aralkyl or heteroaralkyl;
   R₇ is hydrogen or methyl,
   R₈ is hydrogen, optionally substituted alkyl, aralkyl or heteroaralkyl;
Y is CH;
R₁₃ and R₁₄ are independently hydrogen, hydroxy or optionally substituted lower alkyl;
   or a pharmaceutically acceptable salt thereof.

Preferred in the A group are also the compounds of the formula 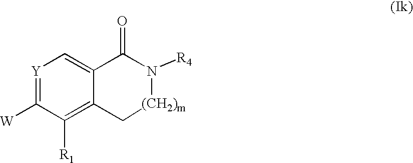
wherein
R₁ is hydrogen;
R₄ is —(CHR₁₁)ₙR₁₂ in which
   n is zero or an integer from 1 to 2;
   R₁₁ is hydrogen;
   R₁₂ is aryl, heteroaryl, heterocyclyl or cycloalkyl;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
   R₅ and R₇ are independently hydrogen or methyl;
   R₆ is optionally substituted alkyl, aryl, hetroaryl, cycloalkyl, aralkyl or heteroaralkyl;
   R₈ is optionally substituted alkyl, aralkyl or heteroaralkyl;
   R₉ is (C₁₋₆)alkyl substituted by cycloalkyl, alkoxy, cycloalkoxy, alkylthio, aryloxy, heterocyclooxy, arylthio, aryl or heteroaryl;
Y is CH;
m is zero or an integer from 1 to 2;
   or a pharmaceutically acceptable salt thereof.

Pharmaceutically acceptable salts of any acidic compounds of the invention are salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris-(hydroxymethyl)-methylammonium salts.

Similarly acid addition salts, such as of mineral acids, organic carboxylic, and organic sulfonic acids, e.g., hydrochloric acid, methanesulfonic acid, maleic acid, are possible provided a basic group, such as pyridyl, constitutes part of the structure.

Compounds of formula (I) may be prepared by reacting an activated derivative of a carboxylic acid of the formula 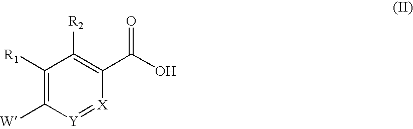
wherein R₁, R₂, X and Y have meaning as defined herein, W′ represents W as defined herein, or W′ is a group convertible to W, with an amine or an acid addition salt thereof of the formula 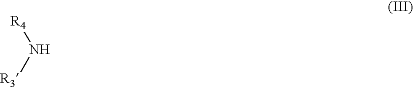
wherein R₄ has meaning as defined herein, R₃′ represents R₃ as defined herein, or R₃′ is a group convertible to R₃, to form a compound of the formula 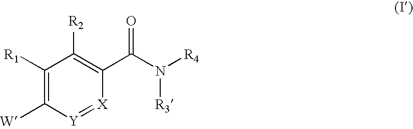
wherein R₁, R₂, R₄, X and Y have meaning as defined herein, R₃′ and W′ represent R₃ and W as defined herein, or R₃′ and W′ are groups convertible to R₃ and W, respectively. Carboxylic acids of formula (II) and amines of formula (III) may be prepared using methods described herein or modifications thereof or using methods well known in the art.

In the processes cited herein, activated derivatives of carboxylic acids, e.g., those of formula (II), include acid chlorides, bromides and fluorides, mixed anhydrides, lower alkyl esters, and activated esters thereof, and adducts formed with coupling agents such as 1-ethyl-3-(3-dimethylaminopropyl)carbodiimide hydrochloride, O-(1,2-dihydro-2-oxo-1-pyridyl)-N,N,N′,N′-tetramethyluronium tetrafluoroborate and the like. Mixed anhydrides are preferably such from pivalic acid, or lower alkyl hemiesters of carbonic acids, such as ethyl or isobutyl analogs. Activated esters include, for example, succinimido, phthalimido or 4-nitrophenyl esters. The reaction of an activated derivative of a carboxylic acid, e.g., those of formula (II), with an amine, e.g., those of formula (III), may be carried out in the presence of a base such as triethylamine, diisopropylethylamine or N-methylmorpholine in an inert solvent such as dichloromethane, N,N-dimethylformamide or tetrahydrofuran. Carboxylic acids of formula (II) can be converted to their activated derivatives using methods described herein or modifications thereof or using methods well known in the art.

Groups convertible to W include nitro, trifuoromethylsulfonate, bromine and chlorine. For example, compounds of formula (I′) in which W′ is nitro can be first reduced to the corresponding amines of the formula 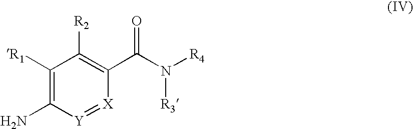
wherein R₁, R₂, R₄, X and Y have meaning as defined herein, and R₃′ represents R₃, according to methods well described in the art, e.g., with hydrogen in the presence of a catalyst such as palladium on carbon in a polar solvent such as ethyl acetate, methanol or ethanol. Compounds of formula (I′) wherein R₁, R₂, R₃′, R₄, X and Y have meaning as defined herein and W′ is nitro may be prepared as described herein or modifications thereof, or using methods well known in the art.

Alternatively, compounds of formula (IV) in which R₁, R₂, R₄, X and Y have meaning as defined herein, and R₃′ represents R₃ may be prepared by reacting compounds of formula (I′) wherein W′ is trifuoromethanesulfonate, bromine or chlorine and R₁, R₂, R₄, X and Y have meaning as defined herein, and R₃′ represents R₃, with benzophenone imine under conditions of a Buchwald condensation or using other methods well known in the art. Compounds of formula (I′) wherein R₁, R₂, R₃′, R₄, X and Y have meaning as defined herein and W′ is trifuoromethanesulfonate, bromine or chlorine may be prepared as described herein or modifications thereof, or using methods known in the art.

Compounds of formula (IV) can then be treated with a N-derivatizing agent, such as an activated derivative of a carboxylic acid, a chloroformate, a sulfonyl chloride, an isocyanate or a thioisocyanate to obtain compounds of formula (I′) in wherein R₁, R₂, R₄, X and Y have meanings as defined herein, and R₃′ represents R₃, and W′ is —NR₅C(O)R₆, —NR₅C(O)OR₆, —NR₅C(O)NR₆R₇, —NR₅C(S)NR₆R₇, —NR₅S(O)₂R₆ in which R₅, R₆ and R₇ have meanings as defined herein. The reaction to form compounds of formula (I′) may be carried out under an inert atmosphere, in the presence of a base such as triethylamine, diisopropylethylamine or N-methylmorpholine in an inert solvent or a mixture of solvents such as acetonitrile, dichloromethane, N,N-dimethylformamide or tetrahydrofuran.

Compounds of formula (I′) wherein R₁, R₂, R₄, X and Y have meanings as defined herein, and R₃′ represents R₃, and W′ is —NR₅R₈ in which R₅ and R₈ have meanings as defined herein may be obtained from compounds of formula (IV) using methods described herein or modifications thereof, or using methods well known in the art such as a reductive amination reaction.

Compounds of formula (I′) wherein R₁, R₂, R₃′, R₄, X and Y have meanings as defined herein, and W′ is —C(O)NR₆R₇ in which R₆ and R₇ have meanings as defined herein may be prepared by reacting an activated derivative of a carboxylic acid of the formula 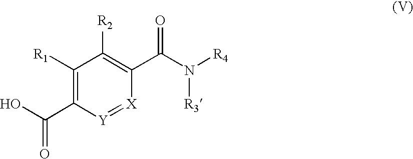
wherein R₁, R₂, R₃, R₄, X and Y have meaning as defined herein, with an amine or an acid addition salt thereof of the formula
HNR₆R₇ (VI)
wherein R₆ and R₇ have meanings as defined herein. Carboxylic acids of formula (V) and amines of formula (VI) may be prepared according to methods described herein or modifications thereof, or using methods well-known in the art.

Compounds of formula (I′) wherein W′ is hydroxy, and wherein R₁, R₂, R₃′, R₄, X and Y have meaning as defined herein may be converted to compounds of formula (I′) wherein W′ is —OR₉ or —OC(O)NR₆R₇ in which R₆, R₇ and R₉ have meanings as defined herein, according to methods described herein or modifications thereof, or using methods well known in the art, e.g., compounds of formula (I′) wherein W′ is hydroxy may be treated with an O-derivatizing agent such as an alkyl or acyl halide or an isocyanate in the presence of a base such as potassium or cesium carbonate, or an organic base such as triethylamine, diisopropylethylamine or N-methylmorpholine in an inert solvent or a mixture of solvents such as acetonitrile, dichloromethane, N,N-dimethylformamide or tetrahydrofuran. Alternatively, compounds of formula (I′) wherein W′ is hydroxy may be treated with alcohols of formula R₉OH under Mitsunobu conditions, e.g., in the presence of triphenylphoshine and diethyl azodicarboxylate in an organic solvent such as tetrahydrofuran, to afford compounds of formula (I′).

Compounds of formula (I′) wherein R₁, R₂, R₄, X, Y and W′ have meanings as defined herein, and R₃′ is a group convertible to R₃, may be converted to compounds of formula (I′) wherein R₃′ represents R₃ using methods described herein or modifications thereof, or using methods well known in the art, e.g., compounds of formula (I′) wherein R₃′ is hydrogen may be treated with an alkyl halide such as iodomethane or bromoethane in the presence of a base such as sodium hydride in an organic solvent such as N,N-dimethylformamide or tetrahydrofuran to afford compounds of formula (I′) wherein R₃′ represents R₃ such as methyl or ethyl, respectively.

Compounds of formula (Ia), (Ib), (Ic), (Id), (Ie), (If), (Ig), (Ij) and (Ik) wherein R₁, R₂, R₁₀, R₁₃, R₁₄, R₁₆, W, X, Y, Z and m, respectively, have meanings as defined herein, may be prepared similarly as described herein or modifications thereof, or using methods well known in the art.

The starting compounds and intermediates which are converted to the compounds of the invention in a manner described herein, functional groups present, such as amino, thiol, carboxyl, and hydroxy groups, are optionally protected by conventional protecting groups that are common in preparative organic chemistry. Protected amino, thiol, carboxyl, and hydroxy groups are those that can be converted under mild conditions into free amino, thiol, carboxyl and hydroxy groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxy group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, NY (1973); and Greene and Wuts, “Protective Groups in Organic Synthesis”, John Wiley and Sons, Inc., NY (1999).

The above-mentioned reactions are carried out according to standard methods, in the presence or absence of diluent, preferably such as are inert to the reagents and are solvents thereof, of catalysts, condensing or said other agents respectively and/or inert atmospheres, at low temperatures, room temperature or elevated temperatures (preferably at or near the boiling point of the solvents used), and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

The invention also relates to any novel starting materials and processes for their manufacture.

Depending on the choice of starting materials and methods, the new compounds may be in the form of one of the possible isomers or mixtures thereof, for example, as substantially pure geometric (cis or trans) isomers, optical isomers (antipodes), racemates, or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, for example by chromatography and/or fractional crystallization.

Any resulting racemates of final products or intermediates can be resolved into the optical antipodes by known methods, e.g., by separation of the diastereoisomeric salts thereof, obtained with an optically active acid or base, and liberating the optically active acidic or basic compound. The carboxylic acid intermediates can thus be resolved into their optical antipodes, e.g., by fractional crystallization of D- or L-(alpha-methylbenzylamine, cinchonidine, cinchonine, quinine, quinidine, ephedrine, dehydroabietylamine, brucine or strychnine)-salts. Racemic products can also be resolved by chiral chromatography, e.g., high pressure liquid chromatography using a chiral adsorbent.

Finally, compounds of the invention are either obtained in the free form, or as a salt thereof if salt forming groups are present.

Acidic compounds of the invention may be converted into salts with pharmaceutically acceptable bases, e.g., an aqueous alkali metal hydroxide, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

Compounds of the invention having basic groups can be converted into acid addition salts, especially pharmaceutically acceptable salts. These are formed, for example, with inorganic acids, such as mineral acids, for example sulfuric acid, a phosphoric or hydrohalic acid, or with organic carboxylic acids, such as (C₁-C₄)-alkanecarboxylic acids which, for example, are unsubstituted or substituted by halogen, for example acetic acid, such as saturated or unsaturated dicarboxylic acids, for example, oxalic, succinic, maleic or fumaric acid, such as hydroxy-carboxylic acids, for example glycolic, lactic, malic, tartaric or citric acid, such as amino acids, for example aspartic or glutamic acid, or with organic sulfonic acids, such as (C₁-C₄)-alkyl-sulfonic acids (for example methanesulfonic acid) or arylsulfonic acids which are unsubstituted or substituted (for example by halogen). Preferred are salts formed with hydrochloric acid, methanesulfonic acid and maleic acid.

In view of the close relationship between the free compounds and the compounds in the form of their salts, whenever a compound is referred to in this context, a corresponding salt is also intended, provided such is possible or appropriate under the circumstances.

The compounds, including their salts, can also be obtained in the form of their hydrates, or include other solvents used for their crystallization.

The pharmaceutical compositions according to the invention are those suitable for enteral, such as oral or rectal, transdermal and parenteral administration to mammals, including man, for the treatment of conditions associated with increased 11β-HSD1 oxoreductase activity which can lead to elevated local tissue concentrations of hormonally active glucocorticoids, such as cortisol in man. Such conditions include Syndrome-X, dyslipidemia, hypertension, central obesity, and insulin resistance and hyperglycemia in Type 2 diabetes. The said pharmaceutical compositions comprise a therapeutically effective amount of a pharmacologically active compound of the instant invention, alone or in combination with another therapeutic agent and one or more pharmaceutically acceptable carriers.

The pharmacologically active compounds of the invention may be employed in the manufacture of pharmaceutical compositions comprising a therapeutically effective amount thereof in conjunction or admixture with excipients or carriers suitable for either enteral or parenteral application. Preferred are tablets and gelatin capsules comprising the active ingredient together with a) diluents, e.g., lactose, dextrose, sucrose, mannitol, sorbitol, cellulose and/or glycine; b) lubricants, e.g., silica; talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The pharmaceutical formulations contain a therapeutically effective amount of a compound of the invention as defined above, either alone or in a combination with another therapeutic agent, e.g., each at an effective therapeutic dose as reported in the art. Such therapeutic agents include insulin, insulin derivatives and mimetics; insulin secretagogues, such as the sulfonylureas, e.g., Glipizide, glyburide and Amaryl; insulinotropic sulfonylurea receptor ligands, such as meglitinides, e.g., nateglinide and repaglinide; PPARα and/or PPARγ (peroxisome proliferator-activated receptor) ligands such as MCC-555, MK767, L-165041, GW7282 or thiazolidinediones such as rosiglitazone, pioglitazone, troglitazone; insulin sensitizers, such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441, NN-57-05445 or RXR ligands such as GW-0791, AGN-194204; sodium-dependent glucose cotransporter inhibitors, such as T-1095, glycogen phosphorylase A inhibitors, such as BAY R3401; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors such as LAF237; hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin, squalene synthase inhibitors or FXR (farnesoid X receptor) and LXR (liver X receptor) ligands, cholestyramine, fibrates, nicotinic acid and aspirin, anti-obesity agents, such as orlistat, anti-hypertensive agents, inotropic agents and hypolipidemic agents, e.g., loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolapril; inhibitors of the Na—K-ATPase membrane pump, such as digoxin; neutralendopeptidase (NEP) inhibitors; ACE/NEP inhibitors, such as omapatrilat, sampatrilat and fasidotril; angiotensin II antagonists, such as candesartan, eprosartan, irbesartan, losartan, telmisartan and valsartan, in particular valsartan; β-adrenergic receptor blockers, such as acebutolol, atenolol, betaxolol, bisoprolol, metoprolol, nadolol, propranolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil. Other specific antidiabetic compounds are described by Patel Mona (Expert Opin Investig Drugs. 2003 April; 12(4):623-33) in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the active agents identified by code numbers, generic or trade names may be taken from the actual edition of the standard compendium “The Merck Index” or from databases, e.g. Patents International (e.g. IMS World Publications). The corresponding content thereof is hereby incorporated by reference.

Thus in an additional aspect the present invention concerns a pharmaceutical composition comprising a therapeutically effective amount of a compound of the invention in combination with one or more pharmaceutically acceptable carriers.

In a further aspect the present invention concerns a pharmaceutical composition comprising a therapeutically effective amount of a compound of the invention in combination with a therapeutically effective amount of another therapeutic agent, preferably selected from anti-diabetics, hypolipidemic agents, anti-obesity agents, anti-hypertensive agents or inotropic agents, most preferably from antidiabetics or hypolipidemic agents as described above.

A pharmaceutical composition as described above for use as a medicament.

Use of a pharmaceutical composition or combination as described above for the preparation of a medicament for the treatment of conditions associated with 11β-HSD1 oxoreductase activity, preferably, impaired glucose tolerance, Type 1 or Type 2 diabetes, insulin resistance, dyslipidemia, metabolic Syndrome X and central obesity, more preferably, Type 2 diabetes, impaired glucose tolerance and central obesity.

A pharmaceutical composition as described above for the treatment of conditions associated with 11β-HSD1 oxoreductase activity, preferably, impaired glucose tolerance, Type 1 or Type 2 diabetes, insulin resistance, dyslipidemia, metabolic Syndrome X and central obesity.

A unit dosage for a mammal of about 50 to 70 kg may contain between about 1 mg and 1000 mg, advantageously between about 5 to 500 mg of the active ingredient. The therapeutically effective dosage of active compound is dependent on the species of warm-blooded animal (mammal), the body weight, age and individual condition, on the form of administration, and on the compound involved.

The compounds of the present invention are inhibitors: of 11β-HSD1, and thus may be employed for the treatment of conditions associated with increased 11β-HSD1 oxoreductase activity. Such compounds may therefore be employed for the treatment of conditions in which elevated local tissue concentrations of hormonally active glucocorticoids, such as cortisol in man, are implicated, e.g., Syndrome-X, dyslipidemia, hypertension, central obesity, and insulin resistance and hyperglycemia in Type 2 diabetes.

Thus, in an additional embodiment, the present invention relates to:

A compound of the invention for use as a medicament.

The use of a compound of the invention for the preparation of a pharmaceutical composition for the prevention and/or treatment of conditions associated with increased 11β-HSD1 oxoreductase activity.

A pharmaceutical composition, for use in conditions associated with 11β-HSD1 oxoreductase activity comprising a compound of formula (I) in free form or pharmaceutically acceptable salt form in association with a pharmaceutically acceptable diluent or carrier therefore.

A method for the prevention and/or treatment of conditions associated with 11β-HSD1 oxoreductase activity, which comprises administering a therapeutically effective amount of a compound of the present invention.

In accordance with the foregoing the present invention provides in a yet further aspect:

A therapeutic combination, e.g. a kit, kit of parts e.g. for use in any method as defined herein, comprising a compound of formula (I), in free form or in pharmaceutically acceptable salt form, to be used concomitantly or in sequence with at least one pharmaceutical composition comprising at least another therapeutic agent, preferably selected from antidiabetics, hypolipidemic agents, anti-obesity agents, anti-hypertensive agents or inotropic agents. The kit may comprise instructions for its administration.

A kit of parts comprising

(i) a pharmaceutical composition of the invention, (ii) a pharmaceutical composition comprising a compound selected from an antidiabetic, anti-obesity agent, anti-hypertensive agent, inotropic agent or hypolipidemic agent, or a pharmaceutically acceptable salt thereof, in the form of two separate units of the components (i) to (ii).

A method as defined above comprising co-administration, e.g. concomitantly or in sequence, of a therapeutically effective amount of a compound of formula (I) in free form or in pharmaceutically acceptable salt form, and a second drug substance, said second drug substance being a antidiabetic, anti-obesity agent, anti-hypertensive agent, inotropic agent or hypolipidemic agent, e.g. as indicated above.

Preferably, a compound of the invention is administered to a mammal in need thereof.

Preferably, a compound of the invention is used for the treatment of a disease which responds to inhibition of 11β-HSD1 oxoreductase activity.

Preferably, the conditions associated with increased 11β-HSD1 oxoreductase activity are selected from impaired glucose tolerance, Type 1 or Type 2 diabetes, insulin resistance, dyslipidemia, metabolic Syndrome X and central obesity, most preferably Type 2 diabetes, impaired glucose tolerance and central obesity.

A method or use according to the invention which comprises administering said compound in combination with a therapeutically effective amount of an antidiabetic agent, anti-obesity agent, anti-hypertensive agent, inotropic agent or hypolipidemic agent.

A method or use according to the invention which comprises administering said compound in the form of a pharmaceutical composition as described herein.

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administraton, between about 1 and 500 mg/kg, preferably between about 5 and 100 mg/kg.

The activity of a compound according to the present invention can be assessed by the following methods or methods well described in the art:

The in vitro inhibition of human recombinant 11β-HSD1 is determined as follows:

Recombinant human 11β-HSD1 is expressed in yeast Pichia pastoris. Cultures are grown at 30° C. for 3 days in the presence of methanol to induce enzyme expression. The microsomal fraction overexpressing 11β-HSD1 is prepared from the cell homogenate and used as the enzyme source for primary screening. A test compound at the desired concentration is preincubated for 10 min at RT with 3 μg of the microsomal protein in 50 mM sodium phosphate, pH 7.5, in a total volume of 80 μL. The enzyme reaction is initiated by adding 20 μL of a mixture containing 5 mM NADPH, 500 nM cortisone, and 80,000 dpm of [³H]cortisone in the same buffer and is terminated by ethyl acetate after incubation for 90 min at 37° C. The production of [³H]cortisol is quantitated upon separation from [³H]cortisone by a C₁₈ column on HPLC equipped with a radioactivity detector. Glycerrhetinic acid, a known inhibitor of 11β-HSD1, is used as a standard.

The in vitro inhibition of human 11β-HSD2 is determined as follows:

The SW-620 human colon carcinoma cell line is obtained from the American Type Culture Collection (ATCC). Cells are plated at a density of 8-10×10⁴ cells/cm² in DMEM/F12 containing 5% BCS, 100 U/mL penicillin, 100 μg/mL streptomycin and 0.25 μg/L amphotericin B. Cultures are grown to 80-90% confluence in a humidified, atmosphere of 5% CO₂ at 37° C. The medium is changed to serum-free, phenol red-free DMEM/F12 at 24 h before harvesting the cells.

After 24 h in serum-free medium, cultured SW-620 cells are rinsed and scraped in Kreb's-Ringer buffer, pH 7.4, containing 1 mM EDTA, 2 μg/mL aprotinin, 10 μM leupeptin and 1 μM pepstatin. After sonication (30 seconds) and low speed centrifugation (2,000 rpm, 5 min) to remove cellular debris, the supernatant is collected and used to determine enzyme activity and protein concentration (BCA, Pierce, Rockford, Ill.).

Dehydrogenase activity is quantified by measuring the conversion of [³H]cortisol to [³H]cortisone using lysates of SW-620 cells as the enzyme source. The assay is performed in tubes containing Kreb's-Ringer buffer pH 7.4, with 0.20 mM NAD and 200,000 dpm of [³H]cortisol and a test compound in a total volume of 1 mL. The tubes are preincubated for 10 min at 37° C. before adding 200 μg of cell lysates to start the reaction. After incubation for 1 h at 37° C. in a shaking water bath, the mixture is extracted with 2 volumes of ethyl acetate and centrifuged for 10 min at 2,000 rpm. The organic layer is collected, dried under vacuum and resuspended in methanol. The dissolved residues are quantitatively transferred to thin layer plates and developed in chloroform-methanol (90:10). Unlabeled cortisol and cortisone were used as reference markers. The TLC plates are scanned on a Bioscan radioimaging detector (Bioscan, Washington, D.C.), and the fractional conversion of cortisol to cortisone is calculated. Enzyme activity is expressed as pmoles of product formed per mg protein per hour. Carbenoxolone and glycyrrhetinic acid are used as standards.

The inhibition of cellular 11β-HSD1 activity in primary rat hepatocytes is determined as follows:

Male Sprague-Dawley rats weighing 180-200 g are anesthetized with sodium pentobarbital (65 mg/kg). The liver is perfused in situ with calcium-free Earl's Balanced Salt Solution (EBSS) followed by EBSS containing 100-150 U/mL of collagenase, 1.8 mM CaCl₂ and 10 mM HEPES, pH 7.4. The perfused liver is removed and aseptically placed in warm William's Medium E containing 10% BCS. After decapsulation, the organ is transferred to fresh medium and gently shaken to facilitate tissue dissociation and cell release. Hepatocytes are separated from nonparenchymal and dead cells by repeated low speed centrifugation. Cell viability is determined by trypan blue exclusion.

Hepatocytes are plated on collagen coated dishes at a density of 1×10⁵ cells/cm² in William's medium E containing 10% BCS, 100 U/mL penicillin, 100 μg/mL streptomycin, 0.25 μg/mL amphotericin B, 2 mM L-glutamine, 10 mM HEPES, 100 nM insulin and 1 nM dexamethasone. After 1 h the medium is changed to serum-free William's medium E supplemented as described above. Thereafter, the medium is replaced every 24 h. The cultures are maintained in a humidified atmosphere of 5% CO₂ at 37° C.

Enzyme activity is measured in the medium of primary cultures of rat hepatocytes 48 h after plating the cells. The medium is aspirated and replaced with serum-free William's medium E containing 2 nM [³H]11-dehydrocorticosterone and a test compound and is incubated for 2 h. An aliquot of culture medium is removed at the end of the incubation and the mixture is extracted with 2 volumes of ethyl acetate, dried under vacuum and resuspended in methanol. The dissolved residues are quantitatively transferred to thin layer plates and developed in chloroform-methanol (90:10). The TLC plates are scanned on a Bioscan imaging detector and the fractional conversion of 11-dehydrocorticosterone to corticosterone is calculated. The cell layer is rinsed with cold phosphate-buffered saline and dissolved in 0.1 N NaOH/5% SDS for the determination of cellular protein (BCA, Pierce, Rockford, Ill.). Enzyme activity is expressed as pmoles of product formed per mg protein per hour.

Inhibition of corticosterone production in adrenalectomized (ADX) mice is determined as follows:

Bilateral adrenalectomy is performed in male mice of the CD1 strain (6 to 8 weeks of age, 25-30 g body weight) through a lumbar laparotomy. After 10 days the animals are fasted for 24 h. Compounds are administered orally at 25 mg/kg each at 2 and 4 h before sacrifice. A second group of animals receives carbenoxolone at the same dose, and a third group receives the vehicle (cornstarch). Homogenized liver samples are used to measure corticosterone concentration which is determined by radioimmunoassay and is expressed as pg of corticosterone per mg of liver protein.

Illustrative of the invention are the compounds of the following examples:

| | | | cellular | |
|---|---|---|---|---|
| | | 11β-HSD2 | 11β-HSD1 | ADX mice |
| | 11β-HSD1 | % inhibition | % inhibition | % change in |
| Example | IC₅₀ (nM) | @ 10 μM | @ 1 μM | corticosterone |
| 3-11 | 1000 | 26 | 80 | −69 |
| 8-6 | 6.5 | 11 | 54 | −57 |
| 8-9 | 543 | 30 | 75 | −53 |
| 11-13 | 563 | 2 | 90 | −73 |
| 13-4 | 42 | 44 | 53 | −70 |
| 23-47 | 2120 | 49 | 71 | −61 |
| 33-21 | 262 | 45 | 84 | −71 |
| 35-15 | 7.7 | 34 | 76 | −67 |
| 38 | 180 | 10 | 49 | −62 |
| 48-37 | 770 | 29 | 75 | −69 |
| 48-65 | 560 | 30 | 67 | −73 |

The following Examples are intended to illustrate the invention and are not to be construed as being limitations thereon. Temperatures are given in degrees Centrigrade. If not mentioned otherwise, all evaporations are performed under reduced pressure, preferably between about 15 and 100 mmHg (=20-133 mbar). The structure of final products, intermediates and starting materials is confirmed by standard analytical methods, e.g., microanalysis, melting point (mp) and spectroscopic characteristics (e.g., MS, IR, NMR). Abbreviations used are those conventional in the art.

### EXAMPLE 1

### N-Cyclohexylmethyl-4-fluorobenzoylamino-N-methylbenzamide

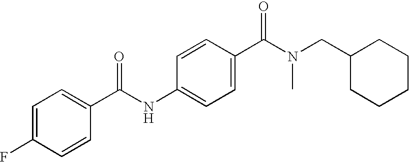

### A. 4-(4-Fluorobenzoylamino)benzoic acid ethyl ester

To a solution of 5.06 g (30 mmol) of ethyl-4-aminobenzoate and 3.92 g (30 mmol) of N,N-diisopropylethylamine in 150 mL of 1,2-dichloroethane is added 4.85 g (30 mmol) of 4-fluorobenzoylchloride dropwise while stirring under nitrogen at room temperature (RT). The mixture is stirred for 20 h further at RT. The precipitate which formed is collected by filtration to give 4-(4-fluorobenzoylamino)benzoic acid ethyl ester. The filtrate is concentrated and the concentrate is suspended in water and stirred until crystallization occurs. The precipitate is collected by filtration, washed with water, and dried to give a second crop of product: m.p. 172-174° C.; IR (KBr) 1706, 1655; API-MS 288 [M+1]⁺; NMR (DMSO-d₆) 1.32 (t, 3H), 4.30 (q, 2H), 7.39 (dd, 2H), 8.00 (m, 4H), 8.06 (m, 2H), 10.60 (s, 1H).

### B. 4-(4-Fluorobenzoylamino)benzoic acid

To a suspension of 1.43 g (5 mmol) of the title A compound, 4-(4-fluorobenzoylamino)benzoic acid ethyl ester, 50 mL of water, and 50 mL of EtOH is added 5.5 mL (5.5 mmol) of 1 N aqueous sodium hydroxide (NaOH) dropwise while stirring at RT under nitrogen. The mixture is refluxed for 1 h, then cooled to RT and the solvent is removed until crystallization begins. The concentrate is extracted with 50 mL of diethyl ether and the aqueous layer is acidified by the addition of 5.5 mL of 1 N aqueous hydrochloric acid (HCl). The precipitate is collected by vacuum filtration, washed with water, and dried to give 4-[(4-fluorobenzoyl)amino]benzoic acid: m.p. >300° C.; IR (KBr) 1679, 1649; NMR (DMSO-d₆) 7.40 (dd, 2H), 7.93 (m, 4H), 8.06 (m, 2H), 10.66 (s, 1H), 12.76 (broad s, 1H); API-MS 260.0 [M+1]⁺, 258.0 [M−1]⁻.

### C. 4-(4-Fluorobenzoylamino)benzoyl chloride

To a suspension of 3.11 g (12 mmol) of the title B compound, 4-(4-fluorobenzoylamino)benzoic acid and 300 mL of anhydrous toluene is added 0.19 g (2.4 mmol) of anhydrous pyridine followed by 2.07 g (17.5 mmol) of thionyl chloride while stirring at RT under nitrogen. The mixture is stirred at 55° C. for 21 h. After cooling to 0° C. the precipitate is collected by vacuum filtration, washed with toluene and cyclohexane, and dried to give 4-(4-fluorobenzoylamino)benzoyl chloride: IR (KBr) 1773, 1748, 1675; NMR (DMSO-d₆) 7.39 (dd, 2H), 7.93 (m, 4H), 8.07 (m, 2H), 10.60 (s, 1H).

### D. N-Cyclohexylmethyl-4-fluorobenzoylamino-N-methylbenzamide

To a solution of 0.065 g (0.51 mmol) of N-methylcyclohexyl-methylamine, 0.067 g (0.51 mmol) of N,N-diisopropylethylamine and 20 mL of 1,2-dichloroethane is added 0.14 g (0.51 mmol) of the title C compound, 4-[(4-fluorobenzoyl)amino]-benzoyl chloride while stirring at RT under nitrogen. The mixture is stirred for 21 h at RT. The mixture is filtered and the filtrate is concentrated to an oil. The oil is suspended and stirred in 20 mL of water until crystallization occurs. The precipitate is collected by filtration and dried to give N-cyclohexylmethyl-4-fluorobenzoylamino-N-methylbenzamide: m.p. 158-160° C.; IR (KBr) 1674.6, 1604.1; API-MS 369 [M+1]⁺, 367 [M−1]⁻; NMR (DMSO-d₆)-0.68 (m, 1H), 1.19 (m, 4H); 1.68 (m, 6H), 3.04 (d, 3H), 2.25 (d, 1H), 3.41 (d; 1H), 7.25 (t, 2H), 7.41 (t, 2H), 7.81 (d, 2H), 8.01 (m, 2H).

Alternatively, N-cyclohexylmethyl-4-fluorobenzoylamino-N-methylbenzamide may be prepared as follows:

### A′. N-Cyclohexylmethyl-N-4-nitrobenzamide

A solution of 4-nitrobenzoyl chloride (8.00 g, 43.08 mmol) in 50 mL tetrahydrofuran (THF) is cooled to 0° C. and treated sequentially with cyclohexylmethylamine (7.3 my, 5.6.00 mmol) and N-methylmorpholine (NMM, 7.1 mL, 64.62 mmol). The suspension is stirred at RT for 17 h. The product, N-cyclohexylmethyl-4-nitrobenzamide is collected by vacuum filtration to afford an off-white solid: NMR (DMSO-d₆) 0.86-1.08 (m, 2H), 1.12-1.26 (m, 3H), 1.51-1.74 (m, 6H), 3.13 (t, 2H, J=6.4), 8.17 (d, 2H, J=73.8), 8.20 (d, 2H, J=73.8), 8.77 (br t, 1H, J=5.3).

### B′. N-cyclohexylmethyl-N-methyl-4-nitrobenzamide

A solution of the title A′ compound, N-cyclohexylmethyl-4-nitrobenzamide (2.62 g, 10.0 mmol) in 50 mL of THF is treated with sodium hydride (720 mg, 18.0 mmol). After stirring at RT for 20 min, iodomethane (1.87 mL, 30.0 mmol) is added, and the reaction is stirred at RT for 16 h. The reaction is quenched with water, and the product is taken up in ethyl acetate (EtOAc). The organic layer is washed sequentially with saturated aqueous lithium chloride and brine, dried over anhydrous sodium sulfate (Na₂SO₄), and concentrated. The residue is suspended in hexanes to solidify the product. The product is collected by vacuum filtration to afford N-cyclohexylmethyl-N-methyl-4-nitrobenzamide as a yellow solid: NMR (CDCl₃) 0.55-0.67 (m, 1H), 1.01-1.33 (m, 4H), 1.56-1.77 (m, 6H), 2.91 (s, 1H), 3.04-3.09 (m, 3H), 3.42 (d, 1H, J=7.2), 7.52-7.58 (m, 2H), 8.28 (d, 2H, J=8.7).

### C′. 4-Amino-N-cyclohexylmethyl-N-methylbenzamide

A mixture of the title B′ compound, N-cyclohexylmethyl-N-methyl-4-nitrobenzamide (2.20 g, 7.91 mmol) and 10% palladium-on-carbon (Pd/C, 330 mg) in 100 mL EtOH is hydrogenated under 1 atm hydrogen at RT for 16 h. The catalyst is removed by vacuum filtration through celite. The residue is passed through a silica gel column (EtOAc) to afford 4-amino-N-cyclohexylmethyl-N-methylbenzamide as a thick, yellow oil: NMR (DMSO-d₆) 0.68-0.90 (br m, 2H), 1.07-1.27 (m, 3H), 1.50-1.70 (br m, 6H), 2.90 (s, 3H), 3.22 (d, 2H, J=7.2), 5.45 (s, 1H), 6.52 (, 2H, J=8.7), 7.08 (d, 2H, J=7.9).

### D′. N-Cyclohexylmethyl-4-fluorobenzoylamino-N-methylbenzamide

Under multiparallel solution phase synthesis conditions, solutions of NMM (2.0 M in THF, 126 μL, 0.225 mmol) and 4-fluorobenzoyl chloride (1.0 M in THF, 195 μL, 0.195 mmol) are dispensed sequentially into a vial containing a solution of the title C′ compound, 4-amino-N-cyclohexylmethyl-N-methylbenzamide in N,N-dimethylformamide (DMF, 0.30 M, 4500 μL, 0.15 mmol). The vial is shaken at RT for 5 h, then PS Trisamine (Argoscoop set at 0.5, Argonaut Technologies, Inc.) is added to the vial. The vial is shaken at RT for additional 16 h. The reaction mixture is filtered, acidified with 50 μL trifluoroacetic acid (TFA) and purified by HPLC to afford N-cyclohexylmethyl-4-fluorobenzoylamino-N-methylbenzamide: API-MS 369 [M+1]⁺.

### EXAMPLE 2

The following compounds are prepared analogously to Example 1 by treating the title C′ compound in Example 1 with the appropriate activated derivative of a carboxylic acid.

| Compd | Structure | MS [m/z] |
|---|---|---|
| 2-1 | 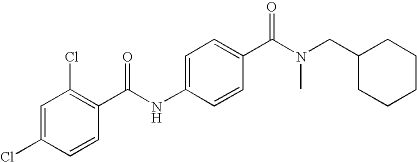 | [M + 1]⁺ 419 |
| 2-2 | 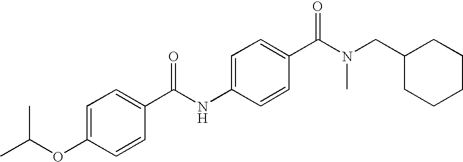 | [M + 1]⁺ 409 |
| 2-3 | 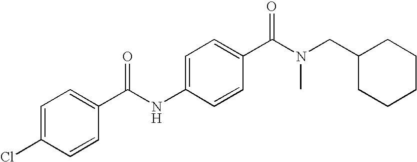 | [M + 1]⁺ 385 |
| 2-4 | 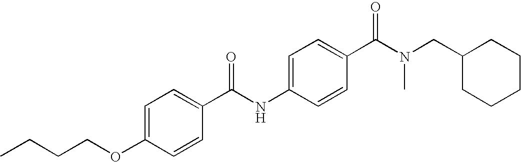 | [M + 1]⁺ 423 |
| 2-5 | 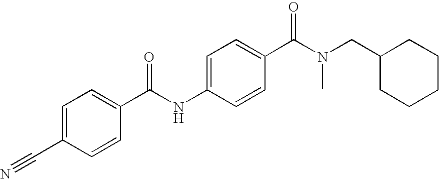 | [M + 1]⁺ 376 |
| 2-6 | 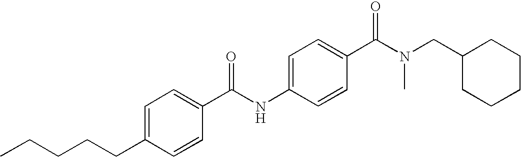 | [M + 1]⁺ 421 |
| 2-7 | 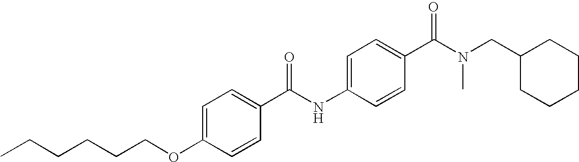 | [M + 1]⁺ 451 |
| 2-8 | 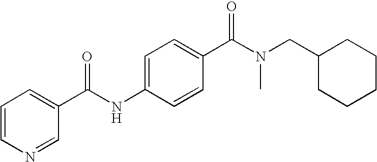 | [M + 1]⁺ 352 |
| 2-9 | 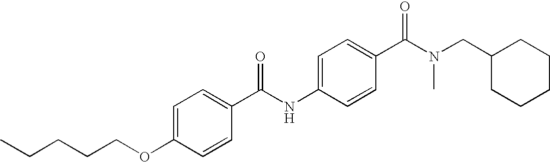 | [M + 1]⁺ 437 |
| 2-10 | 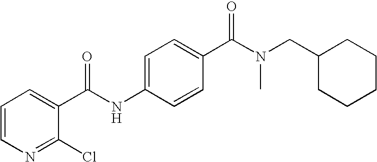 | [M + 1]⁺ 386 |
| 2-11 | 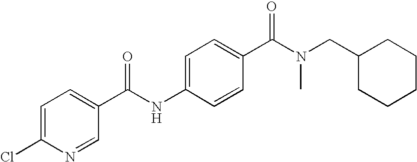 | [M + 1]⁺ 386 |
| 2-12 | 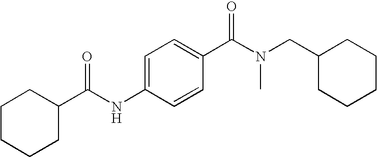 | [M + 1]⁺ 357 |
| 2-13 | 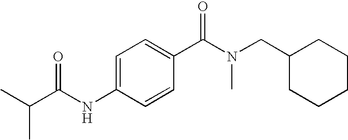 | [M + 1]⁺ 317 |
| 2-14 | 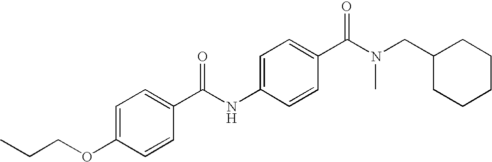 | [M + 1]⁺ 409 |
| 2-15 | 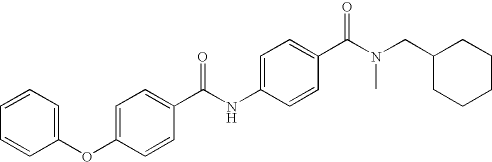 | [M + 1]⁺ 443 |
| 2-16 | 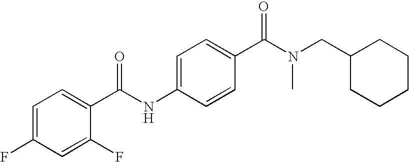 | [M + 1]⁺ 387 |
| 2-17 | 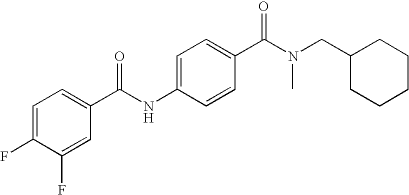 | [M + 1]⁺ 387 |
| 2-18 | 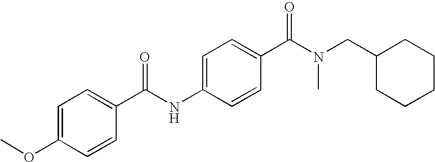 | [M + 1]⁺ 381 |

### EXAMPLE 3

The following compounds are prepared analogously to Examples 1 and 2 starting from 2-chloro-4-nitrobenzoyl chloride and cyclohexylmethylamine and treating the intermediate 4-amino-2-chlorobenzamide derivative analogous to the title C′ compound in Example 1 with the appropriate N-derivatizing agent, such as an activated derivative of a carboxylic acid, a chloroformate or an isocyanate.

| Compd | Structure | MS [m/z] |
|---|---|---|
| 3-1 | 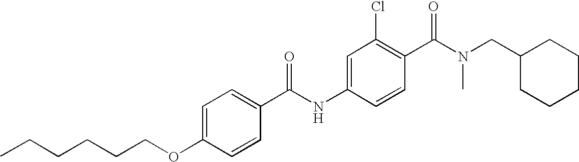 | [M + 1]⁺ 485 |
| 3-2 | 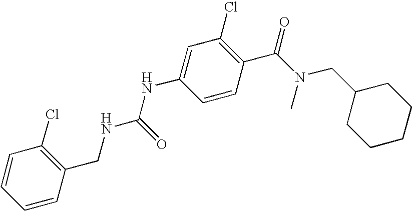 | [M + 1]⁺ 448 |
| 3-3 | 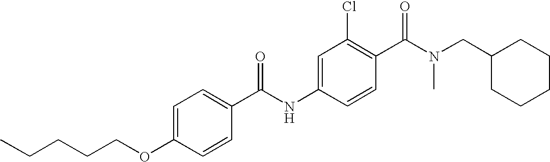 | [M + 1]⁺ 471 |
| 3-4 | 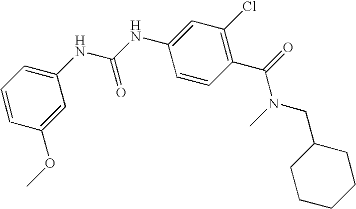 | [M + 1]⁺ 431 |
| 3-5 | 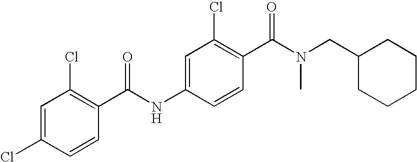 | [M + 1]⁺ 455 |
| 3-6 | 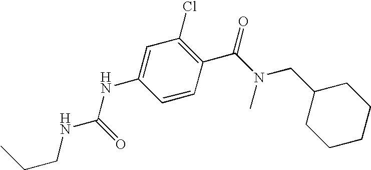 | [M + 1]⁺ 367 |
| 3-7 | 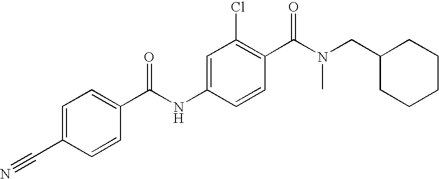 | [M + 1]⁺ 410 |
| 3-8 | 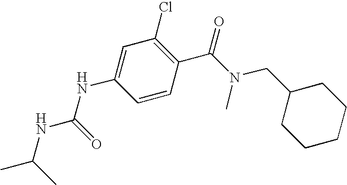 | [M + 1]⁺ 367 |
| 3-9 | 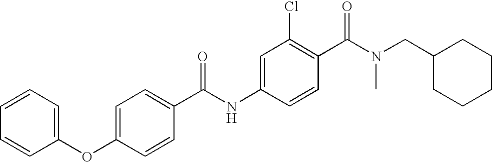 | [M + 1]⁺ 477 |
| 3-10 | 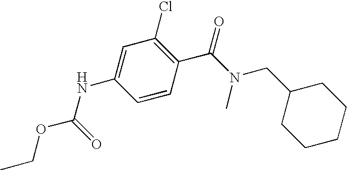 | [M + 1]⁺ 354 |
| 3-11 | 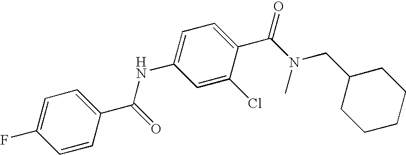 | [M + 1]⁺ 403 |
| 3-12 | 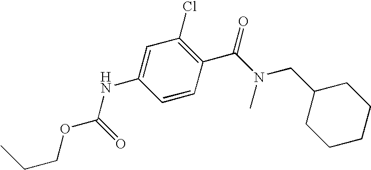 | [M + 1]⁺ 368 |
| 3-13 | 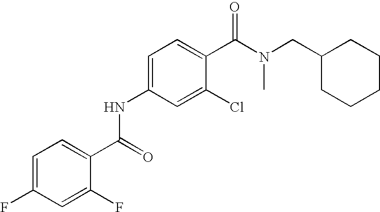 | [M + 1]⁺ 421 |
| 3-14 | 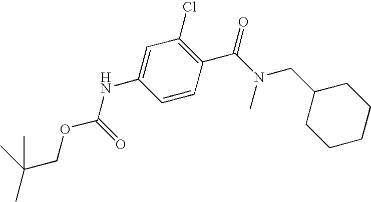 | [M + 1]⁺ 396 |
| 3-15 | 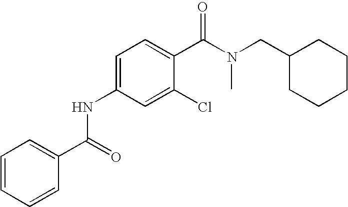 | [M + 1]⁺ 385 |
| 3-16 | 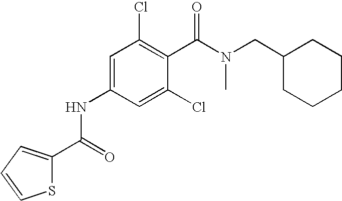 | [M + 1]⁺ 391 |
| 3-17 | 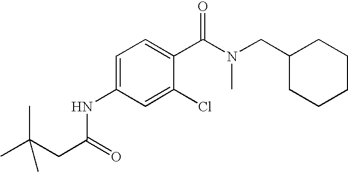 | [M + 1]⁺ 380 |
| 3-18 | 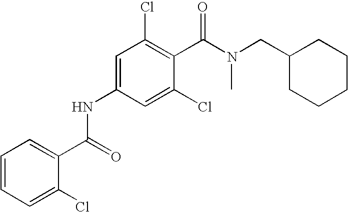 | [M + 1]⁺ 381 |

### EXAMPLE 4

The following compounds are prepared analogously to Examples 1 and 2 starting from 2-methoxy-4-nitrobenzoyl chloride and cyclohexylmethylamine and treating the intermediate 4-amino-2-methoxybenzamide derivative analogous to the title C′ compound in Example 1 with the appropriate N-derivatizing agent, such as an activated derivative of a carboxylic acid.

| Compd | Structure | MS [m/z] |
|---|---|---|
| 4-1 | 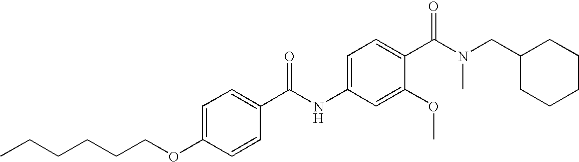 | [M + 1]⁺ 481 |
| 4-2 | 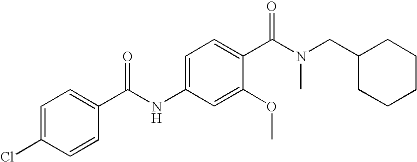 | [M + 1]⁺ 415 |
| 4-3 | 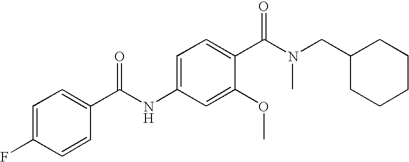 | [M + 1]⁺ 399 |
| 4-4 | 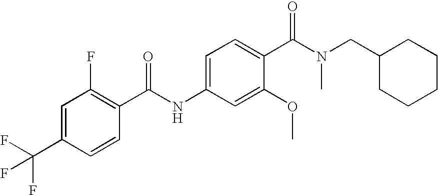 | [M + 1]⁺ 467 |
| 4-5 | 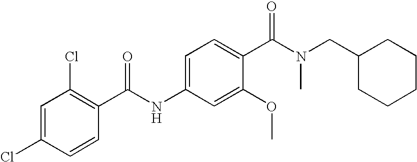 | [M + 1]⁺ 449 |
| 4-6 | 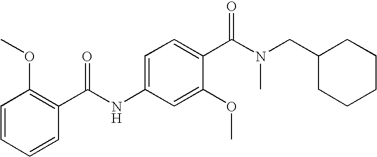 | [M + 1]⁺ 411 |
| 4-7 | 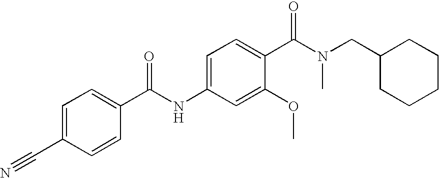 | [M + 1]⁺ 406 |
| 4-8 | 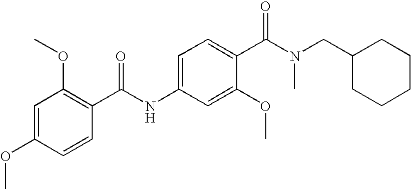 | [M + 1]⁺ 441 |
| 4-9 | 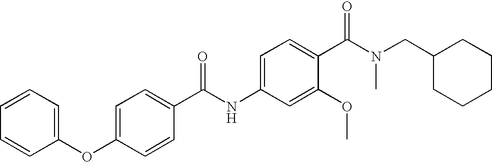 | [M + 1]⁺ 473 |
| 4-10 | 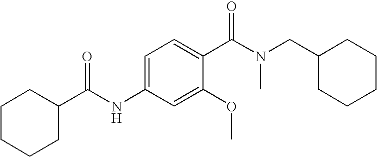 | [M + 1]⁺ 386 |
| 4-11 | 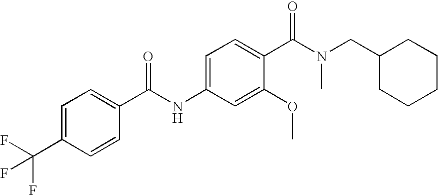 | [M + 1]⁺ 449 |
| 4-12 | 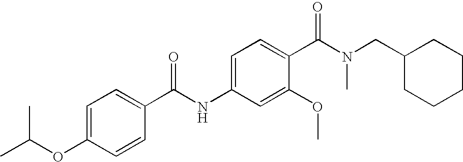 | [M + 1]⁺ 387 |

### EXAMPLE 5

### 4-(4-Chlorobenzylamino)-N-cyclohexylmethyl-N-methylbenzamide

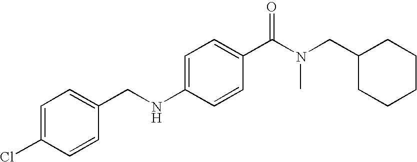

### A. {4-[(Cyclohexylmethyl)methylcarbamoyl]phenyl}carbamic acid allyl ester

A solution of the title C′ compound in Example 1, 4-amino-N-cyclohexylmethyl-N-methylbenzamide (500 mg, 2.03 mmol) in 20 mL THF at 0° C. is treated sequentially with NMM (0.29 mL, 2.64 mmol) and allyl chlorformate (0.24 mL, 2.24 mmol). The reaction is stirred at 0° C. for 4 h, then partitioned between EtOAc and water. The organic layer is washed with brine, dried over anhydrous Na₂SO₄, and concentrated to afford {4-[(cyclohexylmethyl)methylcarbamoyl]phenyl}carbamic acid allyl ester as a yellow oil: NMR (CDCl₃) 0.55-0.72 (br m, 2H), 1.00-1.32 (br m, 4H), 1.55-1.80 (br m, 6H), 2.96-3.03 (m, 3H), 3.10-3.45 (br m, 2H), 4.68 (d, 2H, J=5.9), 5.26-5.40 (m, 2H), 5.91-6.04 (m, 1H), 6.83 (s, 1H), 7.35-7.43 (m, 4H).

### B. 4-(4-Chlorobenzylamino)-N-cyclohexylmethyl-N-methylbenzamide

The sodium salt of the title A compound, {4-[(cyclohexylmethyl)methylcarbamoyl]-phenyl}carbamic acid allyl ester is prepared in a 3 mL volumetric flask by dissolving the title A compound (330 mg, 1.00 mmol) in 2 mL of THF, adding NaH (60% suspension in mineral oil, 44 mg, 1.1 mmol), and diluting the mixture to 3 mL of total volume with DMF. The mixture is shaken for 10 min and used immediately.

Under multiparallel solution phase synthesis conditions, solutions of the sodium salt (0.33 M, 450 μL, 0.15 mmol) and 4-chlorobenzyl bromide (2.0 M in THF, 98 μL, 0.195 mmol) are dispensed sequentially into a vial. The vial is shaken at RT for 16 h, then morpholine (40 μL, 0.45 mmol) and solutions of 3,3′,3″-phoshinidynetris(benzenesulfonic acid) trisodium salt (0.15 M in water, 200 μL, 0.03 mmol), and palladium(II) acetate in acetonitrile (0.10 M, 150 μL, 0.15 mmol) are dispensed into the vial, and the vial is shaken for 30 min. The reaction mixture is filtered, acidified with 50 μL TFA, and purified by HPLC to afford 4-(4-chlorobenzylamino)-N-cyclohexylmethyl-N-methylbenzamide: API-MS 371 [M+1]⁺.

### EXAMPLE 6

The following compounds are prepared analogously to Example 5 by converting the title A compound in Example 5 to its sodium salt, treating the sodium salt with the appropriate alkylating agent followed by deallylation.

| Compd | Structure | MS [m/z] |
|---|---|---|
| 6-1 | 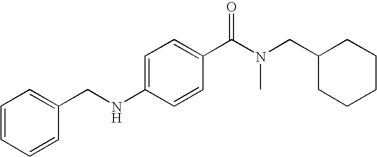 | [M + 1]⁺ 337 |
| 6-2 | 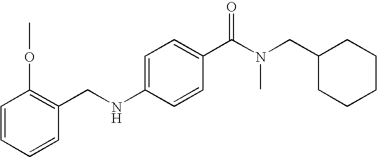 | [M + 1]⁺ 367 |
| 6-3 | 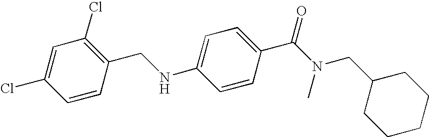 | [M + 1]⁺ 405 |
| 6-4 | 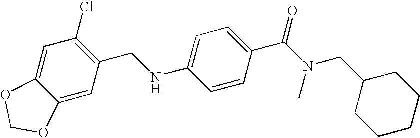 | [M + 1]⁺ 415 |
| 6-5 | 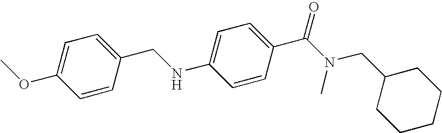 | [M + 1]⁺ 367 |
| 6-6 | 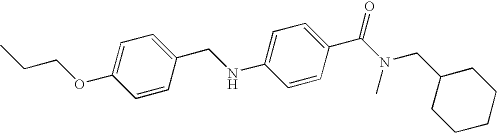 | [M + 1]⁺ 395 |
| 6-7 | 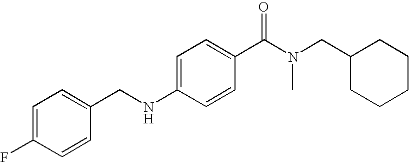 | [M + 1]⁺ 355 |
| 6-8 | 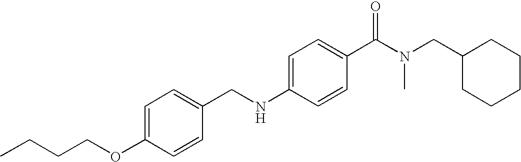 | [M + 1]⁺ 409 |
| 6-9 | 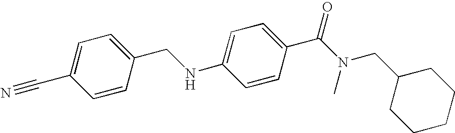 | [M + 1]⁺ 362 |
| 6-10 | 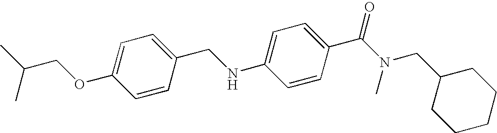 | [M + 1]⁺ 409 |
| 6-11 | 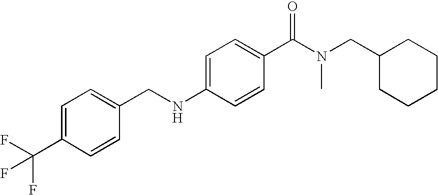 | [M + 1]⁺ 404 |
| 6-12 | 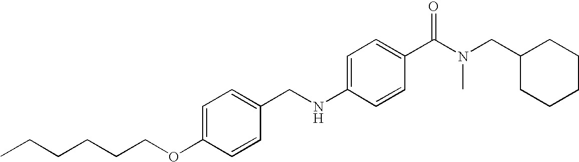 | [M + 1]⁺ 437 |
| 6-13 | 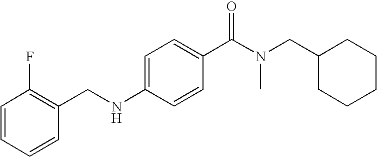 | [M + 1]⁺ 355 |
| 6-14 | 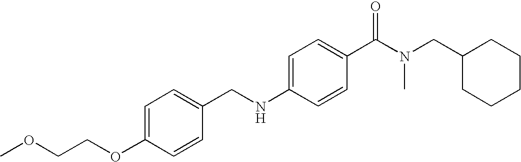 | [M + 1]⁺ 411 |

### EXAMPLE 7

The following compounds are prepared analogously to Examples 1 and 2.

| Compd | Structure | MS [m/z] |
|---|---|---|
| 7-1 | 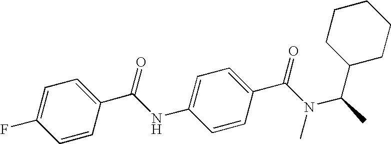 | [M + 1]⁺ 383 |
| 7-2 | 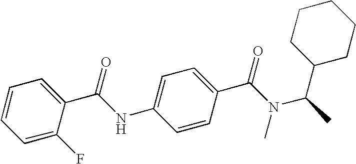 | [M + 1]⁺ 383 |
| 7-3 | 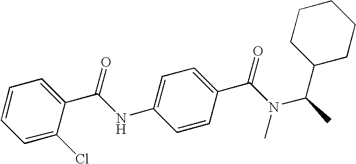 | [M + 1]⁺ 399 |
| 7-4 | 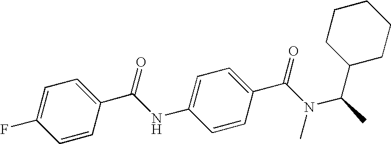 | [M + 1]⁺ 383 |
| 7-5 | 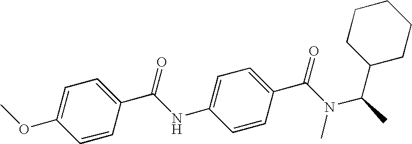 | [M + 1]⁺ 395 |
| 7-6 | 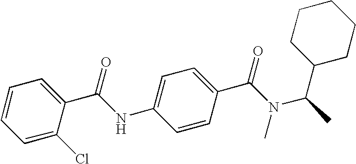 | [M + 1]⁺ 399 |
| 7-7 | 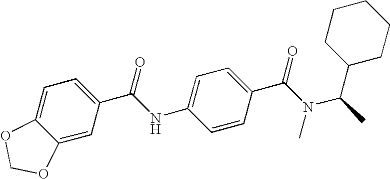 | [M + 1]⁺ 410 |
| 7-8 | 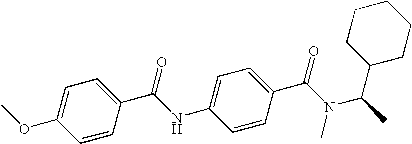 | [M + 1]⁺ 395 |
| 7-9 | 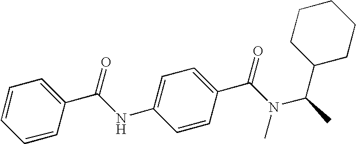 | [M + 1]⁺ 365 |
| 7-10 | 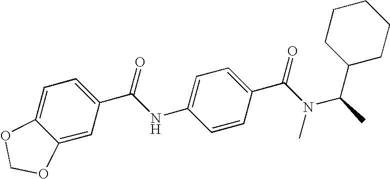 | [M + 1]⁺ 410 |
| 7-11 | 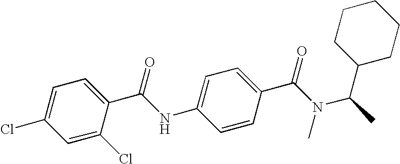 | [M + 1]⁺ 433 |
| 7-12 | 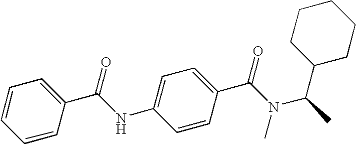 | [M + 1]⁺ 365 |
| 7-13 | 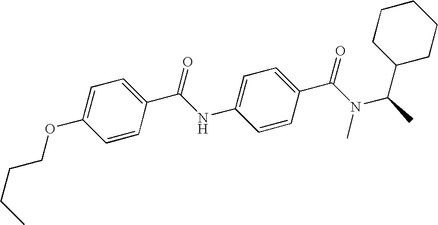 | [M + 1]⁺ 438 |
| 7-14 | 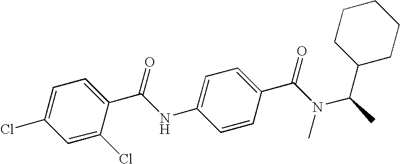 | [M + 1]⁺ 433 |
| 7-15 | 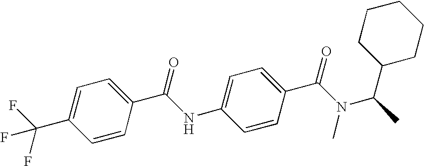 | [M + 1]⁺ 433 |
| 7-16 | 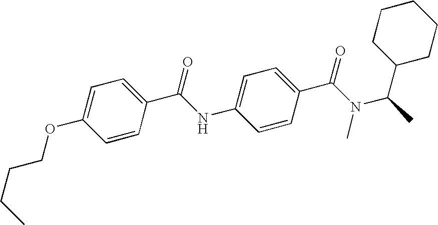 | [M + 1]⁺ 438 |
| 7-17 | 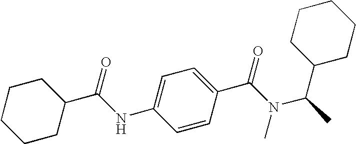 | [M + 1]⁺ 372 |
| 7-18 | 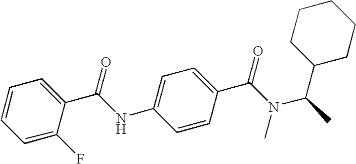 | [M + 1]⁺ 383 |
| 7-19 | 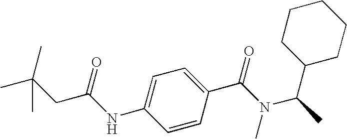 | [M + 1]⁺ 360 |
| 7-20 | 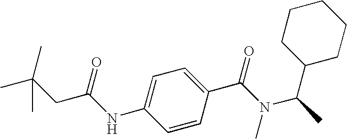 | [M + 1]⁺ 360 |
| 7-21 | 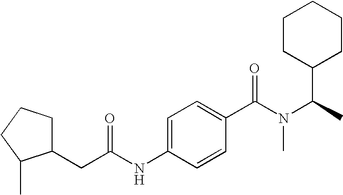 | [M + 1]⁺ 372 |
| 7-22 | 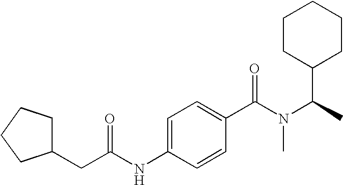 | [M + 1]⁺ 372 |
| 7-23 | 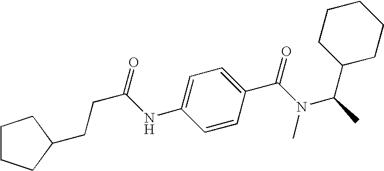 | [M + 1]⁺ 386 |
| 7-24 | 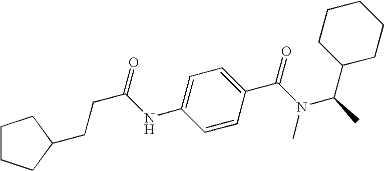 | [M + 1]⁺ 386 |
| 7-25 | 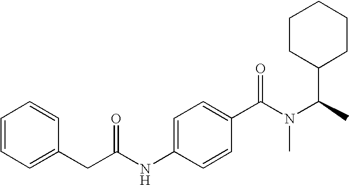 | [M + 1]⁺ 379 |
| 7-26 | 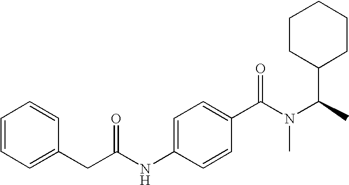 | [M + 1]⁺ 379 |
| 7-27 | 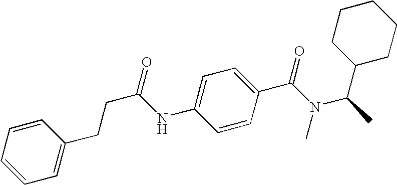 | [M + 1]⁺ 393 |
| 7-28 | 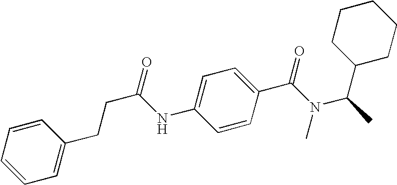 | [M + 1]⁺ 393 |

### EXAMPLE 8

The following compounds are prepared analogously to Example 1.

| Compd | Structure | MS [m/z] |
|---|---|---|
| 8-1 | 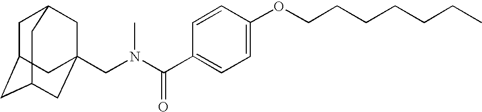 | [M + 1]⁺ 398 |
| 8-2 | 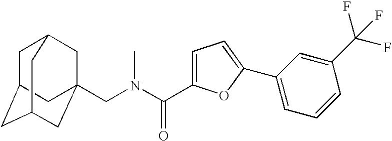 | [M + 1]⁺ 418 |
| 8-3 | 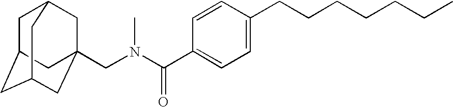 | [M + 1]⁺ 382 |
| 8-4 | 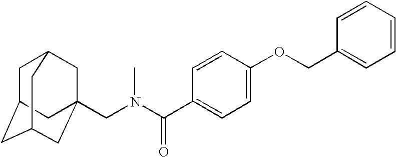 | [M + 1]⁺ 390 |
| 8-5 | 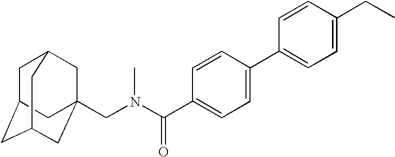 | [M + 1]⁺ 388 |
| 8-6 | 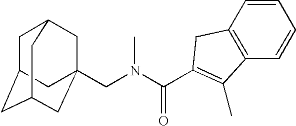 | [M + 1]⁺ 336 |
| 8-7 | 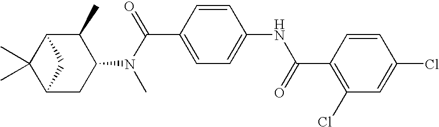 | (mp 107-114° C.) |
| 8-8 | 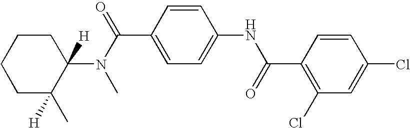 | (mp 80-90° C.) |
| 8-9 | 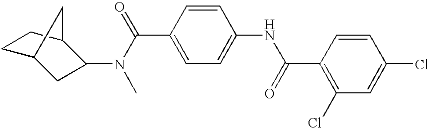 | (mp 85-100° C.) |
| 8-9 | 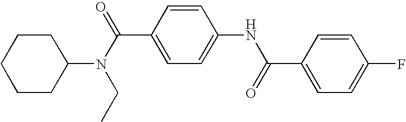 | |

### EXAMPLE 9

### 2,4-Dichloro-N-{4-[(4aS*,8aR*)-octahydro-1(2H)-quinoline-1-carbonyl]phenyl}-benzamide

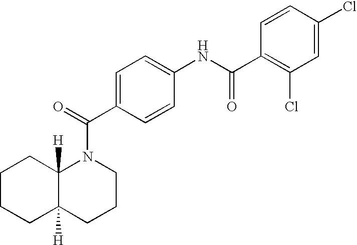

### A. (4-Nitrophenyl)-(4aS*,8aR*)-octahydro-1(2H)-quinolin-1-yl-methanone

To a solution of 10.0 g (71.8 mmol) of decahydroquinoline and 18.6 g (144 mmol, of diisopropylethylamine in 150 mL of dichloromethane cooled in an ice bath is added dropwise a solution of 13.33 g (71.8 mmol) of 4-nitrobenzoyl chloride. The mixture is stirred at RT for 18 h, then washed twice with 1 N aqueous HCl. The organic phase is dried over anhydrous Na₂SO₄ and the solvent is removed under reduced pressure. The residue is crystallized three times from diethyl ether/hexane then a final time from diethyl ether to give the trans product, (4-nitrophenyl)-(4aS*,8aR*)-octahydro-1(2H)-quinolin-1-yl-methanone: m.p. 84-87° C.; NMR (CDCl₃) 8.26 (d, 2H, J=7), 7.55 (d, 2H, J=7), 3.55-3.45 (m, 1H), 3.43-3.24 (m, 2H), 2.27 (m, 1H), 1.87-1.04 (m, 12H).

Alternatively, if the crude residue is chromatographed four times using hexane/EtOAc (60:40) as the eluent, the trans isomer can be separated from the cis isomer, (4-nitrophenyl)-(4aR*,8aR*)-octahydro-1(2H)-quinolin-1-yl-methanone: m.p. 103-106° C.; NMR (CDCl₃) 8.28 (m, 2H), 7.53 (d, 2H, J=7), 4.82-4.51 (m, 1H), 3.54-3.25 (m, 1H), 3.22-2.77 (m, 1H), 2.08-0.90 (m 13H).

### B. (4-Aminophenyl)-(4aS*,8aR*)-octahydro-1(2H)-quinolin-1-yl-methanone

A mixture of the title A compound, (4-nitrophenyl)-(4aS*,8aR*)-octahydro-1(2H)-quinolin-1-yl-methanone (2.0 g) and of 10% Pd/C (200 mg) in 100 mL ethanol (EtOH) is hydrogenated at 1 atm for 18 h. The catalyst is removed by vacuum filtration through Celite, and the filtrate is concentrated to give (4-aminophenyl)-(4aS*,8aR*)-octahydro-1(2H)-quinolin-1-yl-methanone. The product is used as such in the following step.

### C. 2,4-Dichloro-N-{4-[(4aS*,8aR*)-octahydro-1(2H)-quinoline-1-carbonyl]phenyl}-benzamide

To a solution of the title B compound, (4-aminophenyl)-(4aS*,8aR*)-octahydro-1(2H)-quinolin-1-yl-methanone (1.8 g, 6.9 mmol) and 1.8 g (13.8 mmol) of diisopropylethylamine in 10 mL of dichloromethane is added dropwise a solution of 1.4 g (6.9 mmol) of 2,4-dichlorobenzoyl chloride. After the mixture is stirred at RT for 24 h, it is poured into EtOAc. The mixture is washed twice with 1 N aqueous HCl, once with 8% aqueous sodium bicarbonate (NaHCO₃), and once with saturated sodium chloride. The organic phase is dried over sodium sulfate, the solvent is removed and the resulting solid is recrystallized from cold EtOH to give 2,4-dichloro-N-{4-[(4aR*,8aS*)-octahydro-1(2H)-quinoline-1-carbonyl]-phenyl]benzamide: m.p. 212-214° C.; NMR (DMSO-d₆) 10.69 (s, 1H), 7.79 (d, 1H, J=1.8), 7.73 (d, 2H, J=8.4), 7.65 (d, 1H, J=8.4), 7.57 (m, 1H), 7.36 (d, 2H, J=8.4), 3.34 (m, 3H), 2.10 (m, 1H), 1.77-0.98 (m, 12H).

### EXAMPLE 10

### 2,4-Dichloro-N-{4-[(4aR*,8aR*)-octahydro-1(2H)-quinoline-1-carbonyl]phenyl]benzamide

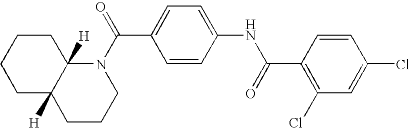

### A. (4-Aminophenyl)-(4aR*,8aR*)-octahydro-1(2H)-quinolin-1-yl-methanone

A solution of (4-nitrophenyl)-(4aR*,8aR*)-octahydro-1(2H)-quinolin-1-yl-methanone (200 mg, 0.69 mmol), prepared in step A of Example 9, in 75 mL of EtOH is hydrogenated at 1 atm over 10% Pd/C (20 mg) for 18 h. The catalyst is removed by vacuum filtration and the filtrate is concentrated under reduced pressure to give (4-aminophenyl)(4aR*,8aR*) octahydro-1(2H)-quinolin-1-yl-methanone.

### B. 4-Flouro-N-{4-[(4aR*,8aR*)-octahydro-1(2H)-quinoline-1-carbonyl]phenyl]-benzamide

To a solution of the title A compound, (4-aminophenyl)-(4aR*,8aR*)-octahydro-1(2H)-quinolin-1-yl-methanone (120 mg, 0.46 mmol) and 120 mg (0.92 mmol) of diisopropylethylamine in 25 mL of dichloromethane is added 74 mg (0.47 mmol) of 4-fluorobenzoyl chloride. The mixture is stirred at RT for 18 h then is washed with 1 N aqueous HCl and water. The organic phase is dried over sodium sulfate and the solvent is removed under reduced pressure to give an amorphous solid. This is recrystallized from EtOAc to give 4-flouro-N-{4-[(4aR*,8aR*)-octahydro-1(2H)-quinoline-1-carbonyl]-phenyl]benzamide: m.p. 134-136° C.; NMR (CDCl₃) 8.40 (m, 1H), 7.97 (m, 2H), 7.56 (m, 2H), 7.31 (m, 2H), 7.17 (t, 2H), 4.80-4.46 (m, 1H), 3.79-3.50 (m, 1H), 3.15-2.72 (m, 1H), 2.09-0.99 (m, 13H).

### EXAMPLE 11

The following compounds are prepared analogously to Examples 9 and 10 using either the title B compound in Example 9 or the title A compound in Example 10 and the appropriate N-derivatizing agent, such as an activated derivative of a carboxylic acid, a chloroformate, a sulfonyl chloride, an isocyanate or a thioisocyanate.

| Compd | Structure | MS [m/z] |
|---|---|---|
| 11-1 | 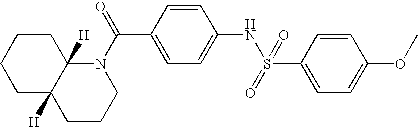 | [M + 1]⁺ 429 |
| 11-2 | 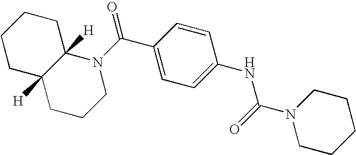 | [M + 1]⁺ 370 |
| 11-3 | 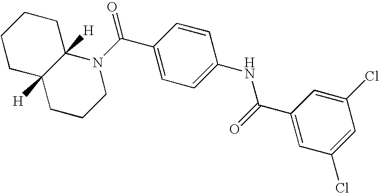 | [M + 1]⁺ 431 |
| 11-4 | 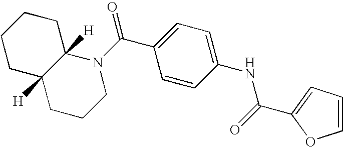 | [M + 1]⁺ 353 |
| 11-5 | 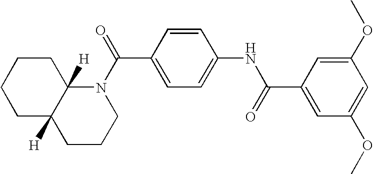 | [M + 1]⁺ 423 |
| 11-6 | 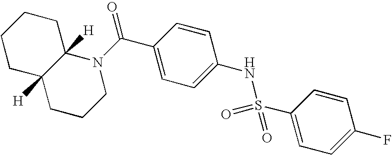 | [M + 1]⁺ 417 |
| 11-7 | 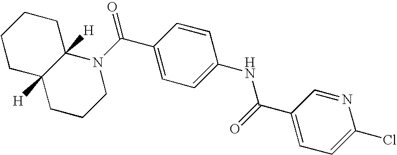 | [M + 1]⁺ 398 |
| 11-8 | 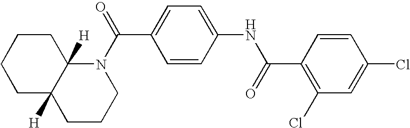 | [M + 1]⁺ 431 |
| 11-9 | 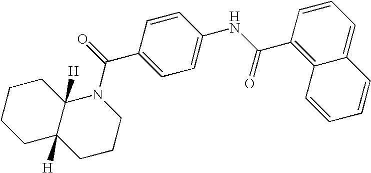 | [M + 1]⁺ 413 |
| 11-10 | 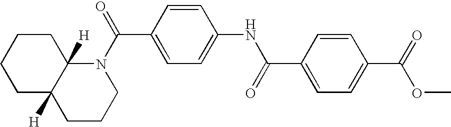 | [M + 1]⁺ 421 |
| 11-11 | 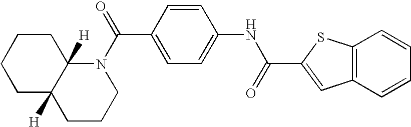 | [M + 1]⁺ 419 |
| 11-12 | 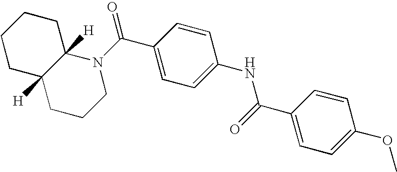 | [M + 1]⁺ 393 |
| 11-13 | 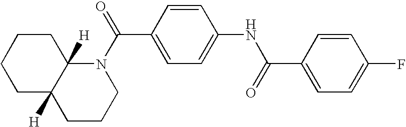 | [M + 1]⁺ 381 |
| 11-14 | 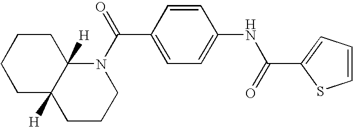 | [M + 1]⁺ 369 |
| 11-15 | 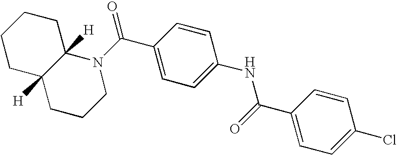 | [M + 1]⁺ 397 |
| 11-16 | 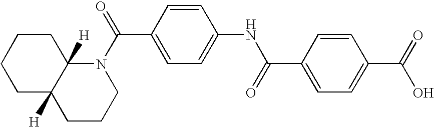 | [M + 1]⁺ 407 |
| 11-17 | 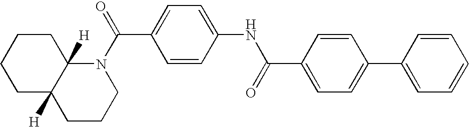 | [M + 1]⁺ 439 |
| 11-18 | 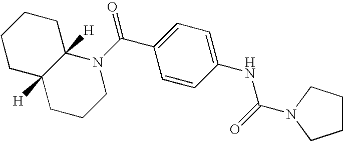 | [M + 1]⁺ 356 |
| 11-19 | 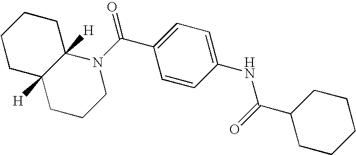 | [M + 1]⁺ 369 |
| 11-20 | 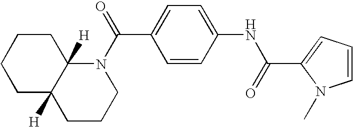 | [M + 1]⁺ 366 |
| 11-21 | 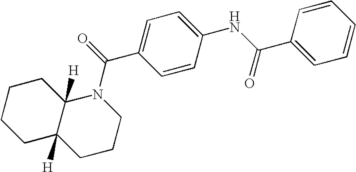 | [M + 1]⁺ 363 |
| 11-22 | 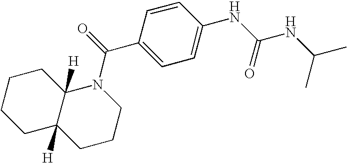 | [M + 1]⁺ 344 |
| 11-23 | 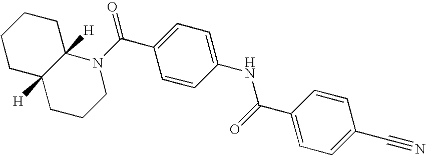 | [M + 1]⁺ 388 |
| 11-24 | 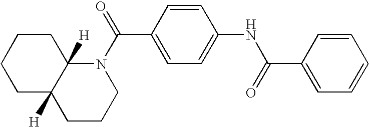 | [M + 1]⁺ 392 |
| 11-25 | 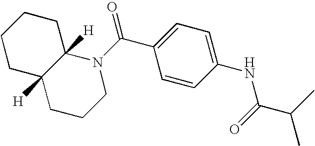 | [M + 1]⁺ 329 |
| 11-26 | 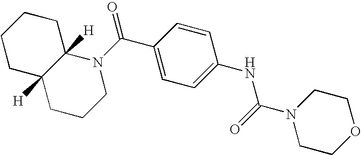 | [M + 1]⁺ 372 |
| 11-27 | 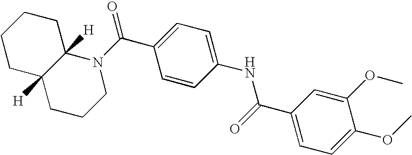 | [M + 1]⁺ 423 |
| 11-28 | 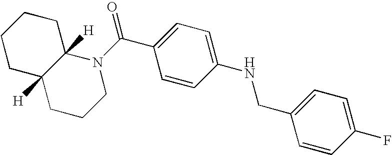 | [M + 1]⁺ 367 |
| 11-29 | 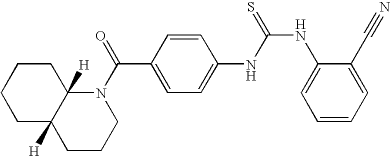 | [M + 1]⁺ 419 |
| 11-30 | 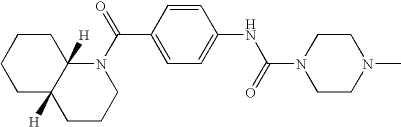 | [M + 1]⁺ 385 |
| 11-31 | 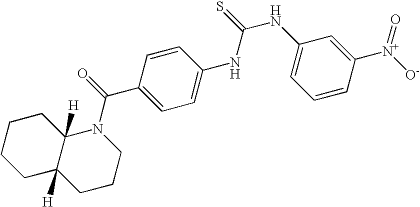 | [M + 1]⁺ 439 |
| 11-32 | 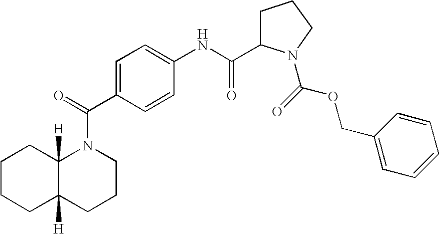 | [M + 1]⁺ 490 |
| 11-33 | 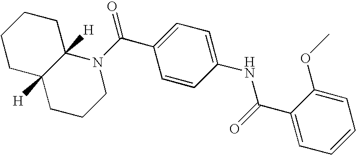 | [M + 1]⁺ 393 |
| 11-34 | 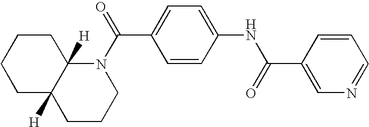 | [M + 1]⁺ 364 |
| 11-35 | 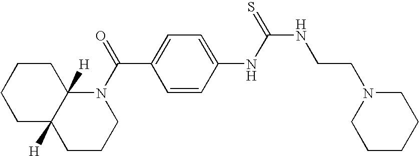 | [M + 1]⁺ 429 |
| 11-36 | 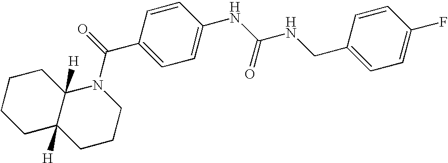 | [M + 1]⁺ 410 |
| 11-37 | 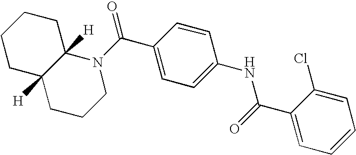 | [M + 1]⁺ 397 |
| 11-38 | 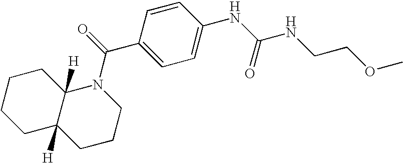 | [M + 1]⁺ 360 |
| 11-39 | 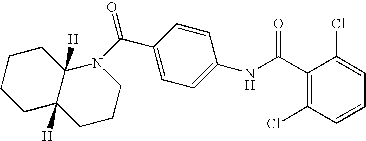 | [M + 1]⁺ 431 |
| 11-40 | 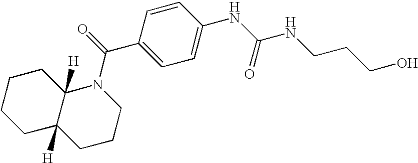 | [M + 1]⁺ 360 |
| 11-41 | 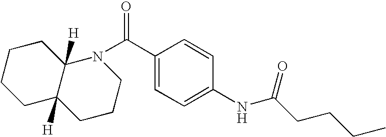 | [M + 1]⁺ 343 |
| 11-42 | 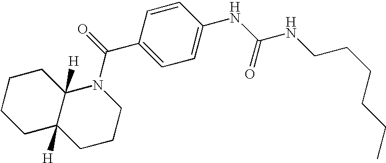 | [M + 1]⁺ 386 |
| 11-43 | 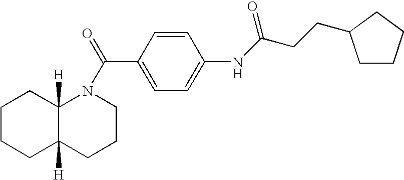 | [M + 1]⁺ 383 |
| 11-44 | 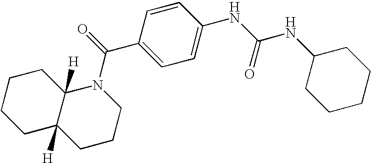 | [M + 1]⁺ 384 |
| 11-45 | 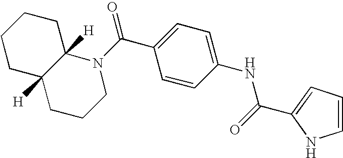 | [M + 1]⁺ 352 |
| 11-46 | 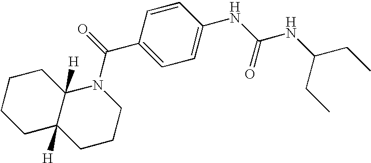 | [M + 1]⁺ 372 |
| 11-47 | 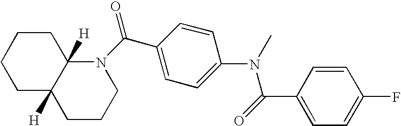 | [M + 1]⁺ 395 |
| 11-48 | 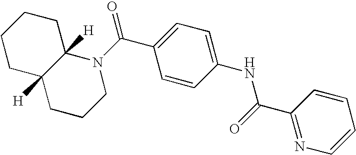 | [M + 1]⁺ 364 |
| 11-49 | 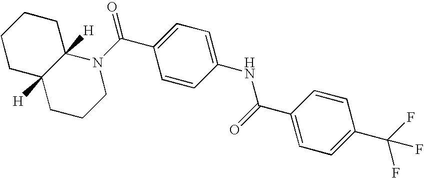 | [M + 1]⁺ 431 |
| 11-50 | 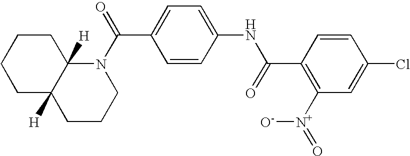 | [M + 1]⁺ 442 |
| 11-51 | 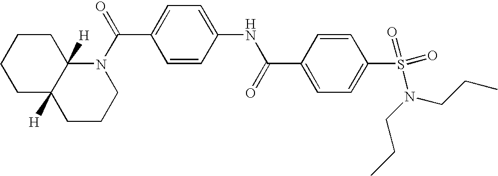 | [M + 1]⁺ 526 |
| 11-52 | 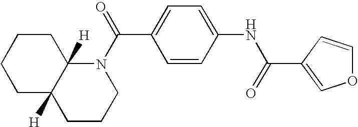 | [M + 1]⁺ 353 |
| 11-53 | 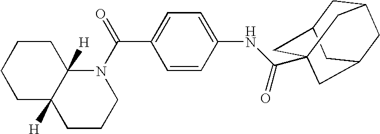 | [M + 1]⁺ 421 |
| 11-54 | 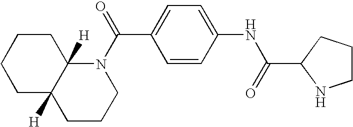 | [M + 1]⁺ 356 |
| 11-55 | 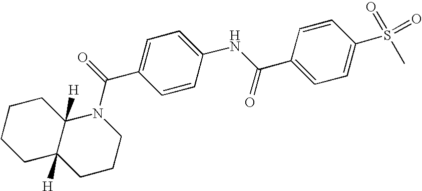 | [M + 1]⁺ 441 |
| 11-56 | 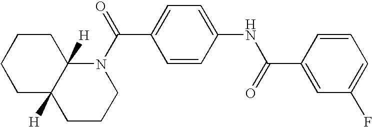 | [M + 1]⁺ 381 |
| 11-57 | 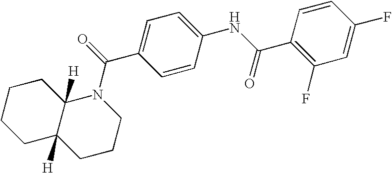 | [M + 1]⁺ 399 |
| 11-58 | 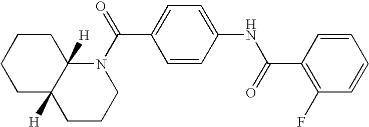 | [M + 1]⁺ 381 |
| 11-59 | 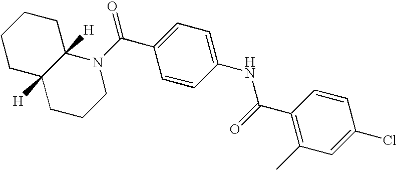 | [M + 1]⁺ 411 |
| 11-60 | 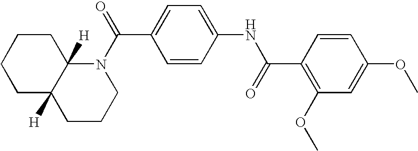 | [M + 1]⁺ 423 |
| 11-61 | 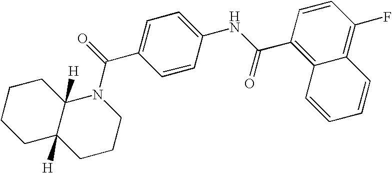 | [M + 1]⁺ 431 |
| 11-62 | 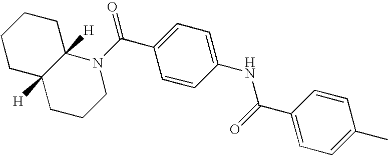 | [M + 1]⁺ 377 |
| 11-63 | 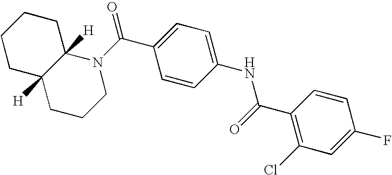 | [M + 1]⁺ 415 |
| 11-64 | 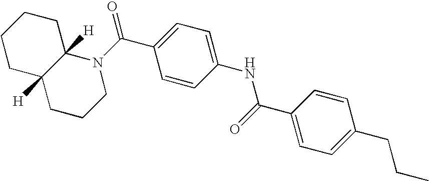 | [M + 1]⁺ 405 |
| 11-65 | 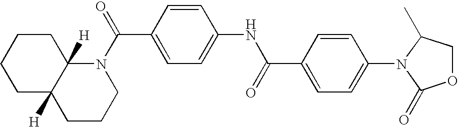 | [M + 1]⁺ 462 |
| 11-66 | 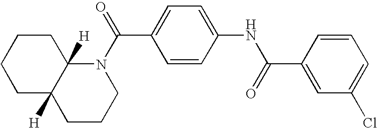 | [M + 1]⁺ 397 |
| 11-67 | 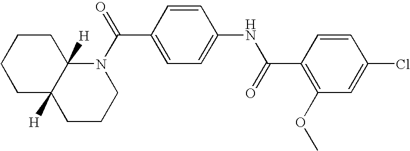 | [M + 1]⁺ 427 |
| 11-68 | 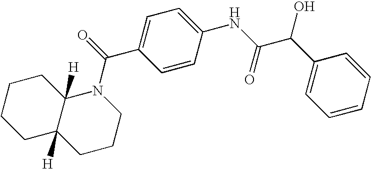 | [M + 1]⁺ 393 |
| 11-69 | 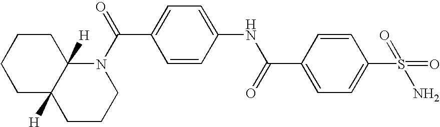 | [M + 1]⁺ 442 |
| 11-70 | 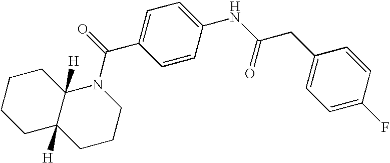 | [M + 1]⁺ 395 |
| 11-71 | 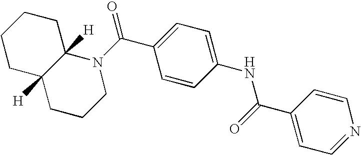 | [M + 1]⁺ 364 |
| 11-72 | 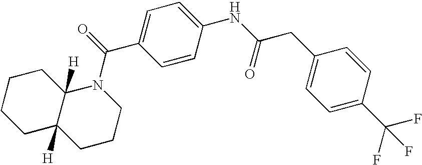 | [M + 1]⁺ 445 |
| 11-73 | 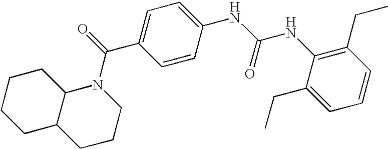 | [M + 1]⁺ 434 |
| 11-74 | 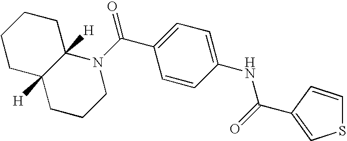 | [M + 1]⁺ 369 |
| 11-75 | 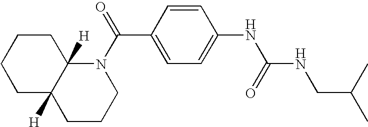 | [M + 1]⁺ 358 |
| 11-76 | 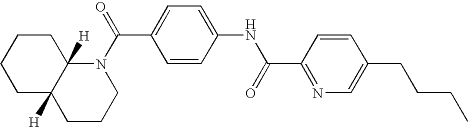 | [M + 1]⁺ 420 |

### EXAMPLE 12

The following compounds are prepared analogously to Example 9 starting from 2-chloro-4-nitrobenzoyl chloride and decahydroquinoline and treating the intermediate 4-amino-2-chlorobenzamide derivative analogous to the title B compound in Example 9 with the appropriate N-derivatizing agent, such as an activated derivative of a carboxylic acid, a chloroformate or an isocyanate.

| Compd | Structure | MS [m/z] |
|---|---|---|
| 12-1 | 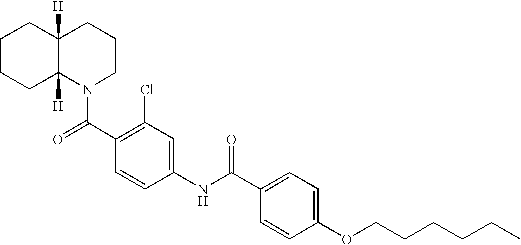 | [M + 1]⁺ 497 |
| 12-2 | 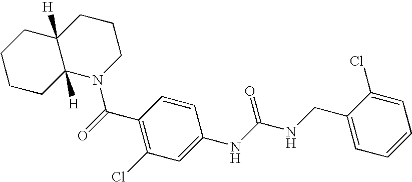 | [M + 1]⁺ 461 |
| 12-3 | 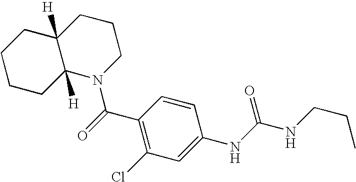 | [M + 1]⁺ 378 |
| 12-4 | 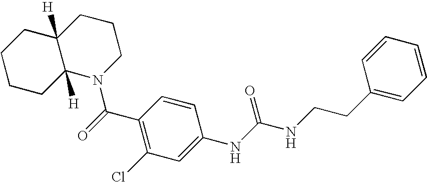 | [M + 1]⁺ 441 |
| 12-5 | 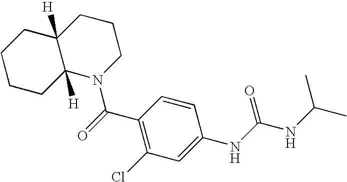 | [M + 1]⁺ 378 |
| 12-6 | 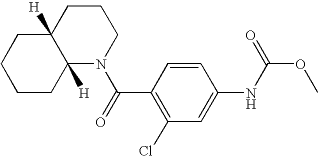 | [M + 1]⁺ 351 |
| 12-7 | 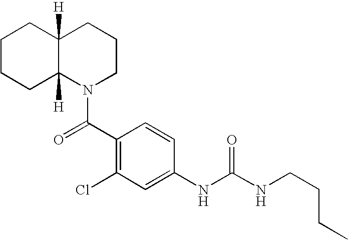 | [M + 1]⁺ 392 |
| 12-8 | 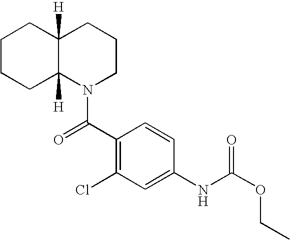 | [M + 1]⁺ 365 |
| 12-9 | 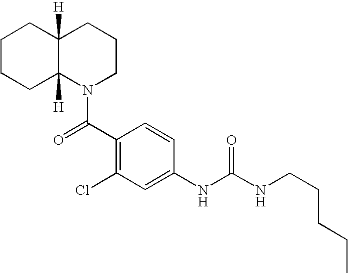 | [M + 1]⁺ 406 |
| 12-10 | 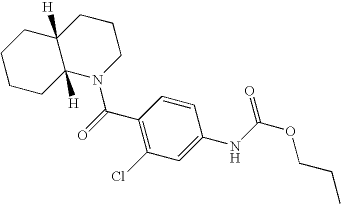 | [M + 1]⁺ 379 |
| 12-11 | 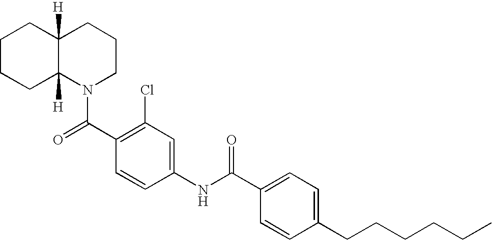 | [M + 1]⁺ 482 |
| 12-12 | 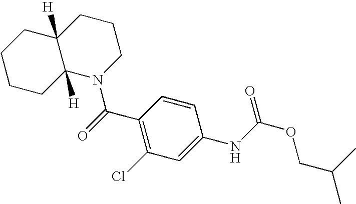 | [M + 1]⁺ 393 |
| 12-13 | 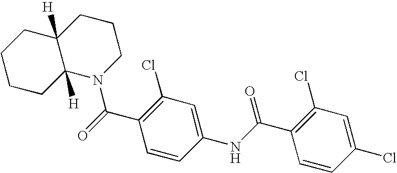 | [M + 1]⁺ 465 |
| 12-14 | 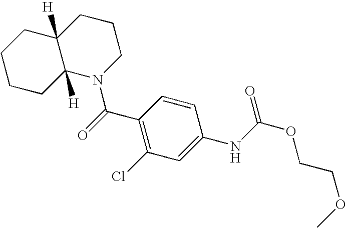 | [M + 1]⁺ 395 |
| 12-15 | 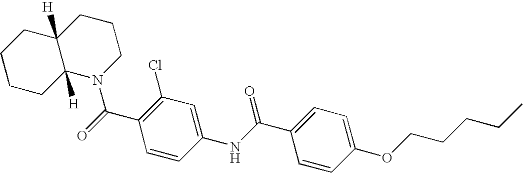 | [M + 1]⁺ 484 |
| 12-16 | 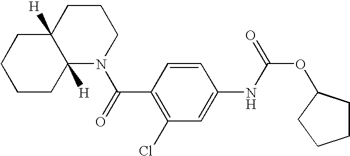 | [M + 1]⁺ 405 |
| 12-17 | 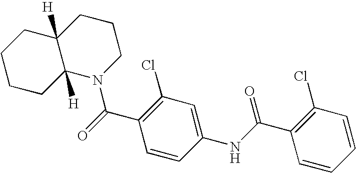 | [M + 1]⁺ 431 |
| 12-18 | 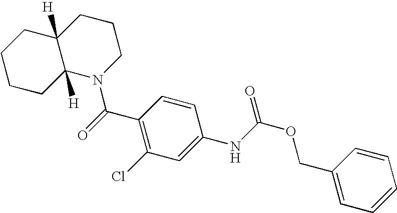 | [M + 1]⁺ 428 |
| 12-19 | 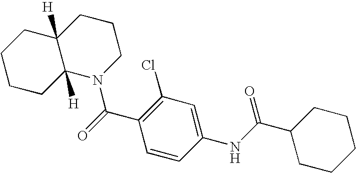 | [M + 1]⁺ 403 |
| 12-20 | 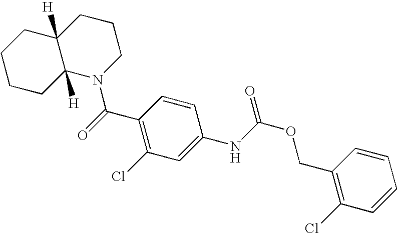 | [M + 1]⁺ 462 |
| 12-21 | 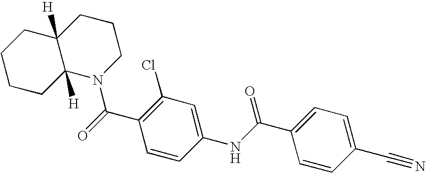 | [M + 1]⁺ 422 |
| 12-22 | 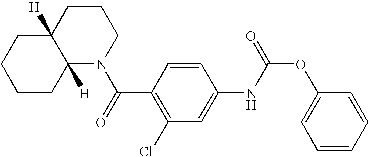 | [M + 1]⁺ 414 |
| 12-23 | 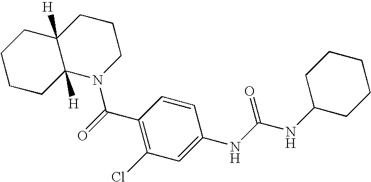 | [M + 1]⁺ 418 |
| 12-24 | 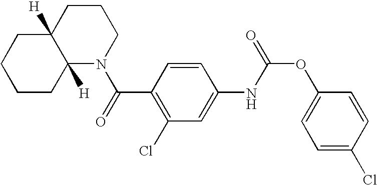 | [M + 1]⁺ 447 |
| 12-25 | 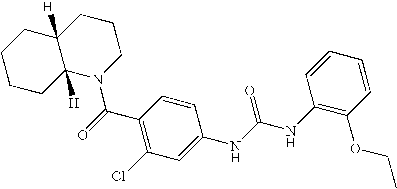 | [M + 1]⁺ 456 |
| 12-26 | 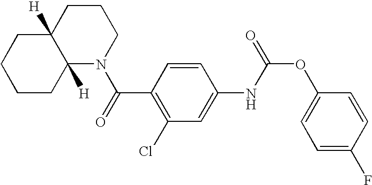 | [M + 1]⁺ 432 |
| 12-27 | 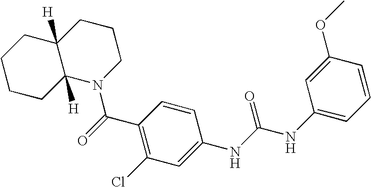 | [M + 1]⁺ 442 |
| 12-28 | 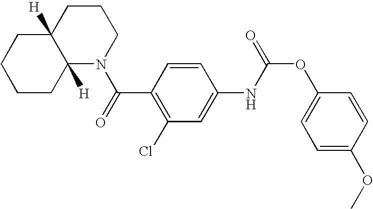 | [M + 1]⁺ 444 |
| 12-29 | 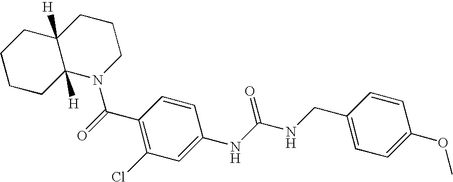 | [M + 1]⁺ 456 |
| 12-30 | 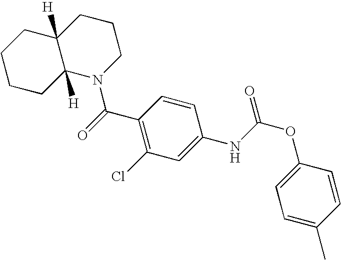 | [M + 1]⁺ 428 |
| 12-31 | 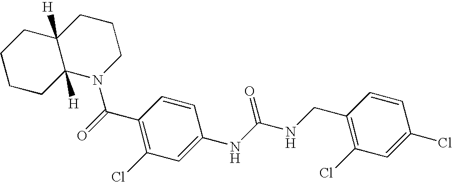 | [M + 1]⁺ 496 |
| 12-32 | 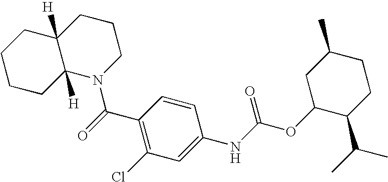 | [M + 1]⁺ 476 |
| 12-33 | 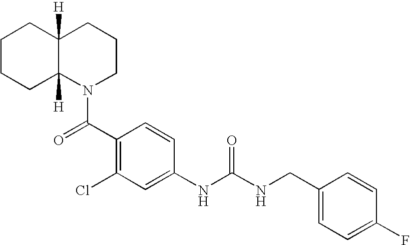 | [M + 1]⁺ 444 |
| 12-34 | 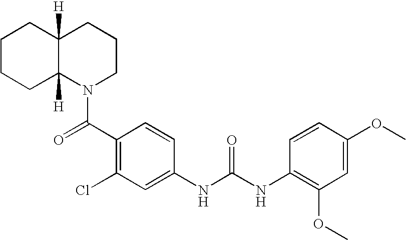 | [M + 1]⁺ 473 |
| 12-35 | 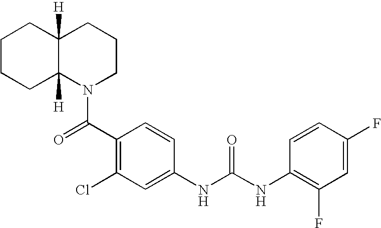 | [M + 1]⁺ 448 |
| 12-36 | 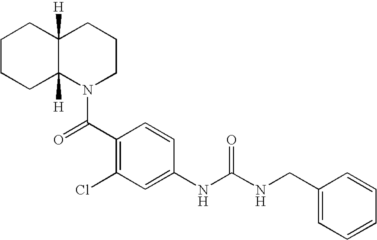 | [M + 1]⁺ 427 |

### EXAMPLE 13

The following compounds are prepared analogously to Example 9 starting from 2-methoxy-4-nitrobenzoyl chloride or 3-methoxy-4-nitrobenzoyl chloride and decahydro-quinoline, and treating the intermediate 4-amino-2-methoxybenzamide or 4-amino-3-methoxybenzamide derivatives analogous to the title B compound in Example 9 or the title A compound in Example 10 with the appropriate N-derivatizing agent, such as an activated derivative of a carboxylic acid, a chloroformate or an isocyanate.

| Compd | Structure | MS [m/z] |
|---|---|---|
| 13-1 | 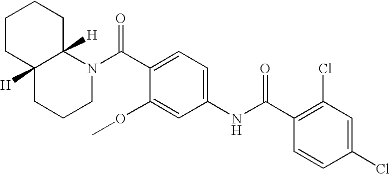 | [M + 1]⁺ 461 |
| 13-2 | 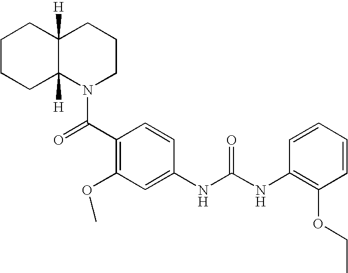 | [M + 1]⁺ 452 |
| 13-3 | 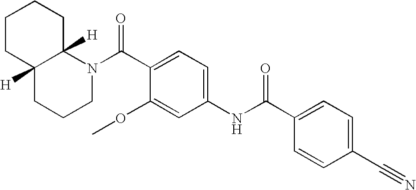 | [M + 1]⁺ 418 |
| 13-4 | 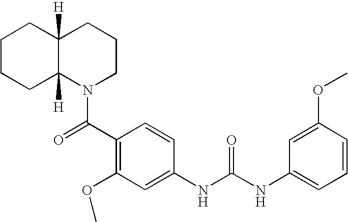 | [M + 1]⁺ 438 |
| 13-5 | 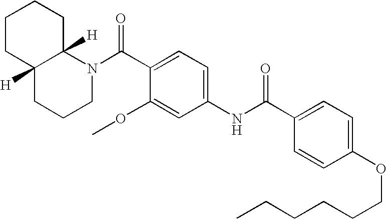 | [M + 1]⁺ 493 |
| 13-6 | 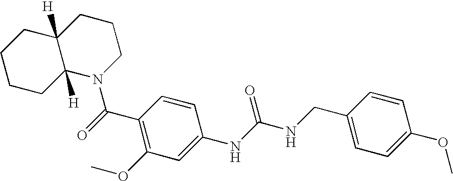 | [M + 1]⁺ 452 |
| 13-7 | 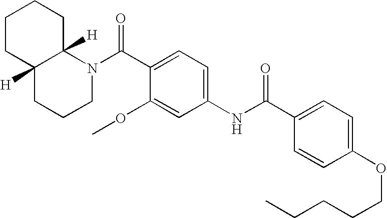 | [M + 1]⁺ 479 |
| 13-8 | 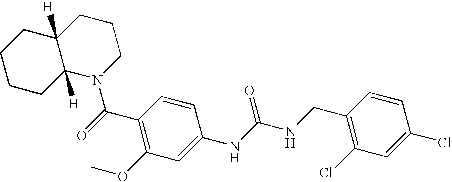 | [M + 1]⁺ 490 |
| 13-9 | 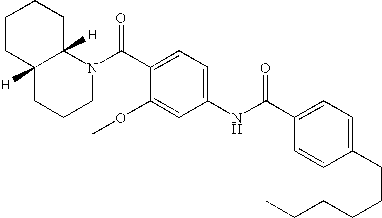 | [M + 1]⁺ 477 |
| 13-10 | 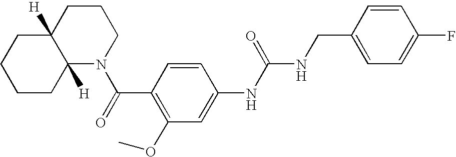 | [M + 1]⁺ 440 |
| 13-11 | 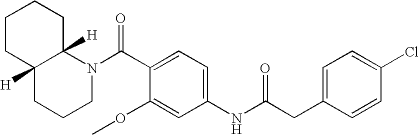 | [M + 1]⁺ 441 |
| 13-12 | 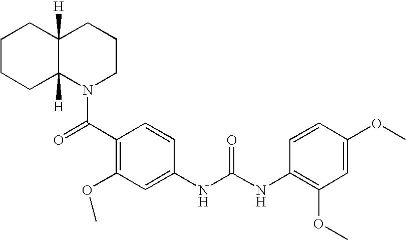 | [M + 1]⁺ 469 |
| 13-13 | 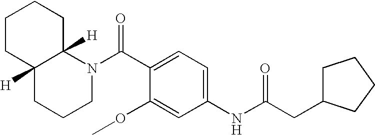 | [M + 1]⁺ 399 |
| 13-14 | 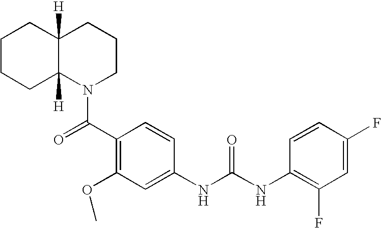 | [M + 1]⁺ 445 |
| 13-15 | 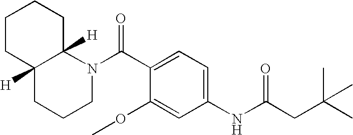 | [M + 1]⁺ 387 |
| 13-16 | 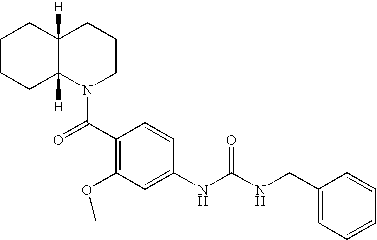 | [M + 1]⁺ 423 |
| 13-17 | 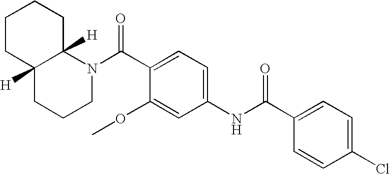 | [M + 1]⁺ 427 |
| 13-18 | 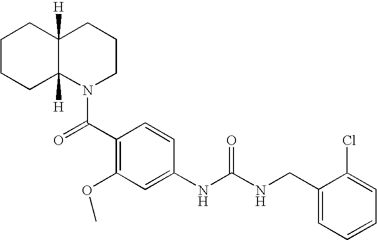 | [M + 1]⁺ 457 |
| 13-19 | 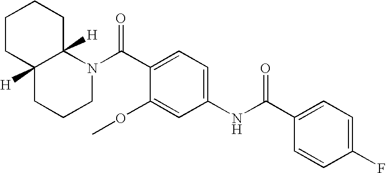 | [M + 1]⁺ 411 |
| 13-20 | 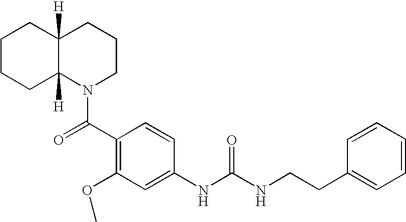 | [M + 1]⁺ 437 |
| 13-21 | 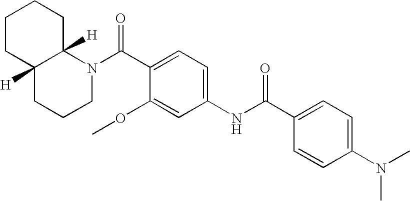 | [M + 1]⁺ 436 |
| 13-22 | 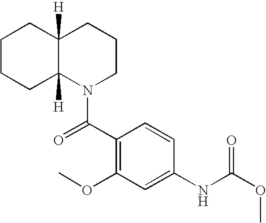 | [M + 1]⁺ 347 |
| 13-23 | 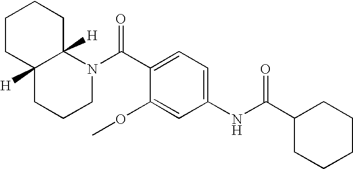 | [M + 1]⁺ 399 |
| 13-24 | 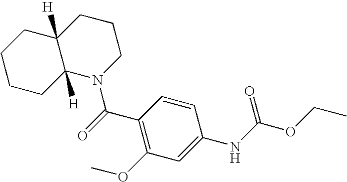 | [M + 1]⁺ 361 |
| 13-25 | 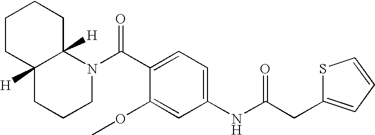 | [M + 1]⁺ 413 |
| 13-26 | 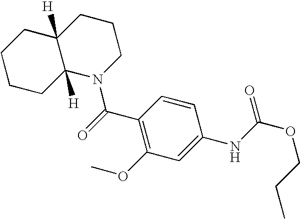 | [M + 1]⁺ 375 |
| 13-27 | 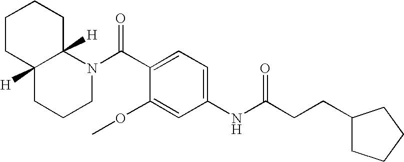 | [M + 1]⁺ 413 |
| 13-28 | 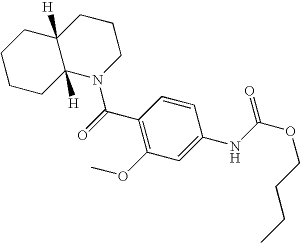 | [M + 1]⁺ 390 |
| 13-29 | 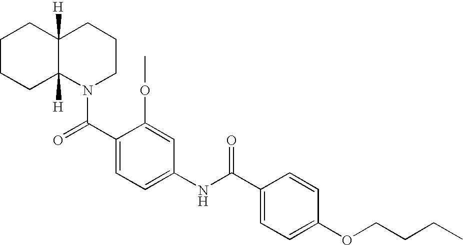 | [M + 1]⁺ 466 |
| 13-30 | 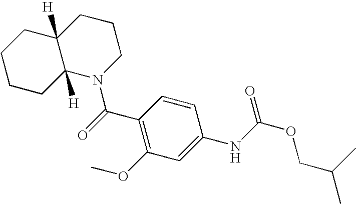 | [M + 1]⁺ 390 |
| 13-31 | 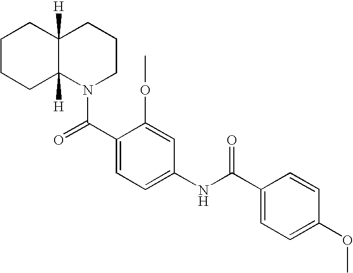 | [M + 1]⁺ 423 |
| 13-32 | 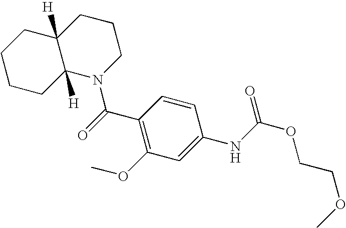 | [M + 1]⁺ 392 |
| 13-33 | 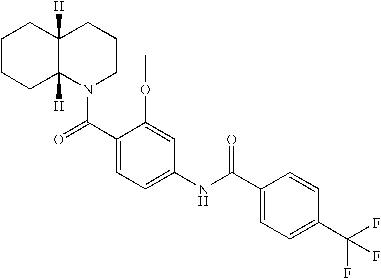 | [M + 1]⁺ 461 |
| 13-34 | 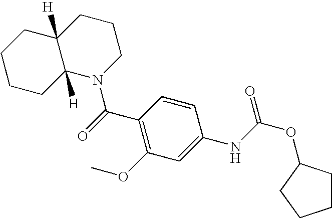 | [M + 1]⁺ 402 |
| 13-35 | 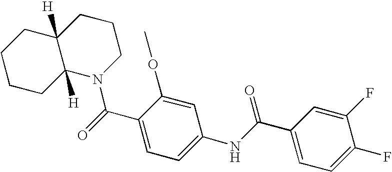 | [M + 1]⁺ 429 |
| 13-36 | 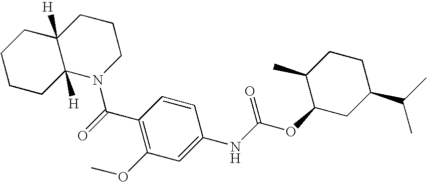 | [M + 1]⁺ 472 |
| 13-37 | 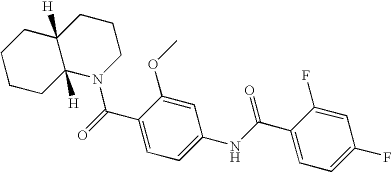 | [M + 1]⁺ 429 |
| 13-38 | 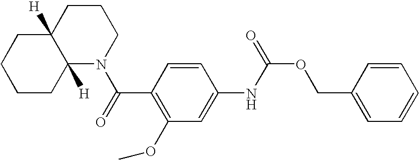 | [M + 1]⁺ 424 |
| 13-39 | 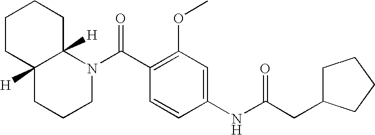 | [M + 1]⁺ 400 |
| 13-40 | 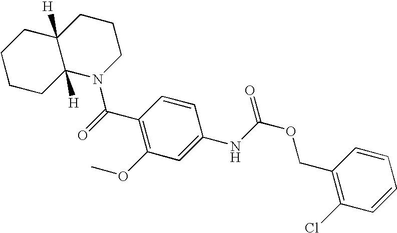 | [M + 1]⁺ 458 |
| 13-41 | 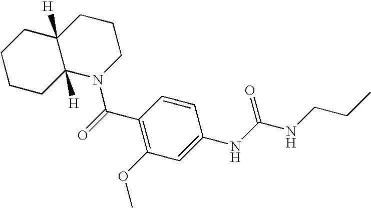 | [M + 1]⁺ 374 |
| 13-42 | 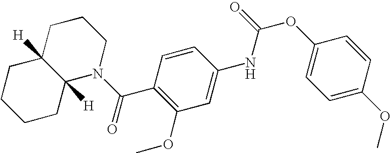 | [M + 1]⁺ 440 |
| 13-43 | 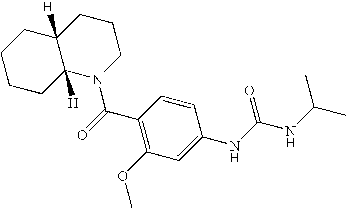 | [M + 1]⁺ 374 |
| 13-44 | 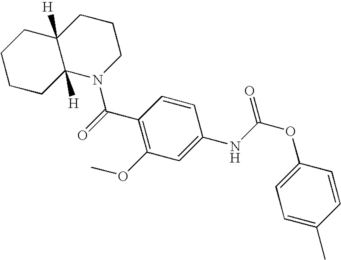 | [M + 1]⁺ 424 |
| 13-45 | 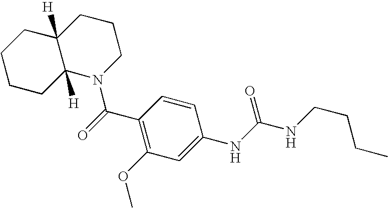 | [M + 1]⁺ 388 |
| 13-46 | 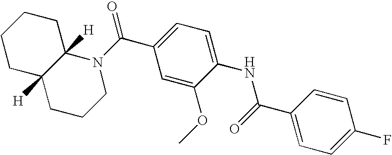 | [M + 1]⁺ 411 |
| 13-47 | 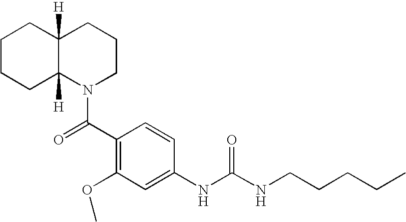 | [M + 1]⁺ 402 |
| 13-48 | 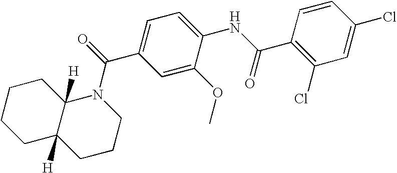 | [M + 1]⁺ 461 |
| 13-49 | 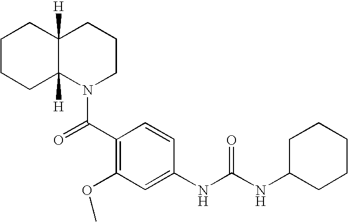 | [M + 1]⁺ 414 |
| 13-50 | 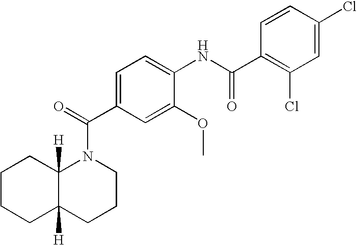 | [M + 1]⁺ 461 |

### EXAMPLE 14

### {3-Chloro-4[(4aS*,8aR*)-octahydro-1(2H)-quinoline-1-carbonyl]phenyl}methylcarbamic acid 4-methoxyphenyl ester

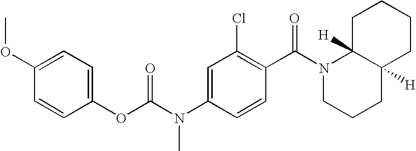

### A. {3-Chloro-4-[(4aS*,8aR*)-octahydro-1(2H)-quinoline-1-carbonyl]phenyl}methylcarbamic acid allyl ester

A solution of the (4-amino-2-chlorophenyl)-octahydro-1(2H)-quinolin-1-yl-methanone, prepared as illustrated in Example 12, (730 mg, 2.50 mmol) in 20 mL THF is treated sequentially with NMM (0.41 mL, 3.75 mmol) and allyl chlorformate (0.37 mL, 3.25 mmol). The reaction is stirred at RT for 16 h, then partioned between EtOAc and water. The organic layer is washed with brine, dried over anhydrous Na₂SO₄, and concentrated to afford a yellow foam. The residue is taken up in 20 mL DMF and treated with sodium hydride (150 mg, 3.75 mmol). After stirring at RT for 10 min, iodomethane (0.20 mL, 3.25 mmol) is added. The reaction is stirred at RT for 4 h further, then quenched with saturated aqueous ammonium chloride. The product is taken up in EtOAc and the organic layer is washed sequentially with saturated aqueous lithium chloride and brine, dried over anhydrous Na₂SO₄, and concentrated to afford {3-chloro-4-[(4aS*,8aR*)-octahydro-1(2H)-quinolin-1-carbonyl]-phenyl}methylcarbamic acid allyl ester: API-MS 391 [M+H]⁺. The product is used without purification.

### B. (2-Chloro-4-methylamino-phenyl)-(4aS*,8aR*)-octahydro-1(2H)-quinolin-1-yl-methanone

A solution of the title A compound, {3-chloro-4-[(4aS*,8aR*)-octahydro-1(2H)-quinolin-1-carbonyl]-phenyl}methylcarbamic acid allyl ester (975 mg, 2.50 mmol) in 22 mL acetonitrile/water (10:1) is treated sequentially with morpholine (0.65 mL, 7.5 mmol), 3,3′,3″-phospinidyne-tris(benzenesulfonic acid) trisodium salt (284 mg, 7.5 mmol) and palladium(II) acetate (561 mg, 2.5 mmol). The mixture is stirred at RT for 3 h, then partitioned between EtOAc and water. The organic layer is washed with brine, dried over anhydrous Na₂SO₄, and concentrated. Purification by chromatography (eluent 30% EtOAc in hexanes) affords (2-chloro-4-methylamino-phenyl)-(4aS*,8aR*)-octahydro-1(2H)-quinolin-1-yl-methanone as a yellow oil: NMR (DMSO-d₆) 1.00-1.44 (m), 1.55-1.76 (m), 2.33 (br s, 1H), 2.67 (d, 3H, J=5.1), 2.92 (d, 1H, J=6.4), 3.26 (br s), 3.56 (t, 2H, J=4.5), 5.15-5.21 (m, 1H), 5.73-5.86 (m, 1H), 6.16 (app q, 1H, J=4.7), 6.48-6.51 (m, 2H), 6.89 (br s, 1H); API-MS 307 [M+H]⁺.

### C. {3-Chloro-4-[(4aS*,8aR*)-octahydro-quinoline-1-carbonyl]-phenyl}methylcarbamic acid 4-methoxyphenyl ester

Under multiparallel solution phase synthesis conditions, a solutions of NMM (2.0 M in THF, 150 μL, 0.30 mmol) and p-methoxyphenyl chloroformate (1.0 M in THF, 225 μL, 0.225 mmol) are dispensed sequentially into a vial containing a solution of the title B compound, (2-chloro-4-methylamino-phenyl)-(4aS*,8aR*)-octahydro-1(2H)-quinolin-1-yl-methanone (0.43 M in DMF, 349 μL, 0.15 mmol). The vial is shaken at RT for 16 h, then an aqueous solution of lithium hydorxide (1.5 N, 150 μL, 0.225 mmol) is dispensed into the vial, and the vial is shaken for additional 15 min. The reaction mixture is acidified with 50 μL TFA, and purification on HPLC affords 3-chloro-4-[(4aS*,8aR*)-octahydro-1(2H)-quinoline-1-carbonyl]-phenyl}-methylcarbamic acid 4-methoxyphenyl ester: API-MS 458 [M+H]⁺.

### EXAMPLE 15

### 1-{3-Chloro-4-[(4aS*,8aR*)-octahydro-1(2H)-quinoline-1-carbonyl]phenyl}-3-(3-methoxyphenyl)-1-methylurea

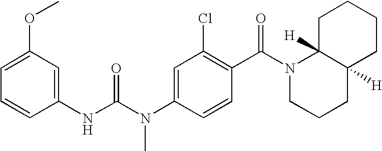

The title compound is prepared analogously to Example 14: API-MS 457 [M+H]⁺.

### EXAMPLE 16

### 1-{3-Chloro-4-[(4aS*,8aR*)-octahydro-1(2H)-quinoline-1-carbonyl]-phenyl}-3-(2,4-dichloro-benzyl)-1-methylurea

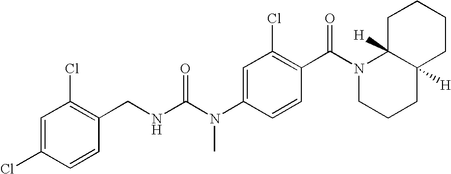

The title compound is prepared analogously to Example 14: API-MS 510 [M+H]⁺.

### EXAMPLE 17

### 2,4-Dichloro-N-[4-((4aS*,8aR*)-octahydro-1(2H)-quinoline-1-carbonyl)-3-propoxy-phenyl]benzamide

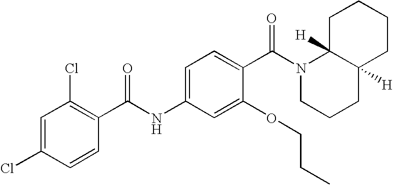

### A. 2-Hydroxy-4-nitrobenzoic acid

A mixture of 2-methoxy-4-nitrobenzoic acid (5.00 g, 25.38 mol), 25 mL 48% HBr, and 25 mL glacial acetic acid is heated at 90° C. for 72 h. The mixture is cooled to RT and poured into ice-water. The product is collected by vacuum filtration, washed with water, and dried in a vacuum oven, at 50° C. for 16 h to obtain 2-hydroxy-4-nitrobenzoic acid as a pale yellow solid: NMR (DMSO-d₆) 7.69-7.73 (m, 2H), 7.99-8.02 (m, 1H), 12.55 (br s, 1H); API-MS 182: [M−H]⁻.

### B. 2-Allyloxy-4-nitrobenzoic acid allyl ester

A solution of the title A compound, 2-hydroxy-4-nitrobenzoic acid (1.937 g, 10.58 mmol) in 40 mL of DMF is treated with sodium hydride (931 mg, 23.28 mmol). After stirring at RT for 20 min, allyl bromide (2.61 mL, 23.28 mmol) is added, and the reaction is strirred at RT for 16 h. The reaction is quenched with 1 N aqueous HCl, and the product is taken up in EtOAc. The organic layer is washed sequentially with saturated aqueous lithium chloride and brine, dried over anhydrous Na₂SO₄, and concentrated. Purification by flash chromatography (10% EtOAc in hexane) affords 2-allyloxy-4-nitrobenzoic acid allyl ester as a yellow oil: NMR (CDCl₃) 4.72-4.74 (m, 2H), 4.83-4.86 (m, 2H), 5.29-5.57 (m, 4H), 5.97-6.13 (m, 2H), 7.80-7.94 (m, 3H).

### C. 2-Allyloxy-4-nitrobenzoic acid

A solution of sodium hydride (1.13 g, 28.23 mmol) dissolved in 10 mL of water is added to a solution of the title B compound, 2-allyloxy-4-nitrobenzoic acid allyl ester (1.49 g, 5.65 mmol) in 40 mL of THF. The reaction is stirred at RT for 16 h, then acidified with 1 N aqueous HCl. The product is taken up in EtOAc, and the organic layer is washed with brine, dried over anhydrous Na₂SO₄, and concentrated to afford 2-allyloxy-4-nitrobenzoic acid as a pale yellow solid: NMR (CDCl₃) 4.89 (d, 2H, J=5.3), 5.48-5.61 (m, 2H), 6.05-6.18 (m, 1H), 7.84-7.98 (m, 2H), 8.29-8.33 (m, 1H).

### D. (2-Allyloxy-4-nitrophenyl)-[octahydro-1(2H)-quinolin-1-yl]-methanone

Oxalyl chloride (0.65 mL, 7.47 mmol) is added dropwise to a solution of the title C compound, 2-allyloxy-4-nitrobenzoic acid (1.11 g, 4.98 mmol) in 0.50 mL DMF and 40 mL CH₂Cl₂ at 0° C. The reaction is stirred at 0° C. for 1 h then NMM (1.37 mL, 12.45 mmol) and decahydroquinoline (832 mg, 5.97 mmol) are added sequentially. The reaction is warmed to RT and stirred for 3 h. The mixture is partitioned between EtOAc and 1 N aqueous NaOH. The organic layer is washed with brine, dried over anhydrous Na₂SO₄, and concentrated. Purification by flash chromatography (25% EtOAc in hexane) affords (2-allyloxy-4-nitrophenyl)-[octahydro-1(2H)-quinolin-1-yl]-methanone as a yellow oil: NMR (CDCl₃) 1.24-1.76 (m, 13H), 2.42 (br s, 1H), 3.01-3.58 (m, 2H), 4.64 (br s, 2H), 5.38 (app dd, 2H, J=30.1, 9.8), 5.96-5.99 (m, 1H), 7.33-7.42 (m, 1H), 7.73-7.89 (m, 2H); API-MS 345 [M+H]⁺.

### E. (4-Amino-2-propoxyphenyl)-[octahydro-1(2H)-quinolin-1-yl]-methanone

A mixture of the title D compound, (2-allyloxy-4-nitrophenyl)-[octahydro-1(2H)-quinolin-1-yl]-methanone (1.15 g, 3.43 mmol) and 10% Pd/C (50 mg) in a mixture of 15 mL of EtOAc and 15 mL of EtOH is hydrogenated under 1 atm hydrogen at RT for 16 h. The catalyst is removed by vacuum filtration through celite. The residue is purified by flash chromatography (50% EtOAc in hexane) to afford (4-amino-2-propoxy-phenyl)-[octahydro-1(2H)-quinolin-1-yl]-methanone as a yellow foam: NMR (DMSO-d₆) 0.95 (t, 3H, J=7.3), 1.15-1.72 (m, 15H), 3.20 (br s, 3H), 3.80 (t, 2H, J=6.4), 5.31 (br s, 2H), 6.10-6.17 (m, 2H), 6.71-6.79 (m, 1H); API-MS 317 [M+H]⁺.

### F. 2,4-Dichloro-N-[4-(octahydro-1(2H)-quinoline-1-carbonyl)-3-propoxyphenyl]-benzamide

Under multiparallel solution phase synthesis conditions, a solution of NMM (2.0 M in THF, 135 μL, 0.27 mol) and a solution of 2,4-dichlorobenzoyl chloride (1.0 M in THF, 225 μL, 0.225 mmol) are dispensed sequentially into a vial containing a solution of the title E compound, (4-amino-2-propoxyphenyl)-[octahydro-1(2H)-quinolin-1-yl]-methanone (0.60 M in DMF, 250 μL, 0.15 mmol). The vial is shaken at RT for 16 h. A solution of aqueous lithium hydroxide (1.5 N, 100 μL, 0.15 mmol) is dispensed into the vial, and the vial is shaken for 20 min. The reaction mixture is acidified with 50 μL TFA and purified by HPLC to afford 2,4-dichloro-N-[4-(octahydro-1(2H)-quinoline-1-carbonyl)-3-propoxyphenyl]benzamide: API-MS 489 [M+H]⁺.

### EXAMPLE 18

The following compounds are prepared analogously to Example 17 by treating the title E compound in Example 17 with the appropriate N-derivatizing agent, such as an activated derivative of a carboxylic acid or a chloroformate.

| Compd | Structure | MS [m/z] |
|---|---|---|
| 18-1 | 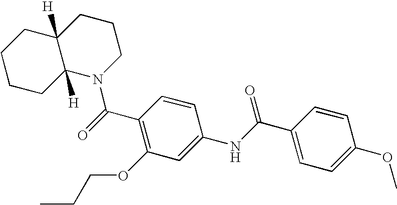 | [M + 1]⁺ 452 |
| 18-2 | 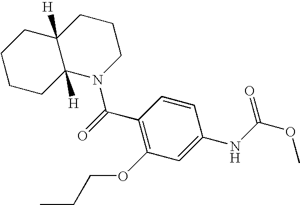 | [M + 1]⁺ 376 |
| 18-3 | 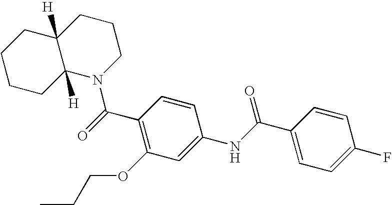 | [M + 1]⁺ 439 |
| 18-4 | 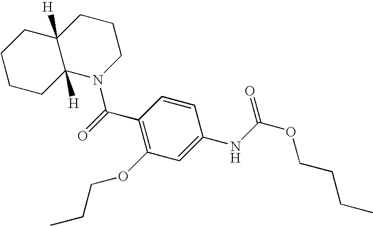 | [M + 1]⁺ 418 |
| 18-5 | 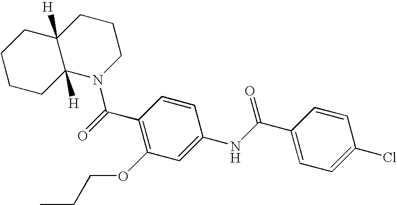 | [M + 1]⁺ 455 |
| 18-6 | 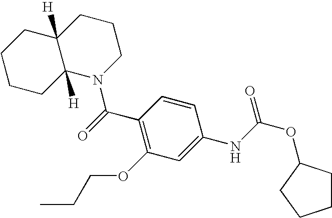 | [M + 1]⁺ 430 |
| 18-7 | 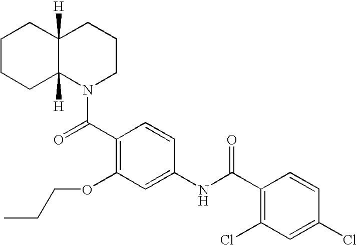 | [M + 1]⁺ 489 |
| 18-8 | 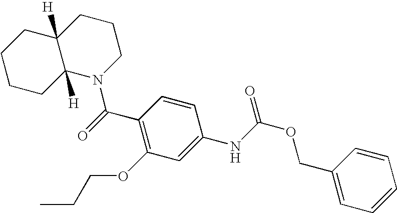 | [M + 1]⁺ 452 |
| 18-9 | 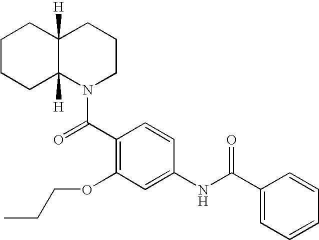 | [M + 1]⁺ 422 |
| 18-10 | 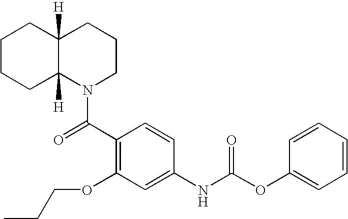 | [M + 1]⁺ 438 |
| 18-11 | 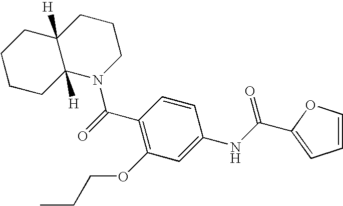 | [M + 1]⁺ 411 |
| 18-12 | 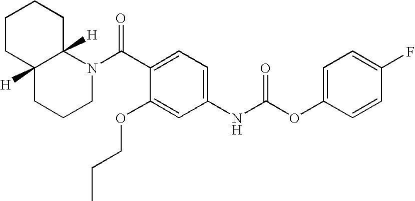 | [M + 1]⁺ 455 |

### EXAMPLE 19

### N-{2-Acetylamino-4-[(4aS*,8aR*)-octahydro-quinoline-1-carbonyl]-phenyl}-2,4-dichloro-benzamide

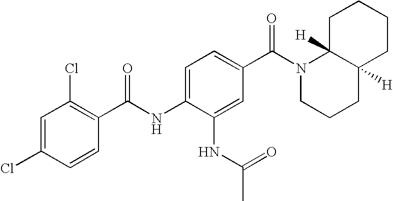

### A. (4-Amino-3-nitrophenyl)-(4aS*,8aR*)-octahydro-1(2H)-quinolin-1-yl-methanone

To a solution of 8.5 g (59.2 mmol) of decahydroquinoline, 10.8 g (59.2 mmol) of 4-amino-3-nitrobenzoic acid, and 8.0 g (59.2 mmol) of 1-hydroxybenzotriazole (HOBt) in 100 mL DMF is added 11.4 g (59.2 mmol) of 1-(3-dimethylaminopropyl)-3-ethylcarbodiimide (EDCI). The mixture is stirred at RT for 18 h, then water is added slowly. The resulting precipitate is filtered, washed with water and dried under vacuum. Recrystallization from methanol (MeOH) gives (4-amino-3-nitrophenyl)-(4aS*,8aR*)-octahydro-1(2H)-quinolin-1-yl-methanone: m.p. 212-215° C.; NMR (DMSO-d₆) 7.97 (s, 1H), 7.69 (s, 2H), 7.43 (d, 1H, J=8.7), 7.03 (d, 1H, J=9.0), 3.52-3.14 (m, 3H), 2.06 (d, 1H, J=12.1), 1.81-0.93 (m, 12H).

### B. 2,4-Dichloro-N-{2-nitro-4-[(4aS*,8aR*)-octahydro-1(2H)-quinoline-1-carbonyl]-phenyl}-benzamide

To a solution of 6.06 g (20 mmol) of the title A compound, (4-amino-3-nitro-phenyl)-(4aS*,8aR*)-octahydro-1(2H)-quinolin-1-yl-methanone and 0.7 g of 4-dimethyl-aminopyridine (DMAP) in 70 mL pyridine is added 4.6 g (21 mmol) of 2,4-dichlorobenzoyl chloride. The mixture is heated at 70° C. for 1 h, then stirred at RT for 18 h. An additional 2.1 g of acid chloride is added and the reaction mixture is heated at 85° C. for 16 h. Pyridine is removed under reduced pressure to give a thick oil which is dissolved in dichloromethane and washed consecutively with water, 3 N aqueous HCl, and dilute ammonium hydroxide. The organic phase is dried over anhydrous Na₂SO₄, the solvent is removed under reduced pressure, and the residue is flash chromatographed using 1% MeOH in dichloromethane as the eluent to give 2,4-dichloro-N-{2-nitro-4-[(4aS*,8aR*)-octahydro-1(2H)-quinoline-1-carbonyl]-phenyl}-benzamide. An analytical sample is crystallized from diethyl ether/hexane: m.p. 165-166° C.; NMR (CDCl₃) 10.95 (s, 1H), 8.96 (d, 1H, J=8.8), 8.34 (m, 1H), 7.76 (m, 1H), 7.69 (d, 1H, J=8.1), 7.54 (m, 1H), 7.41 (m, 1H), 3.55-3.34 (m, 3H), 2.27 (m, 1H), 1.86-1.04 (m, 12H).

### C. N-{2-Amino-4-[(4aS*,8aR*)-octahydro-1(2H)-quinoline-1-carbonyl}-phenyl}-2,4-dichloro-benzamide

A mixture of 2.4 g (5 mmol) of the title B compound, 2,4-dichloro-N-{2-nitro-4-[(4aS*,8aR*)-octahydro-1(2H)-quinoline-1 carbonyl]-phenyl}benzamide and 0.7 g of 5% platinum on carbon (sulfided) in 150 mL of MeOH/dichloromethane (1:1) is hydrogenated at 50 psi for 3 h. The catalyst is removed by filtration and the solvent is removed under reduced pressure to give a foam. Recrystallization from diethyl ether gives N-{2-amino-4-[(4aS*,8aR*)-octahydro-1(2H)-quinoline-1-carbonyl}phenyl}-2,4-dichloro-benzamide: mp 208-210° C.; NMR (CDCl₃) 9.56 (s, 1H), 7.67 (d, 1H, J=8.3), 7.46 (m, 1H), 7.35 (m, 1H), 7.03 (d, 1H, J=8.3), 6.66 (s, 1H), 6.55 (m, 1H), 3.39-3.15 (m, 3H), 1.98 (m, 1H), 1.84-0.97 (m, 14H).

### D. N-{2-Acetylamino-4-[(4aS*,8aR*)-octahydro-1(2H)-quinoline-1-carbonyl]-phenyl}-2,4-dichloro-benzamide

To a solution of 178 mg (0.4 mmol) of the title C compound, N-{2-amino-4-[(4aS*,8aR*)-octahydro-1(2H)-quinoline-1-carbonyl}phenyl}-2,4-dichloro-benzamide and 111 mg (1.1 mmol) of triethylamine in 4 mL of dichloromethane is added 102 mg (1.0 mmol) of acetic anhydride. The mixture is stirred at RT for 18 h, then washed with water. The organic phase is dried over sodium sulfate and the solvent is removed under reduced pressure. The residue is flash chromatographed using 2% MeOH in dichloromethane as the eluent. The product is crystallized from diethyl ether to give N-{2-acetylamino-4-[(4aS*,8aR*)-octahydro-1 (2H)-quinoline-1-carbonyl]-phenyl}-2,4-dichloro-benzamide: m.p. 187-188° C.; NMR (CDCl₃) 9.96 (s, 1H), 8.53 (s, 1H), 7.63 (d, 1H, J=8.3), 7.48 (m, 2H), 7.37 (m, 1H), 7.27 (m, 1H), 6.87 (m, 1H), 3.33-3.17 (m, 3H), 2.17 (s, 3H), 2.00 (m, 1H), 1.82-0.99 (m, 14H).

### EXAMPLE 20

### N-{2-Benzoylamino-4-[(4aS*,8aR*)-octahydro-1(2H)-quinoline-1-carbonyl]-phenyl}-2,4-dichloro-benzamide

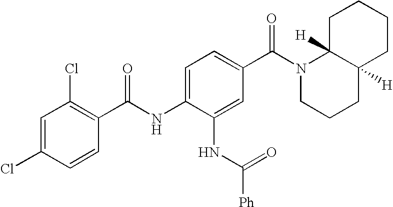

The title compound is prepared analogously to Example 19: API-MS 551 [M+1]⁺.

### EXAMPLE 21

### (4aS*,8aR*)-Octahydro-quinolin-1-yl-[4-(piperidine-1-carbonyl)-phenyl]-methanone

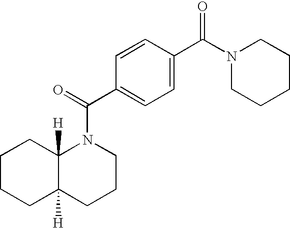

### A. 4-((4aS*,8aR*)-Octahydro-1(2H)-quinoline-1-carbonyl)-benzoic acid methyl ester

To a solution of 2.8 g (20 mmol) of decahydroquinoline and 2.25 g (22 mmol) of triethylamine in 100 mL of dichloromethane is added dropwise a solution of 4.0 g (20 mmol) of 4-carbomethoxybenzoyl chloride in 10 mL dichloromethane. After stirring the mixture at RT for 18 h, it is washed with 1 N aqueous HCl, 1 N aqueous NaOH, and water. The organic phase is dried over sodium sulfate and the solvent is removed under reduced pressure. The residue is flash chromatographed using hexane/EtOAc (3:2) to afford 4-((4aS*,8aR*)-octahydro-1(2H)-quinoline-1-carbonyl)-benzoic acid methyl ester: m.p. 109-110° C.; NMR (CDCl₃) 8.06 (d, 2H, J=8.4), 7.45 (d, 2H, J=8.4), 3.93 (s, 3H), 3.49 (m, 1H), 3.36-3.30 (m, 2H), 2.28 (m, 1H), 1.87-1.00 (m, 12H).

### B. 4-((4aS*,8aR*)-Octahydro-1(2H)-quinoline-1-carbonyl)-benzoic acid

To a solution of 3.0 g (10 mmol) of the title A compound, 4-((4aS*,8aR*)-octahydro-1(2H)-quinoline-1-carbonyl)-benzoic acid methyl ester in 50 mL of MeOH is added 30 mL (30 mmol) of 1 N aqueous NaOH. After stirring the mixture at RT for 18 h, MeOH is removed under reduced pressure. The aqueous solution is acidified with 1 N aqueous HCl and extracted with EtOAc. The organic phase is dried over sodium sulfate and the solvent is removed under reduced pressure to give 4-((4aS*,8aR*)-octahydro-1(2H)-quinoline-1-carbonyl)-benzoic acid: m.p. 194-195° C.; NMR (CDCl₃) 8.12 (d, 2H, J=8.1), 7.48 (d, 2H, J=8.1), 4.29 (s, 1H, broad), 3.52 (m, 1H), 3.40-3.30 (m, 2H), 2.30 (m, 1H), 1.85-1.00 (m, 12H).

### C. (4aS*,8aR*)-Octahydro-1(2H)-quinolin-1-yl-[4-(piperidine-1-carbonyl)-phenyl]methanone

To a solution of 0.2 g (0.7 mmol) of the title B compound, 4-((4aS*,8aR*)-octahydro-1(2H)-quinoline-1-carbonyl)-benzoic acid, 0.28 g (1.4 mmol) of EDCI and 0.12 g (0.84 mmol) of HOBt in 3 mL of dichloromethane is added 0.1 g (0.7 mmol) of piperidine and the resulting mixture is stirred at RT for 18 h. EtOAc is added and the mixture is washed with 1 N aqueous HCl. The organic phase is dried over anhydrous magnesium sulfate (MgSO₄) and the solvent is removed under reduced pressure. The residue is flash chromatographed using EtOAc as the eluent to give (4aS*,8aR*)-octahydro-1(2H)-quinolin-1-yl-[4-(piperidine-1-carbonyl)-phenyl]-methanone as a white solid: m.p. 136-137° C.; NMR (CDCl₃Y 7.41 (s, 4H), 3.71 (m, 2H, broad), 3.52 (m, 1H), 3.40-3.26 (m, 4H), 2.28 (m, 1H), 1.84-1.02 (m, 18H).

### EXAMPLE 22

### 4-[(4aS*,8aR*)-Octahydro-1(2H)-quinoline-1-carbonyl]-N-p-tolylbenzamide

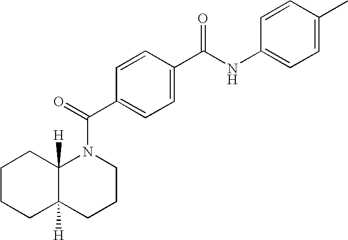

### A. 4-[(4aS*,8aR*)-Octahydro-1(2H)-quinoline-1-carbonyl]-benzoyl chloride

To a solution of 100 mg (0.35 mmol) of the title B compound in Example 21, 4-[(4aS*,8aR*)-octahydro-1(2H)-quinoline-1-carbonyl]-benzoic acid in fib mL of dichloromethane is added 178 mg (1.4 mmol) of oxalyl chloride and one drop of DMF. The mixture is stirred at RT for 18 h, then the solvent is removed under reduced pressure. Dichloromethane is added to the residue and the solvent is removed under reduced pressure. This is repeated three times. The resulting material, 4-[(4aS*,8aR*)-octahydro-1(2H)-quinoline-1-carbonyl]-benzoyl chloride is used directly in the next reaction.

### B. 4-[(4aS*,8aR*)-Octahydro-1(2H)-quinoline-1-carbonyl]-N-p-tolylbenzamide

To a solution of the title A compound, 4-[(4 aS*,8aR*)-octahydro-1(2H)-quinoline-1-carbonyl]-benzoyl chloride and 94 mg (0.7 mmol) of diisopropylethylamine in 10 mL of dichloromethane is added 38 mg (0.35 mmol) of p-toluidine. The mixture is stirred at RT for 18 h, then the solvent is removed under reduced pressure. The residue is flash chromatographed using dichloromethane/EtOH (69:4) to give 4-[(4aS*,8aR*)-octahydro-1(2H)-quinoline-1-carbonyl]-N-p-tolylbenzamide: m.p. 199-203° C.; NMR (CDCl₃) 8.43 (s, 1H), 7.83 (d, J=8.1, 2H), 7.61 (d, J=8.4, 2H), 7.34 (d, J=8.1, 2H), 7.17 (d, J=8.4, 2H), 3.51 (m, 1H), 3.39-3.27 (m, 2H), 2.35 (s, 3H), 2.27 (m, 1H), 1.87-1.54 (m, 6H), 1.50-0.99 (m, 6H).

### EXAMPLE 23

The following compounds are prepared analogously to Examples 21 and 22 by reacting the title B compound in Example 21 or the title A compound in Example 22, respectively, with the appropriate amine.

| Compd | Structure | MS [m/z] |
|---|---|---|
| 23-1 | 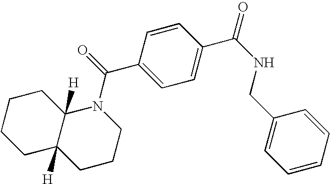 | [M + 1]⁺ 377 |
| 23-2 | 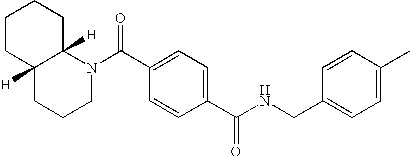 | [M + 1]⁺ 391 |
| 23-3 | 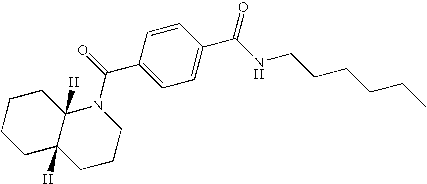 | [M + 1]⁺ 371 |
| 23-4 | 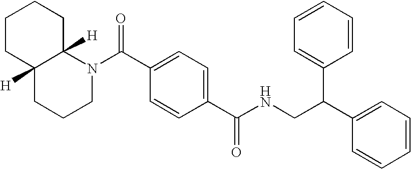 | [M + 1]⁺ 467 |
| 23-5 | 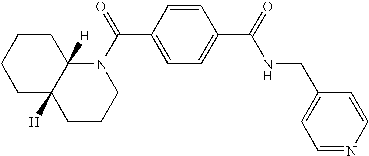 | [M + 1]⁺ 378 |
| 23-6 | 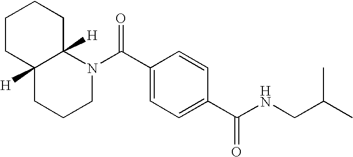 | [M + 1]⁺ 343 |
| 23-7 | 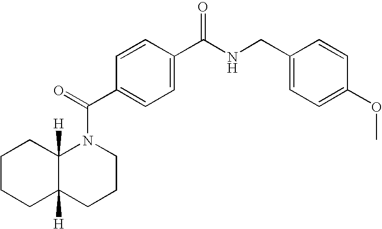 | [M + 1]⁺ 407 |
| 23-8 | 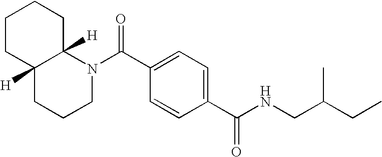 | [M + 1]⁺ 357 |
| 23-9 | 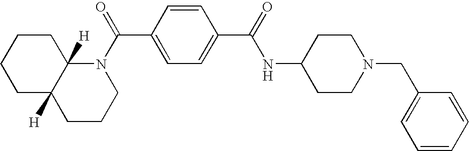 | [M + 1]⁺ 460 |
| 23-10 | 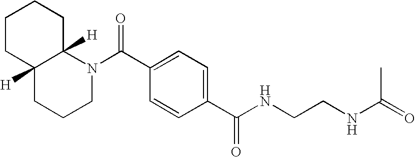 | [M + 1]⁺ 372 |
| 23-11 | 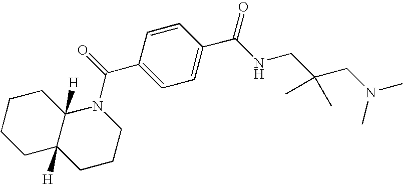 | [M + 1]⁺ 400 |
| 23-12 | 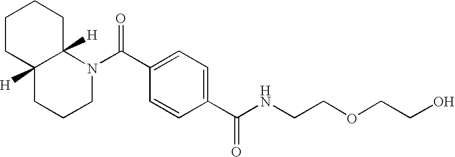 | [M + 1]⁺ 375 |
| 23-13 | 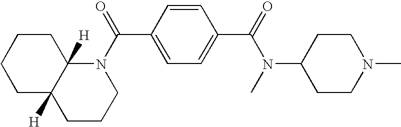 | [M + 1]⁺ 398 |
| 23-14 | 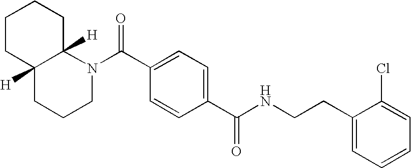 | [M + 1]⁺ 425 |
| 23-15 | 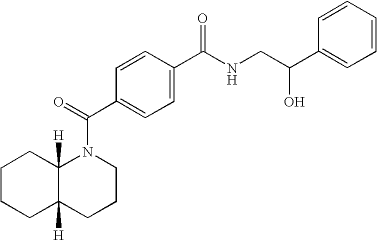 | [M + 1]⁺ 407 |
| 23-16 | 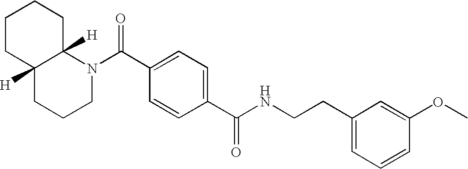 | [M + 1]⁺ 421 |
| 23-17 | 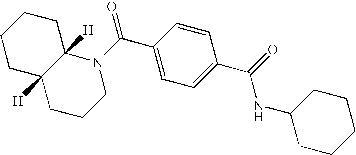 | [M + 1]⁺ 369 |
| 23-18 | 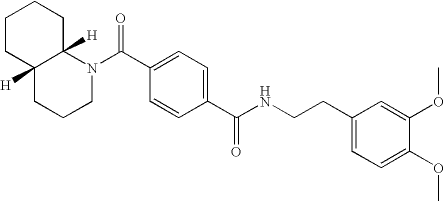 | [M + 1]⁺ 451 |
| 23-19 | 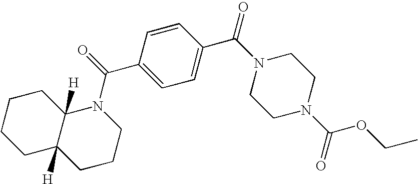 | [M + 1]⁺ 428 |
| 23-20 | 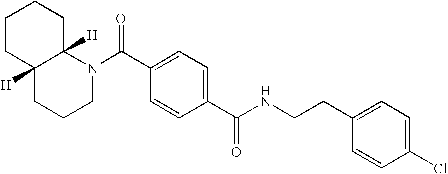 | [M + 1]⁺ 425 |
| 23-21 | 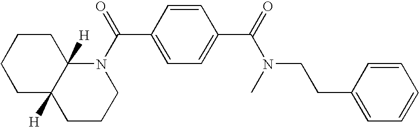 | [M + 1]⁺ 405 |
| 23-22 | 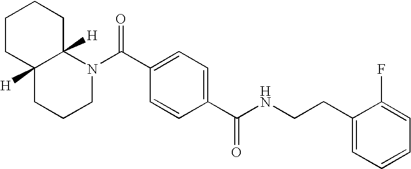 | [M + 1]⁺ 409 |
| 23-23 | 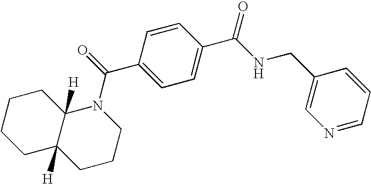 | [M + 1]⁺ 378 |
| 23-24 | 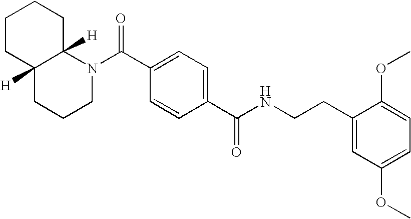 | [M + 1]⁺ 451 |
| 23-25 | 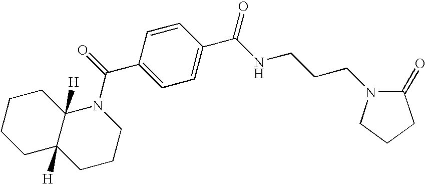 | [M + 1]⁺ 412 |
| 23-26 | 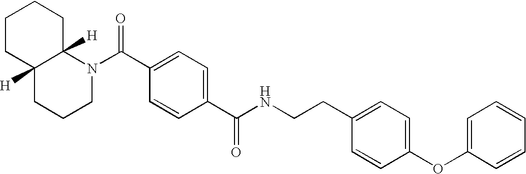 | [M + 1]⁺ 483 |
| 23-27 | 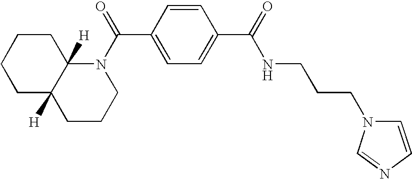 | [M + 1]⁺ 395 |
| 23-28 | 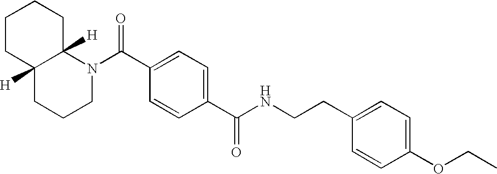 | [M + 1]⁺ 435 |
| 23-29 | 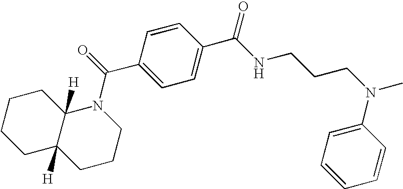 | [M + 1]⁺ 434 |
| 23-30 | 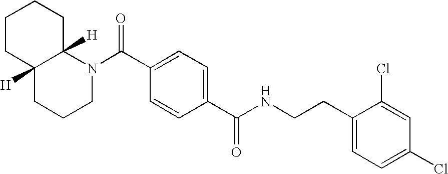 | [M + 1]⁺ 459 |
| 23-31 | 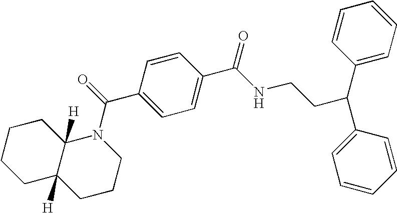 | [M + 1]⁺ 481 |
| 23-32 | 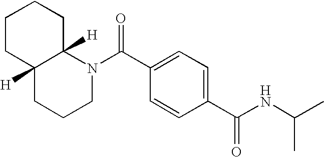 | [M + 1]⁺ 329 |
| 23-33 | 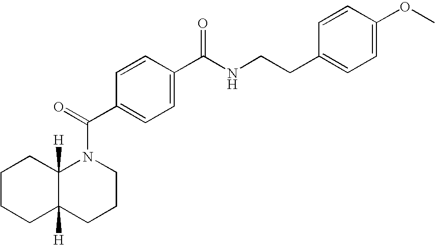 | [M + 1]⁺ 421 |
| 23-34 | 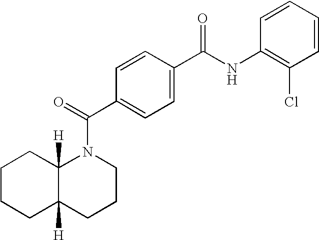 | [M + 1]⁺ 397 |
| 23-35 | 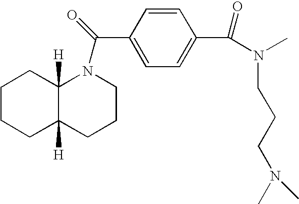 | [M + 1]⁺ 386 |
| 23-36 | 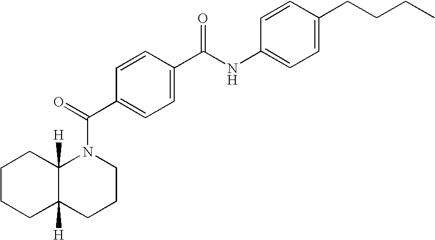 | [M + 1]⁺ 419 |
| 23-37 | 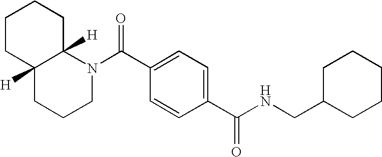 | [M + 1]⁺ 383 |
| 23-38 | 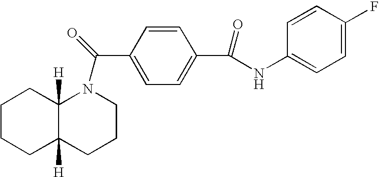 | [M + 1]⁺ 381 |
| 23-39 | 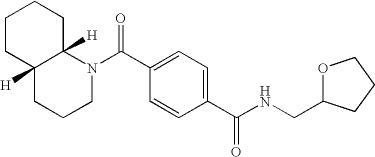 | [M + 1]⁺ 371 |
| 23-40 | 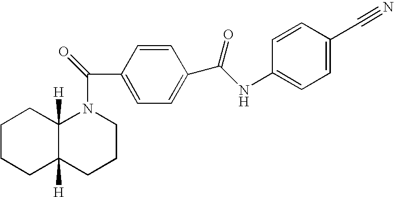 | [M + 1]⁺ 388 |
| 23-41 | 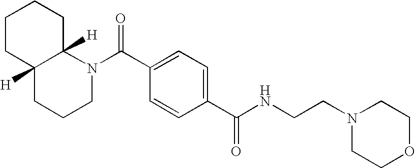 | [M + 1]⁺ 400 |
| 23-42 | 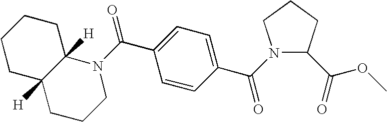 | [M + 1]⁺ 399 |
| 23-43 | 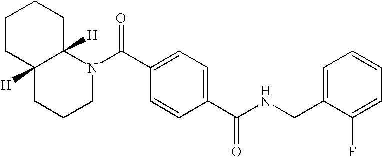 | [M + 1]⁺ 395 |
| 23-44 | 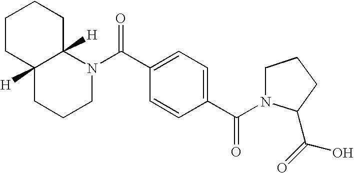 | [M − 1]⁻ 383 |
| 23-45 | 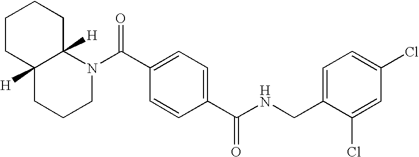 | [M + 1]⁺ 445 |
| 23-46 | 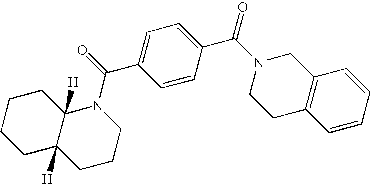 | [M + 1]⁺ 403 |
| 23-47 | 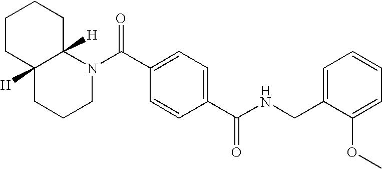 | [M + 1]⁺ 407 |
| 23-48 | 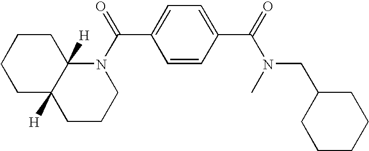 | [M + 1]⁺ 397 |
| 23-49 | 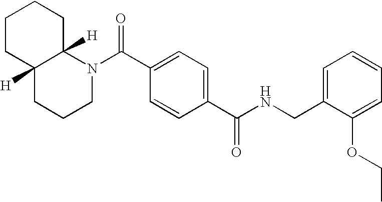 | [M + 1]⁺ 421 |
| 23-50 | 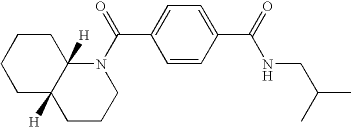 | [M + 1]⁺ 343 |
| 23-51 | 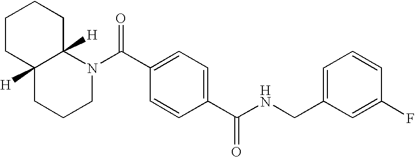 | [M + 1]⁺ 395 |
| 23-52 | 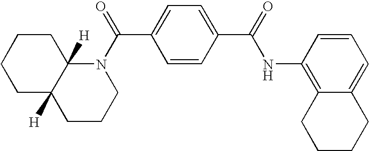 | [M + 1]⁺ 417 |
| 23-53 | 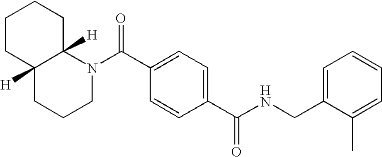 | [M + 1]⁺ 391 |
| 23-54 | 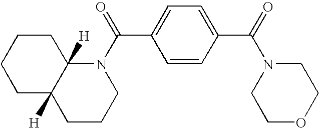 | [M + 1]⁺ 357 |
| 23-55 | 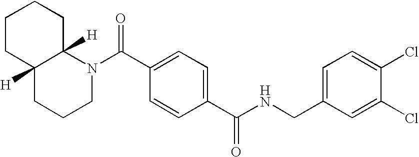 | [M + 1]⁺ 445 |
| 23-56 | 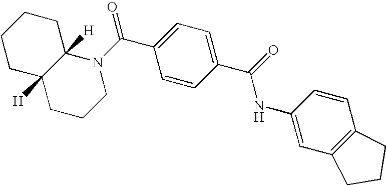 | [M + 1]⁺ 403 |
| 23-57 | 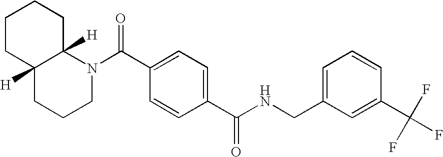 | [M + 1]⁺ 445 |
| 23-58 | 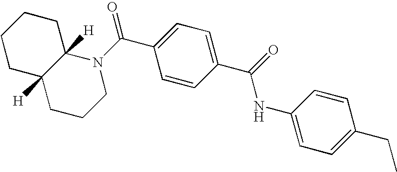 | [M + 1]⁺ 391 |
| 23-59 | 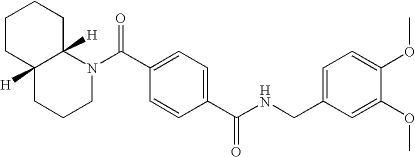 | [M + 1]⁺ 437 |
| 23-60 | 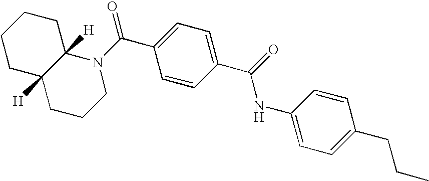 | [M + 1]⁺ 405 |
| 23-61 | 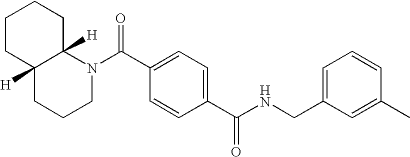 | [M + 1]⁺ 391 |
| 23-62 | 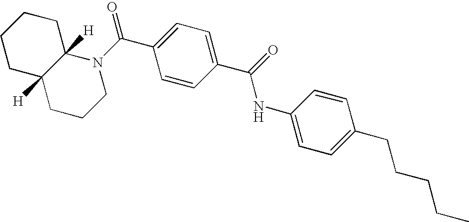 | [M + 1]⁺ 433 |
| 23-63 | 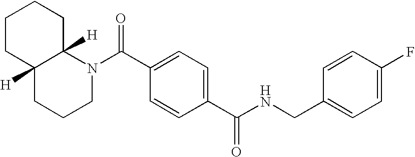 | [M + 1]⁺ 395 |
| 23-64 | 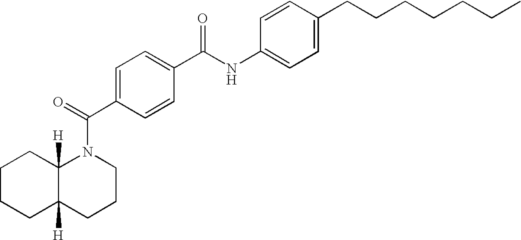 | [M + 1]⁺ 461 |
| 23-65 | 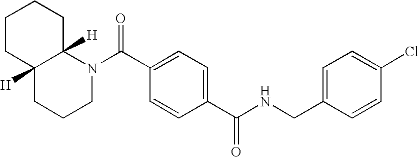 | [M + 1]⁺ 411 |
| 23-66 | 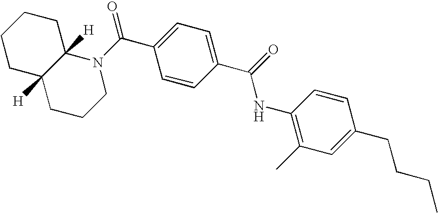 | [M + 1]⁺ 433 |

### EXAMPLE 24

### 2-Acetylamino-N-isobutyl-4-(4aS*,8aR*)-octahydro-1(2H)-quinoline-1-carbonyl]-benzamide

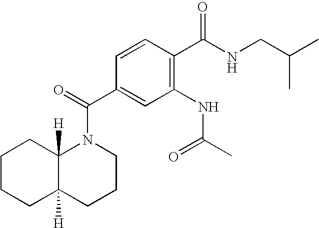

### A. 2-Nitroterephthalic acid 4-methyl ester

To a solution of 80 g (379 mmol) of 2-nitroterephthalic acid in 400 mL of MeOH is slowly added 40 mL of concentrated sulfuric acid. The mixture is refluxed for 1 h, then cooled to RT. Water is slowly added until crystallization occurred. The resulting solid is filtered, washed with water and dried to give 2-nitroterephthalic acid 4-methyl ester: NMR (CDCl₃) 9.22 (s, broad, 1H), 8.55 (m, 1H), 8.37 (dd, 1H), 7.96 (d, J=7.9, 1H), 4.02 (s, 3H).

### B. 4-Chlorocarbonyl-3-nitrobenzoic acid methyl ester

A mixture of 30.0 g (119 mmol) of the title A compound, 2-nitroterephthalic acid 4-methyl ester in 45 mL of thionyl chloride is refluxed for 1 h. The excess thionyl chloride is removed under reduced pressure and the crude 4-chlorocarbonyl-3-nitrobenzoic acid methyl ester is used as such in the next step.

### C. N-Isobutyl-3-nitroterephthalamic acid methyl ester

To a solution of 29.4 g (402 mmol) of isobutylamine in 500 mL of dichloromethane at 10° C. is added dropwise a solution of the title B compound, 4-chlorocarbonyl-3-nitrobenzoic acid methyl ester in 100 mL of dichloromethane. The mixture is allowed to warm to room temperature, then washed with water. The organic phase is washed with 1 N aqueous HCl and dried over anhydrous Na₂SO₄. The solvent is removed under reduced pressure and the residual solid is crystallized from diethyl ether/hexane to give N-isobutyl-3-nitroterephthalamic acid methyl ester: m.p. 90-91° C.; NMR (CDCl₃) 8.65 (s, 1H), 8.29 (dd, 1H), 7.59 (d, J=7.9, 1H), 3.99 (s, 3H), 3.30 (m, 2H), 1.95 (m, 1H), 1.00 (d, J=6.8, 6H).

### D. N-Isobutyl-3-nitroterephthalamic acid

To a solution of 14.0 g (50 mmol) of the title C compound, N-isobutyl-3-nitro-terephthalamic acid methyl ester in 100 mL of MeOH is added 60.0 mL of 1 N aqueous NaOH. After stirring the mixture at RT for 18 h, the mixture is cooled in an ice bath and 21 mL of 3 N aqueous HCl is added. The resulting solid is filtered, washed with water and dried to give N-isobutyl-3-nitroterephthalamic acid: m.p. 255-257° C.; NMR (DMSO-d₆) 8.78 (m, 1H), 8.43 (s, 1H), 8.27 (dd, 1H), 7.71 (d, J=7.9, 1H), 3.06 (m, 2H), 1.82 (m, 1H), 0.91 (d, J=6.8, 6H).

### E. N-Isobutyl-2-nitro-4-[(4aS*,8aR*)-octahydro-1(2H)-quinoline-1-carbonyl]-benzamide

To a solution of 1.06 g (4 mmol) of the title D compound, N-isobutyl-3-nitro-terephthalamic acid, 570 mg (4.2 mmol) of HOBt, and 810 mg (4.2 mmol) of EDCI in 10 mL of DMF is added 575 mg (4.0 mmol) of decahydroquinoline. After the mixture is stirred at RT for 18 h, water is added. The resulting precipitate is filtered, washed with water and dried to give N-isobutyl-2-nitro-4-[(4aS*,8aR*)-octahydro-1(2H)-quinoline-1-carbonyl]-benzamide: m.p. 127-129° C.; NMR (CDCl₃) 8.00 (s, 1H), 7.62 (dd, 1H), 7.52 (d, J=7.7, 1H), 6.25 (m, 1H), 3.55-3.22 (m, 5H), 2.25 (m, 1H), 1.96 (m, 1H), 1.89-1.59 (m, 7H), 1.51-1.00 (m, 5-H), 1.01 (d, J=6.6, 6H).

### F. 2-Amino-N-isobutyl-4-[(4aS*,8aR*)-octahydro-quinoline-1-carbonyl]-benzamide

A solution of 650 mg (1.7 mmol) of the title E compound, N-isobutyl-2-nitro-4-[(4aS*,8aR*)-octahydro-1(2H)-quinoline-1-carbonyl]-benzamide in 25 mL of EtOH is hydrogenated over 100 mg of 5% Pd/C at 50 psi for 16 h. The catalyst is filtered through Celite and the solvent is removed under reduced pressure. The resulting foam is dissolved in dichloromethane and washed with dilute ammonium hydroxide. The organic phase is dried over anhydrous Na₂SO₄ and the solvent is removed under reduced pressure. The residual solid is crystallized from diethyl ether to give 2-amino-N-isobutyl-4-[(4aS*,8aR*)-octahydro-quinoline-1-carbonyl]-benzamide: m.p. 153-155° C.; NMR (CDCl₃) 7.30 (d, J=7.9, 11H), 6.67-6.56 (m, 2H), 6.15 (s, 1H), 5.54 (s, 2H), 3.51-3.26 (m, 2H), 3.24 (M, 2H), 2.27 (m, 1H), 1.89 (m, 1H), 1.85-1.00 (m, 13H), 0.98 (d, J=6.8, 6H).

### G. 2-Acetylamino-N-isobutyl-4-(4aS*,8aR*)-octahydro-1(2H)-quinoline-1-carbonyl]-benzamide

To a solution of 125 mg (0.35 mmol) of the title F compound, 2-amino-N-isobutyl-4-[(4aS*,8aR*)-octahydro-quinoline-1-carbonyl]-benzamide and 71 mg (0.7 mmol) of triethylamine in 3 mL of dichloromethane is added 70 mg (0.68 mmol) of acetic anhydride. The mixture is stirred at RT for 18 h, then washed with water. The organic phase is dried over anhydrous Na₂SO₄ and the solvent is removed under reduced pressure. The residual solid is crystallized from diethyl ether/hexane to give 2-acetylamino-N-isobutyl-4-[(4aS*,8aR*)-octahydro-1(2H)-quinoline-1-carbonyl]-benzamide: m.p. 109-111° C.; NMR (CDCl₃) 11.09 (s, 1H), 8.57 (s, 1H), 7.49 (d, J=7.9, 1H), 7.01 (m, 1H), 6.96 (dd, 1H), 3.49 (m, 1H), 3.40-3.30 (m, 1H), 3.26 (m, 2H), 2.28 (m, 1H), 2.18 (s, 3H), 1.95 (m, 1H), 1.87-1.06 (m, 13H), 1.00 (d, J=6.8, 6H).

### EXAMPLE 25

### 2,4-Dichloro-N-{5-[(4aS*,8aR*)-octahydro-1(2H)-quinoline-1-carbonyl]-pyridin-2-yl}-benzamide

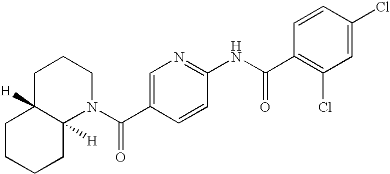

### A. Methyl 6-aminonicotinate hydrochloride

Thionyl chloride is added dropwsie to a stirred suspension of 6-aminonicotinic acid (7.5 g, 54.3 mmol) in MeOH (100 mL) at 50° C. After addition, the reaction mixture is refluxed for 2 h, cooled and then stirred at RT for 16 h. The reaction mixture is concentrated under reduced pressure and the solid residue is triturated with diethyl ether, filtered to give methyl 6-aminonicotinate hydrochloride as a white solid: m.p. 183-185° C.; NMR (MeOH-d₄) 3.98 (3H, S), 7.09 (1H, d), 8.38 (1H, dd), 8.51 (1H, d).

### B. 6-(2,4-Dichlorobenzoylamino)-nicotinic acid methyl ester

To a stirred solution of the title A compound, methyl 6-aminonicotinate hydrochloride (1 g, 5.31 mmol) in dichloromethane (20 mL) at 0° C. is added triethylamine (1.4 g, 13.3 mmol) and 2,4-dichlorobenzoyl chloride (1.67 g, 7.97 mmol). The mixture is stirred at RT for 4 h, diluted with diethyl ether (50 mL), filtered and concentrated under reduced pressure. The residue is purified by flash chromatography on silica (33% EtOAc in hexane) to provide 6-(2,4-dichlorobenzoylamino)-nicotinic acid methyl ester: API-MS 325 [M+1]⁺.

### C. 6-(2,4-Dichlorobenzoylamino)-nicotinic acid

A solution of 4 N aqueous NaOH (3 mL, 12 mmol) is added to a stirred solution of the title B compound, 6-(2,4-dichlorobenzoylamino)-nicotinic acid methyl ester (1.2 g, 3.68 mmol) in a mixture of THF (4 mL) and MeOH (2 mL). After stirring for 10 h, the reaction mixture is poured in 25 mL of water, then extracted successively with diethyl ether. The aqueous layer is acidified with 6 N aqueous HCl, and the product is taken up in EtOAc, dried over anhydrous MgSO₄, and concentrated under reduced pressure. The resulting solid is collected and dried under high vaccum to give 6-(2,4-dichlorobenzoylamino)-nicotinic acid: NMR (DMSO-d₆) 7.48-7.53 (2H, m), 7.63-7.82 (3H, m), 8.31 (2H, q), 8.88 (1H, s); API-MS 311.3 [M+1]⁺, 309.5 [M−1]⁻.

### D. 2,4-Dichloro-N-{2-[(4aS*,8aR*)-octahydro-1(2H)-quinoline-1-carbonyl]-pyridin-2-yl}-benzamide

To a stirred solution of the title C compound, 6-(2,4-dichlorobenzoylamino)-nicotinic acid (1.1 g, 3.54 mmol) in dichloromethane (25 mL) is added decahydroquinoline (545 mg, 3.9 mmol) followed by DMAP (50 mg, 0.41 mmol). The reaction mixture is stirred at RT and EDCI (1.2 g, 6.28 mmol) is added. After stirring overnight at RT, the reaction mixture is poured into water, then extracted with EtOAc. The combined organic extracts are washed successively with 1 N aqueous HCl, water, saturated aqueous NaHCO₃ solution, water and brine. The organic layer is dried over anhydrous Na₂SO₄ and concentrated under reduced pressure. The residue is triturated with diethyl ether to give 2,4-dichloro-N-{5-[(4aS*,8aR*)-octahydro-1 (2H)-quinoline-1-carbonyl]-pyridin-2-yl}-benzamide as a white solid: m.p. 202-203° C.; IR (KBr) 2925, 1678, 1613, 1587, 1310, 855, 796; NMR (CDCl₃) 1.1-2.0 (3H, m), 2.2-2.3 (1H, m), 3.4-3.56 (2H, m), 7.38 (1H, dd), 7.49 (1H, d), 7.72 (2H, d), 7.81 (2H, dd), 8.38 (1H, s), 8.83 (1H, s); API-MS 432.4 [M+1]⁺, 430.6 [M−1]⁻.

### EXAMPLE 26

### 4-Fluoro-N-{5-[(4aS*,8aR*)-octahydro-1(2H)-quinoline-1-carbonyl]-pyridin-2-yl}-benzamide

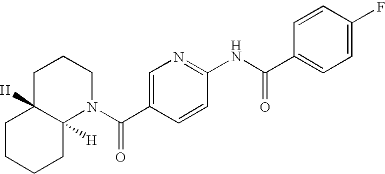

The title compound is prepared analogously to Example 25: m.p. 144-145° C.; API-MS 382 [M+1]⁺.

### EXAMPLE 27

### 3,4-Dimethoxy-N-{4-[(4aS*,8aR*)-octahydro-1(2H)-quinoline-1-carbonyl]-naphthalen-1-yl}-benzamide

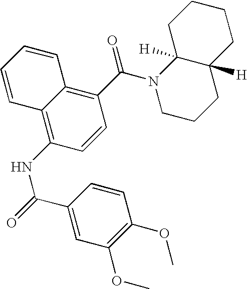

### A. 4-(3,4-Dimethoxy-benzoylamino)-naphthalene-1-carboxylic acid ethyl ester

To a solution of 0.31 g (1.0 mmol) of methanesulfonic acid salt of 4-amino-1-naphthalene carboxylic acid ethyl ester (prepared according to method in Chem. Pharm. Bull., Vol. 32, No. 10, p. 3977 (1984)) and 0.28 g (2.2 mmol) of diisopropylethylamine in 30 mL of 1,2-dichloroethane is added 0.20 g (1.0 mmol) of 3,4-dimethoxybenzoyl chloride while stirring at RT under nitrogen. The mixture is stirred at reflux for 20 h. The solution is cooled to RT and concentrated in vacuo to an oil. The oil is stirred with water and diethyl ether until crystallization takes place. The solid is collected and dried to give 4-[(3,4-dimethoxy-benzoyl)amino]-1-naphthalene carboxylic acid ethyl ester: m.p. 144-146° C.; elemental analysis C₂H₂₁NO₅; Theory: C, 69.64; H, 5.58; N, 3.69. Found: C, 69.36; H, 5.40; N, 3.60. IR (KBr) ester C=0, 1710; amide C=0 1650; API-MS 380 [M+1]⁺, 378 [M−1]⁻; NMR (DMSO-d₆): 1.40 (t, 3H)Y, 3.87 (3, 6H), 4.43 (q, 2H), 7.13 (d, 1H), 7.73 (m, 5H), 8.13 (d, 1H), 8.20 (d, 1H), 8.85 (d, 1H), 10.50 (s, 1H).

### B. 4-[(3,4-dimethoxybenzoyl)amino]-1-naphthalene carboxylic acid

To a suspension of 0.10 g (0.26 mmol) of the title A compound, 4-[(3,4-dimethoxy-benzoyl)amino]-1-naphthalene carboxylic acid ethyl ester in 3 mL of water and 3 mL of EtOH is added 0.3 mL (0.30 mmol) of 1 N aqueous NaOH dropwise while stirring at RT under nitrogen. The suspension is stirred at RT for 30 min and heated at 80° C. for 1 minute. The resulting solution is cooled to RT and the suspension which forms is concentrated. The concentrate is partitioned between 5 mL of water and 5 mL of dichloromethane. The aqueous layer is separated and made acidic by the addition of 1 N aqueous HCl. The precipitate is collected by filtration, washed with water and dried to give 4-[(3,4-dimethoxy-benzoyl)amino]-1-naphthalene carboxylic acid: m.p. 256-259° C.; elemental analysis C₂₀H₁₇NO₅; Theory: C, 68.37; H, 4.88; N, 3.99. Found: C, 68.36; H, 5.05; N, 3.96. IR (KBr) 1682, 1646; API-MS 351.9 [M+H]⁺, 350.0 [M−H]⁻; NMR (DMSO-d₆) 3.87 (s, 6H), 7.13 (d, 1H), 7.70 (m, 5H), 8.11 (d, 1H), 8.21 (d, 1H), 8.97 (d, 1H), 10.49 (s; 1H), 13.10 (broad s, 1H).

### C. 4-(3,4-Dimethoxy-benzoylamino)-naphthalene-1-carbonyl chloride

To a suspension of 0.15 g (0.43 rhmol) of the title B compound, 4-[(3,4-dimethoxy-benzoyl)amino]-1-naphthalene carboxylic acid and 10 mL of anhydrous toluene is added 0.0007 g (0.65 mmol) of thionyl chloride while stirring at RT under nitrogen. The mixture is stirred at 45-55° C. for 20 h. After cooling to RT, the precipitate is collected by filtration, washed with toluene and cyclohexane and dried to give 4-(3,4-dimethoxybenzoylamino)-naphthalene-1-carbonyl chloride: m.p. 167-171° C.; NMR (DMSO-d₆): 3.87 (s, 6H), 7.13 (d, 1H), 7.70 (m, 5H), 8.11 (d, 1H), 8.21 (d, 1H), 8.97 (d, 1H), 10.50 (s, 1H).

### D. 3,4-Dimethoxy-N-{4-[(4aS*,8aR*)-octahydro-1(2H)-quinoline-1-carbonyl]-naphthalen-1-yl}-benzamide

To absolution of 0.13 g (0.36 mmol) of the title C compound, 4-[(3,4-dimethoxy-benzoyl)amino]-1-naphthalene carbonyl chloride and 0.047 g (0.36 mmol) of diisopropylethylamine in 15 mL of 1,2-dichloroethane is added 0.052 g (0.36 mmol) of decahydroquinoline while stirring at RT under nitrogen. The mixture is stirred for 64 h at RT. The solution is washed with 15 mL of water and the organic layer is dried over anhydrous Na₂SO₄, filtered, and concentrated to give a solid. Recrystallization from acetonitrile gives 3,4-dimethoxy-N-[4-(octahydro-1(2H)-quinoline-1(2H)-carbonyl)-naphthalen-1-yl]-benzamide: mp=251-260° C.; elemental analysis C₂₉H₃₂N₂O₄.0.25H₂O, Theory C, 73.00; H, 6.87; N, 5.87. Found C, 72.90; H, 6.87; N, 5.75. IR (KBr) C=0 1655; API-MS 473 [M+H]⁺; 471 [M−H]⁻; NMR (DMSO-d₆): 0.90-2.00 (broad m, 16H), 3.86 (s, 6H), 7.13 (d, 1H), 7.61 (m, 1H), 7.72 (m, 6H), 8.30 (m, 1H), 10.40 (s, 1H).

### EXMPLE 28

The following compounds are prepared analogously to Example 27.

| | | MS |
|---|---|---|
| Compd | Structure | [m/z] |
| 28-1 | 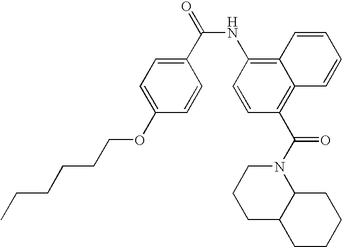 | [M +1]⁺513 |
| 28-2 | 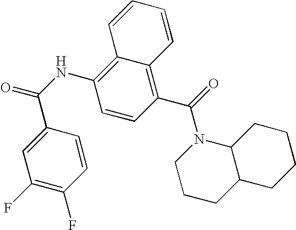 | [M +1]⁺449 |
| 28-3 | 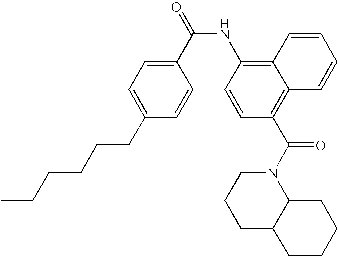 | [M +1]⁺497 |
| 28-4 | 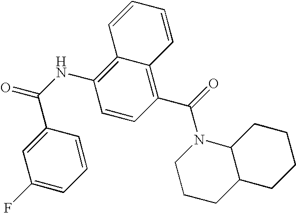 | [M +1]⁺431 |
| 28-5 | 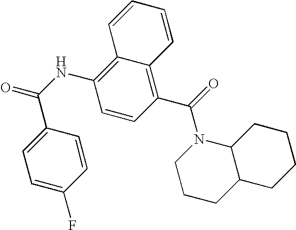 | [M +1]⁺431 |
| 28-6 | 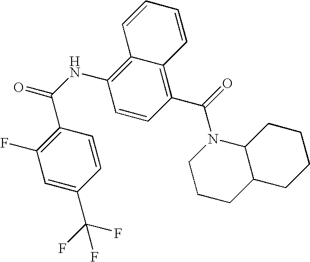 | [M +1]⁺499 |
| 28-7 | 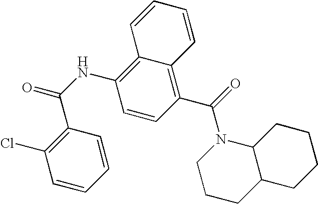 | [M +1]⁺447 |
| 28-8 | 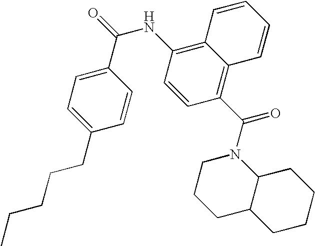 | [M +1]⁺483 |
| 28-9 | 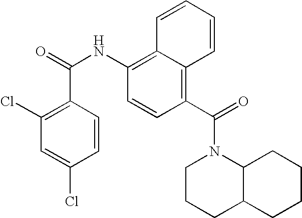 | [M +1]⁺481 |
| 28-10 | 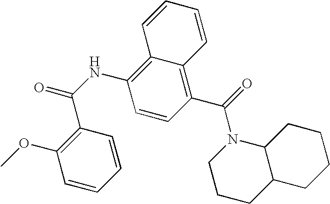 | [M +1]⁺443 |
| 28-11 | 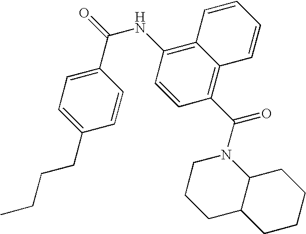 | [M +1]⁺469 |
| 28-12 | 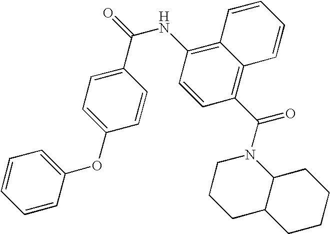 | [M +1]⁺375 |
| 28-13 | 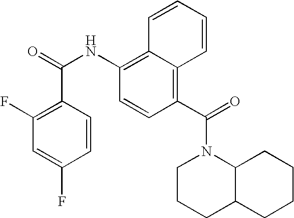 | [M +1]⁺449 |
| 28-14 | 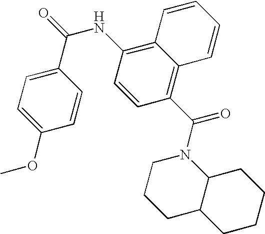 | [M +1]⁺505 |

### EXAMPLE 29

### 4-[(4aS*,8aR*)-Octahydro-1(2H)-quinoline-1-carbonyl]-naphthalene-1-carboxylic acid (4-fluorophenyl)-amide

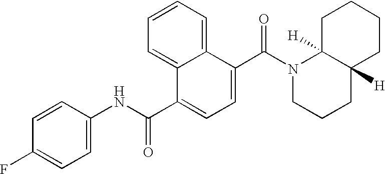

### A. Methyl 1,4-napthalene dicarboxylate

1,4-Naphthalene dicarboxylic acid (12 g, 55.5 mmol) is suspended in 200 mL of MeOH. Hydrogen chloride gas is bubbled through for 10 min and the reaction is refluxed overnight. The resulting mixture is cooled to RT, then concnetrated under reduced pressure. Flash chromatograhpy on silica (eluant: 33% EtOAc in hexane) gives methyl 1,4-napthalene dicarboxylate as a whilte solid: NMR (CDCl₃) 4.01 (6H, s), 7.63 (2H, dd), 8.09 (2H, s), 8.82 (2H, dd).

### B. Methyl 1,4-naphthalene monocarboxylate

NaOH (990 mg, 24.5 mmol) in 5 mL water is added to a stirred solution of the title A compound, methyl 1,4-napthalene dicarboxylate (5.5 g, 22.5 mmol) in MeOH (35 mL). The reaction mixture is refluxed for half an h, then reduced to 1/3 of the volume under reduced pressure. The residue is diluted with 100 mL of water, washed with diethyl ether (2×20 mL), acidified with 2 N aqueous HCl, and extracted with EtOAc. The organic layer is collected, dried over anhydrous Na₂SO₄, and concentrated in vacuo to give methyl 1,4-naphthalene monocarboxylate as a white solid: NMR (DMSO-d₆) 3.98 (3H, s), 7.71 (2H, dd), 8.1 (2H, s), 8.7 (1H, dd), 8.78-8.86 (1H, m).

### C. 4-[(4aS*,8aR*)-Octahydro-1(2H)-quinoline-1-carbonyl]-naphthalene-1-carboxylic acid methyl ester

A solution of the title B compound, methyl 1,4-naphthalene monocarboxylate (2.5 g, 10.9 mmol) in thionyl cholride (15 mL) is stirred and refluxed for 3 h until the reaction mixture is clear. The mixture is concentrated to remove excess of thionyl chloride and the residue is dissolved in dichloromethane (20 mL). The resulting solution is cool to 0° C. and decahydroquinoline (1.5 g, 10.8 mmol) is added, followed by dropwise addition of triethylamine (1.5 mL, 10.9 mmol). After addition, the reaction mixture is allowed to stirred at RT for 1 h. The reaction mixture is poured into water and extracted with EtOAc. The combined organic extracts are washed successively with 1 N aqueous HCl, water, saturated aqueous NaHCO₃, water, dried over anhydrous Na₂SO₄, and concentrated under reduced pressure. Flash chromatography on silica (eluant: 25% EtOAc in hexane) gives 4-[(4aS*,8aR*)-octahydro-1(2H)-quinoline-1-carbonyl]-naphthalene-1-carboxylic acid methyl ester as an oil: NMR (CDCl₃) 1.12-1.93 (12H, m), 2.61-2.92 (1H, m), 3.1-3.29 (1H, m), 3.7-3.79 (2H, m), 4.0 (3H, S), 7.49 (1H, dd), 757.68 (2H, m), 7.86 (1H, dd), 8.12 (1H, d), 8.91 (1H, d).

### D. 4-[(4aS*,8aR*)-Octahydro-1(2H)-quinoline-1-carbonyl]-naphthalene-1-carboxylic acid

To a stirred solution of the title C compound, 4-[(4aS*,8aR*)-octahydro-1(2H)-quinoline-1-carbonyl]-naphthalene-1-carboxylic acid methyl ester (3 g, 8.5 mmol) in 10 mL of MeOH:THF (1:1) is added 2 N aqueous NaOH (5 mL). The reaction mixture is stirred for 3 h, then diluted with water, and washed with diethyl ether. The aqueous layer is collected, acidified with concentrated HCl, then extracted with EtOAc, and the organic solution is dried over anhydrous Na₂SO₄, and concentrated under reduced pressure to give 4-[(4aS*,8aR*)-octahydro-1(2H)-quinoline-1-carbonyl]-naphthalene-1-carboxylic acid as a white solid: NMR (DMSO-d₆) 1.0-1.9 (12H, m), 2.1-2.45 (1H, m), 2.72-3.0 (1H, m), 3.12-3.6 (2H, m), 7.42 (1H, dd), 7.59-7.82 (3H, m), 8.1 (1H, d), 8.82 (1H, d).

### E. 4-[(4aS*,8aR*)-Octahydro-1(2H)-quinoline-1-carbonyl]-naphthalene-1-carboxylic acid (4 fluorophenyl)-amide

A solution of the title D compound, 4-[(4aS*,8aR*)-octahydro-1(2H)-quinoline-1-carbonyl]-naphthalene-1-carboxylic acid (199 mg, 0.59 mmol) in thionyl cholride (1 mL) is stirred and refluxed for 3 h until reaction mixture is clear. The mixture is concentrated to remove excess thionyl chloride and the residue is dissolved in dichloromethane (3 mL). The resulting solution is cool to 0° C. and 4-fluroroaniline (70 mg, 0.63 mmol) is added, followed by dropwise addition of triethylamine (88 μL, 0.63 mmol). After addition, the reaction mixture is allowed to stirred at RT for 4 h. The reaction mixture is poured into water and extracted with EtOAc. The combined organic extracts are washed successively with 1 N aqueous HCl, water, saturated aqueous NaHCO₃, water, and the organic solution is dried over anhydrous Na₂SO₄, and concentrated under reduced pressure. Flash chromatography on silica (eluant: 33% EtOAc in hexane) gives 4-[(4aS*,8aR*)-octahydro-1(2H)-quinoline-1-carbonyl]-naphthalene-1-carboxylic acid (4-fluorophenyl)-amide: NMR (CDCl₃) 1.0-1.13 (2H, M), 1.3-1.78 (8H, m), 1.8-1.92 (2H, m), 2.4-2.48 (1H, m), 2.62-3.12 (3H, m), 3.63 (1H, m), 6.8 (1H, d), 7.03 (2H, t), 7.33-7.5 (3H, m), 7.59-7.7 (1H, m), 7.79-7.88 (2H, m), 8.08-8.15 (1H, m); API-MS 431.5 [M+1]⁺, 429.8 [M−1]⁻.

### EXAMPLE 30

The following compounds are prepared analogously to Example 29.

| Compd | Structure | MS [m/z] |
|---|---|---|
| 30-1 | 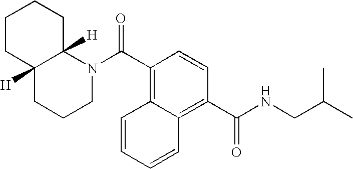 | [M + 1]⁺ 393 |
| 30-2 | 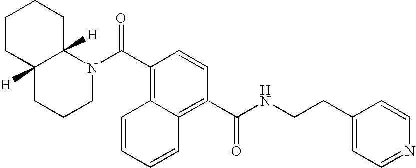 | [M + 1]⁺ 443 |
| 30-3 | 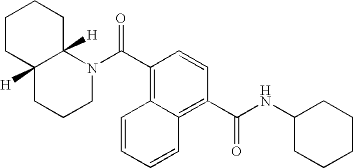 | [M + 1]⁺ 419 |
| 30-4 | 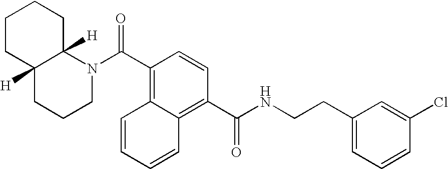 | [M + 1]⁺ 476 |
| 30-5 | 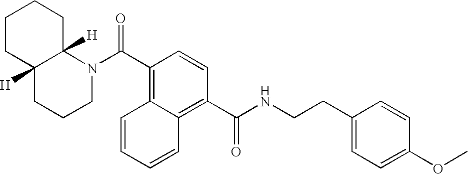 | [M + 1]⁺ 471 |
| 30-6 | 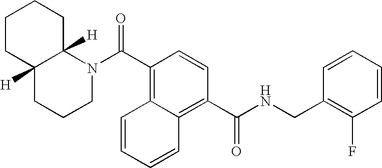 | [M + 1]⁺ 446 |
| 30-7 | 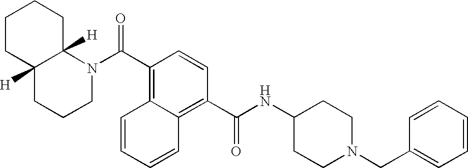 | [M + 1]⁺ 510 |
| 30-8 | 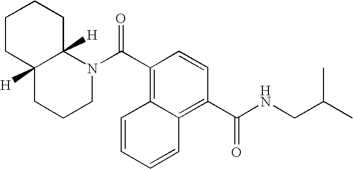 | [M + 1]⁺ 408 |
| 30-9 | 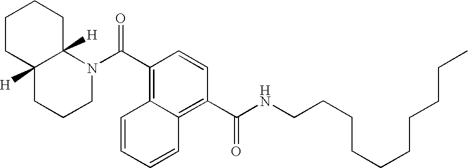 | [M + 1]⁺ 477 |
| 30-10 | 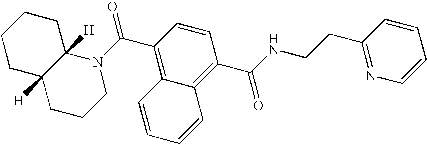 | [M + 1]⁺ 443 |
| 30-11 | 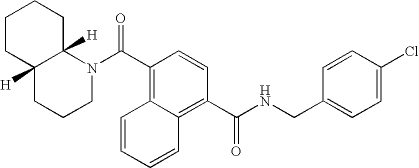 | [M + 1]⁺ 460 |
| 30-12 | 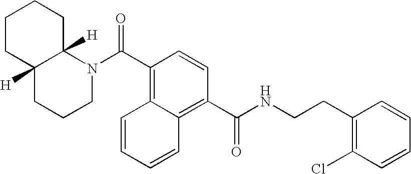 | [M + 1]⁺ 476 |
| 30-13 | 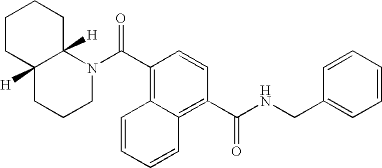 | [M + 1]⁺ 428 |
| 30-14 | 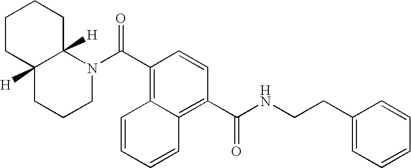 | [M + 1]⁺ 442 |
| 30-15 | 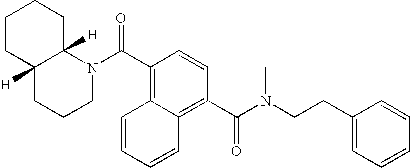 | [M + 1]⁺ 456 |
| 30-16 | 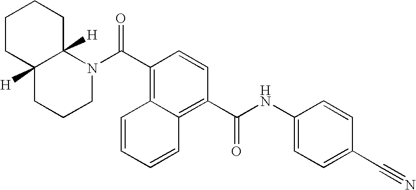 | [M + 1]⁺ 439 |
| 30-17 | 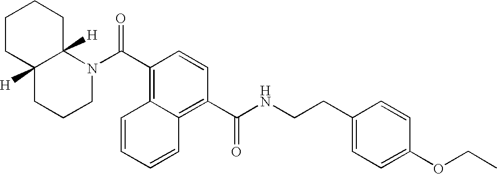 | [M + 1]⁺ 486 |
| 30-18 | 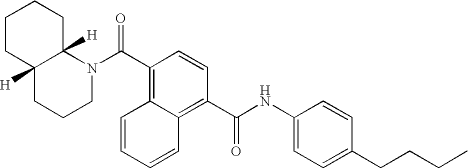 | [M + 1]⁺ 470 |
| 30-19 | 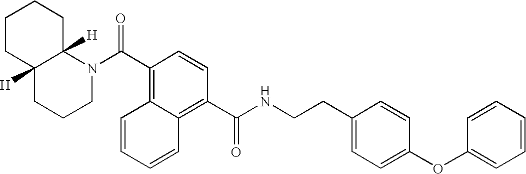 | [M + 1]⁺ 534 |
| 30-20 | 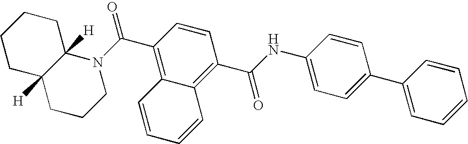 | [M + 1]⁺ 490 |
| 30-21 | 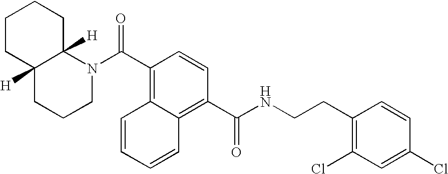 | [M + 1]⁺ 510 |
| 30-22 | 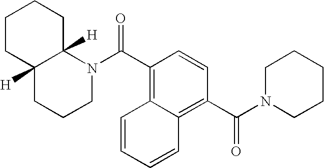 | [M + 1]⁺ 406 |
| 30-23 | 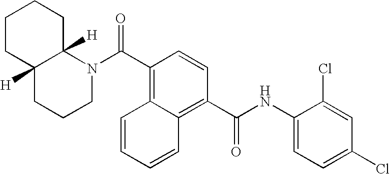 | [M + 1]⁺ 481 |
| 30-24 | 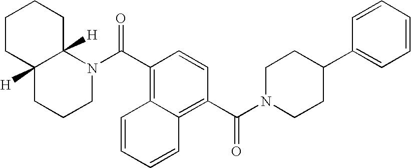 | [M + 1]⁺ 482 |
| 30-25 | 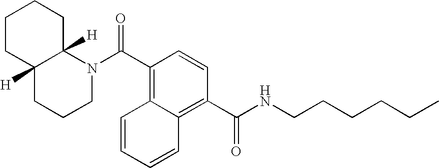 | [M + 1]⁺ 422 |
| 30-26 | 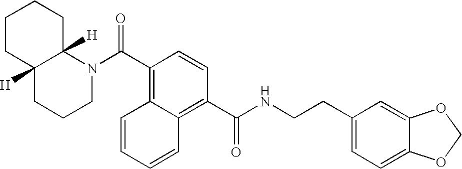 | [M + 1]⁺ 487 |
| 30-27 | 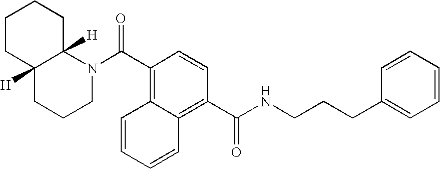 | [M + 1]⁺ 456 |
| 30-28 | 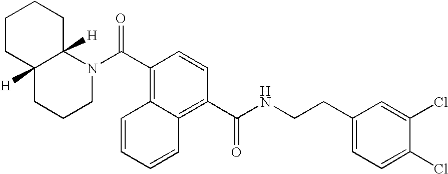 | [M + 1]⁺ 510 |
| 30-29 | 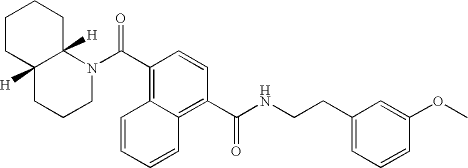 | [M + 1]⁺ 472 |
| 30-30 | 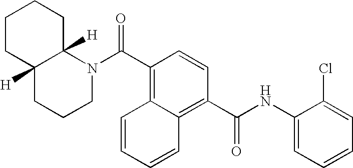 | [M + 1]⁺ 448 |
| 30-31 | 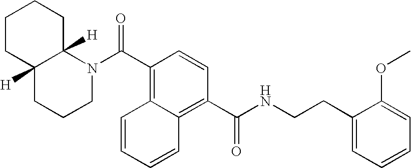 | [M + 1]⁺ 472 |
| 30-32 | 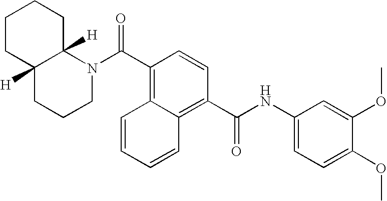 | [M + 1]⁺ 474 |
| 30-33 | 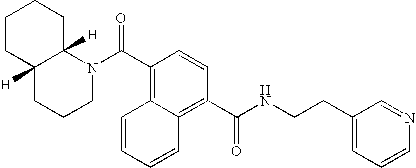 | [M + 1]⁺ 443 |
| 30-34 | 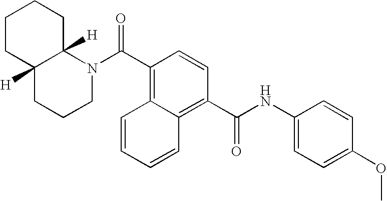 | [M + 1]⁺ 444 |

### EXAMPLE 31

The following compounds are prepared using procedures described in the previous examples.

| | | MS |
|---|---|---|
| Compd | Structure | [m/z] |
| 31-1 | 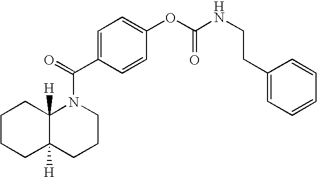 | (mp 153-154° C.) |
| 31-2 | 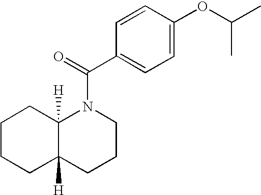 | [M + 1]⁺302 |
| 31-3 | 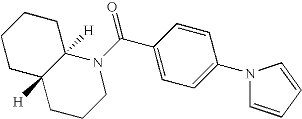 | [M + 1]⁺309 |
| 31-4 | 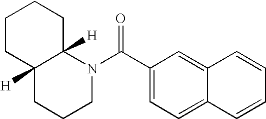 | [M + 1]⁺294 |
| 31-5 | 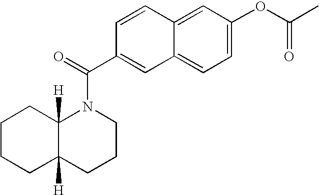 | [M + 1]⁺352 |
| 31-6 | 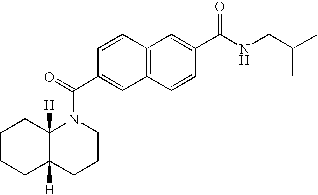 | [M + 1]⁺393 |
| 31-7 | 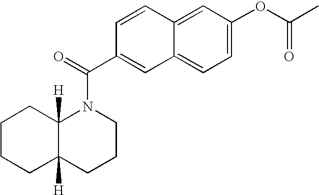 | [M + 1]⁺352 |
| 31-8 | 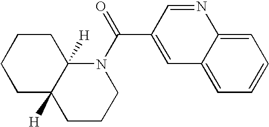 | [M + 1]⁺295 |

### EXAMPLE 32

### (4-Fluoro-phenyl)-{5-[(4aS*,8aR*)-octahydro-1(2H)-quinoline-1-carbonyl]-2,3-dihydro-indol-1-yl}-methanone

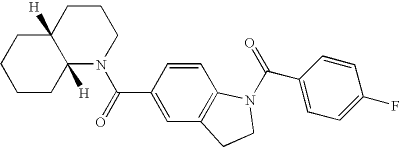

### A. 5-Bromo-2,3-dihydro-1H-indole

A solution of 1-acetyl-5-bromo-indoline (20.00 g, 83.3 mmol) and potassium hydroxide (23.33 g, 416.66 mmol) in 200 mL THF and 40 mL MeOH is refluxed for 2 h. The solution is cooled and evaporated to near dryness. The residue is taken up in water and extracted three times with diethyl ether. The diethyl ether layers are combined, washed with brine, dried over anhydrous MgSO₄, and concentrated. The product is dried under vacuum to afford 5-bromo-2,3-dihydro-1H-indole: NMR (CDCl₃) 3.0 (t, 2H), 3.6 (t, 2H), 3.75 (br s, 1H), 6.5 (d, 1 h), 7.05 (dd, 1H), 7.2 (s, 1H).

### B. 5-Bromo-2,3-dihydro-indole-1-carboxylic acid tert-butyl ester

A solution of the title A compound, 5-bromo-2,3-dihydro-1H-indole (15.75 g, 79.54 mmol) in 200 mL acetonitrile and 200 mL dichloromethane is treated with DMAP (0.971 g, 7.95 mmol) and di-t-butyl dicarbonate (19.14 g, 87.49 mmol). The solution is stirred at RT for 16 h. The mixture is diluted with 300 mL dichloromethane and washed twice with 1 N aqueous HCl and once with brine, dried over anhydrous MgSO₄, and concentrated to afford 5-bromo-2,3-dihydro-indole-1-carboxylic acid tert-butyl ester.

### C. 2,3-Dihydro-indole-1,5-dicarboxylic acid 1-tert-butyl ester

A solution of the title B compound, 5-bromo-2,3-dihydro-indole-1-carboxylic acid tert-butyl ester (15.86 g, 53.22 mmol) in 500 mL THF is cooled to −73° C. and treated with n-butyllithium (1.6 M in hexanes, 53.22 mL, 85.15 mmol). After 15 min at −73° C., dry CO₂ is bubbled through the solution for 40 min. The reaction is kept at −73° C. for 1 h, warmed to 0° C. for 1 h, and then warmed to RT for 1 h. The mixture is poured into 1 N aqueous HCl and extracted twice with diethyl ether. The diethyl ether layers are combined, washed with brine, dried over anhydrous MgSO₄, and concentrated to afford of 2,3-dihydro-indole-1,5-dicarboxylic acid 1-tert-butyl ester as an white solid: NMR (DMSO-d₆) 1.51 (s, 9H), 3.10 (t, 2H, J=8.75), 3.69 (t, 2H, J=8.80), 7.73-7.79 (m, 3H), 12.62 (br s, 1H).

### D. 5-[(4aS*,8aR*)-Octahydro-1(2H)-quinoline-1-carbonyl]-2,3-dihydro-indole-1-carboxylic acid tert-butyl ester

A solution of the title C compound, 2,3-dihydro-indole-1,5-dicarboxylic acid 1-tert-butyl ester (2.63 g, 10 mmol) in 40 mL of dichloromethane and 5 mL of DMF is cooled to 0° C. and treated with oxalyl chloride (1.13 mL, 13.0 mmol). The mixture is stirred for 30 min, then NMM (2.20 mL, 20.0 mmol) and decahydroquinoline (1.81 g, 13.0 mmol) are added sequentially. The reaction is warmed to RT and stirred for 16 h. The mixture is partitioned between EtOAc and saturated aqueous NaHCO₃. The organic layer is washed with brine, dried over anydrous Na₂SO₄, and concentrated. Chromatography on silica (eluant; 1/3—EtOAc/hexane) affordes 5-[(4aS*,8aR*)-octahydro-1(2H)-quinoline-1-carbonyl]-2,3-dihydro-indole-1-carboxylic acid tert-butyl ester: NMR (DMSO-d₆) 1.00-1.71 (m, 21H), 2.07 (br d, 2H), 3.07 (t, 2H), 3.28-3.37 (m, 2H), 3.92 (t, 2H), 7.14-7.19 (m, 3H).

### E. (2,3-Dihydro-1H-indol-5-yl)-(4aS*,8aR*)-octahydro-1(2H)-quinolin-1-yl-methanone

The title D compound, 5-[(4aS*,8aR*)-octahydro-1(2H)-quinoline-1-carbonyl]-2,3-dihydro-indole-1-carboxylic acid tert-butyl ester (1.10 g, 2.86 mmol) is dissolved in 30 mL of dichloromethane and HCl(g) is bubbled through the solution for 10 min. The flask is stoppered, and the reaction is stirred at RT for 16 h. The organics are washed with saturated aqueous NaHCO₃, water and brine, dried over anhydrous Na₂SO₄, and concentrated to afford of (2,3-dihydro-1H-indol-5-yl)-(4aS*,8aR*)-octahydro-1(2H)-quinolin-1-yl-methanone as an off white solid: NMR (CDCl₃) 1.05-1.41 (m, 5H), 1.62-1.74 (m, 7H), 2.25-2.27 (m, 1H), 3.00-3.06 (m, 2H), 3.36-3.61 (m, 5H), 3.89 (br s, 1H), 6.56 (d, 1H), 7.08-7.26 (m, 2H); API-MS 285 [M+H]⁺.

### F. (4-Fluoro-phenyl)-{5-[(4aS*,8aR*)-octahydro-1(2H)-quinoline-1-carbonyl]-2,3-dihydro-indol-1-yl}-methanone

Under parallel reaction synthesis conditions, a solution of NMM (2.0 M in THF, 98 μL, 0.195 mmol) and a solution of 4-fluorobenzoyl chloride (1.0 M in THF, 195 μL, 0.195 mmol) were dispensed sequentially into a vial containing a solution of the title E compound, (2,3-dihydro-1H-indol-5-yl)-(4aS*,8aR*)-octahydro-1(2H)-quinolin-1-yl-methanone (0.23 M in DMF, 565 μL, 0.13 mmol). The vial is agitated at RT for 16 h. A solution of aqueous lithium hydroxide (1.5 N, 100 μL, 0.15 mmol) is dispensed into the vial, and the vial is agitated for 20 min. The reaction mixture is diluted with 500 μL DMF, acidified with 50 μL TFA and purified by HPLC to afford of (4-fluoro-phenyl)-{5-[(4aS*,8aR*)-octahydro-1(2H)quinoline-1-carbonyl]-2,3-dihydro-indol-1-yl}-methanone: API-MS 408 [M+H]⁺.

### EXAMPLE 33

The following compounds are prepared analogously to Example 32 by treating the title E compound in Example 32 with the appropriate N-derivatizing agent such as an activated derivative of a carboxylic acid, a sulfonyl chloride, a chloroformate or an isocyanate.

| Compd | Structure | MS [m/z] |
|---|---|---|
| 33-1 | 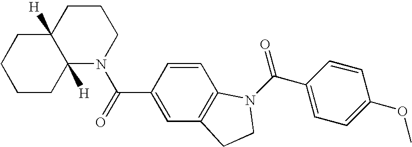 | [M + 1]⁺ 420 |
| 33-2 | 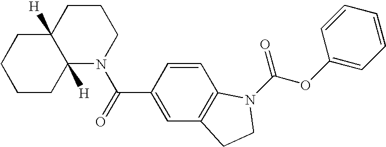 | [M + 1]⁺ 405 |
| 33-3 | 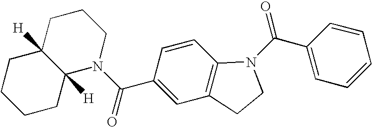 | [M + 1]⁺ 390 |
| 33-4 | 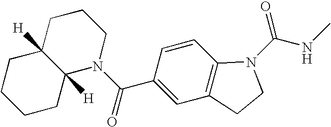 | [M + 1]⁺ 342 |
| 33-5 | 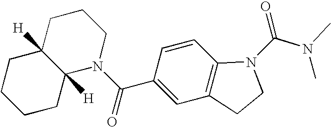 | [M + 1]⁺ 356 |
| 33-6 | 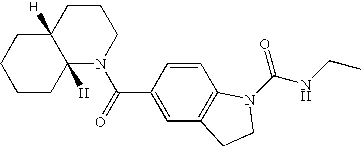 | [M + 1]⁺ 356 |
| 33-7 | 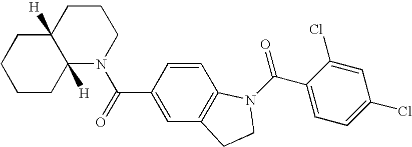 | [M + 1]⁺ 457 |
| 33-8 | 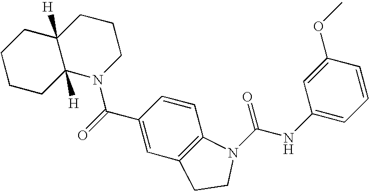 | [M + 1]⁺ 434 |
| 33-9 | 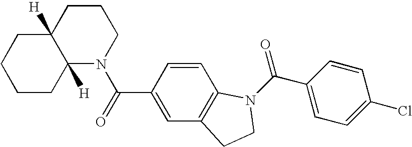 | [M + 1]⁺ 423 |
| 33-10 | 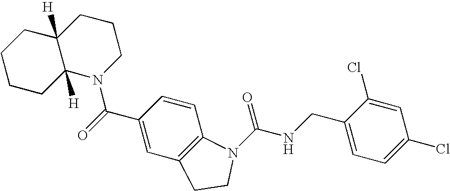 | [M + 1]⁺ 486 |
| 33-11 | 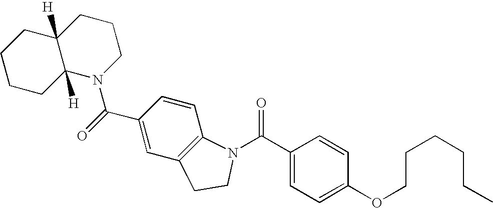 | [M + 1]⁺ 490 |
| 33-12 | 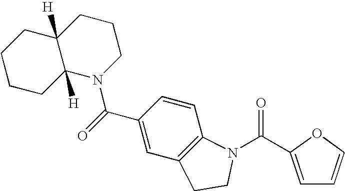 | [M + 1]⁺ 379 |
| 33-13 | 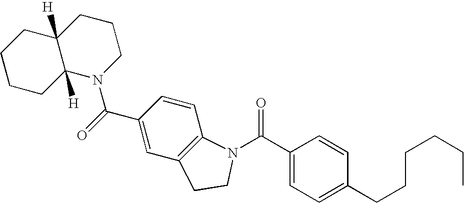 | [M + 1]⁺ 474 |
| 33-14 | 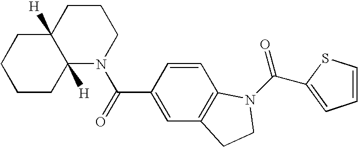 | [M + 1]⁺ 395 |
| 33-15 | 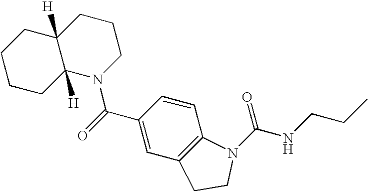 | [M + 1]⁺ 371 |
| 33-16 | 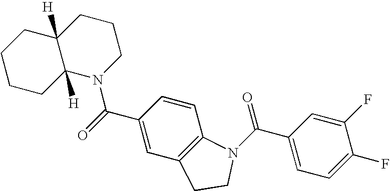 | [M + 1]⁺ 425 |
| 33-17 | 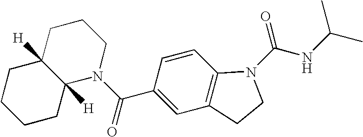 | [M + 1]⁺ 371 |
| 33-18 | 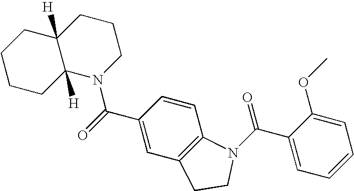 | [M + 1]⁺ 419 |
| 33-19 | 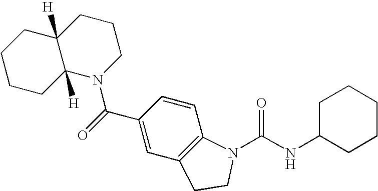 | [M + 1]⁺ 410 |
| 33-20 | 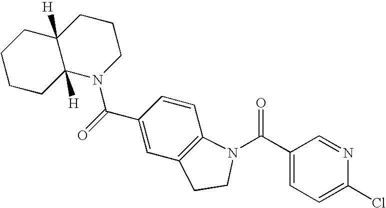 | [M + 1]⁺ 424 |
| 33-21 | 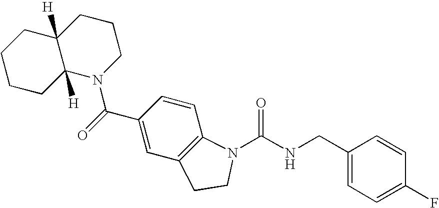 | [M + 1]⁺ 436 |
| 33-22 | 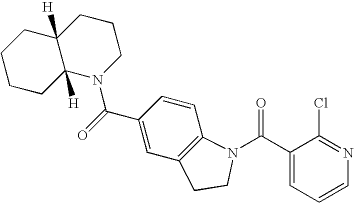 | [M + 1]⁺ 424 |
| 33-23 | 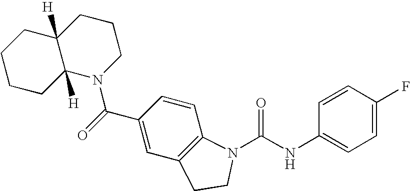 | [M + 1]⁺ 423 |
| 33-24 | 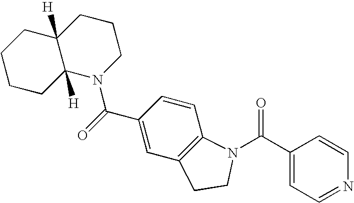 | [M + 1]⁺ 390 |
| 33-25 | 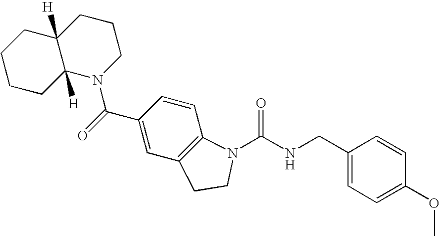 | [M + 1]⁺ 449 |
| 33-26 | 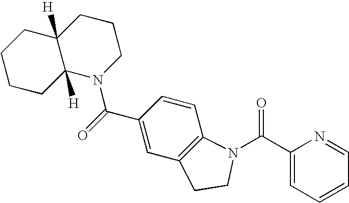 | [M + 1]⁺ 390 |
| 33-27 | 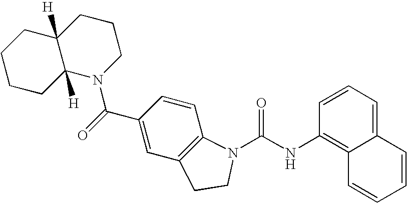 | [M + 1]⁺ 455 |
| 33-28 | 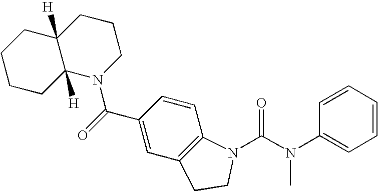 | [M + 1]⁺ 419 |
| 33-29 | 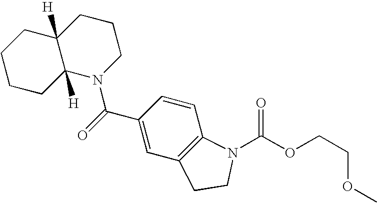 | [M + 1]⁺ 387 |
| 33-30 | 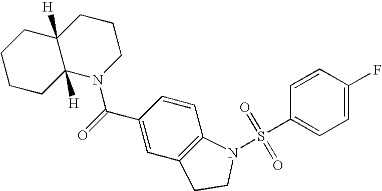 | [M + 1]⁺ 443 |
| 33-31 | 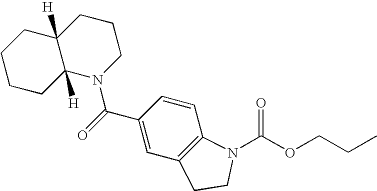 | [M + 1]⁺ 371 |
| 33-32 | 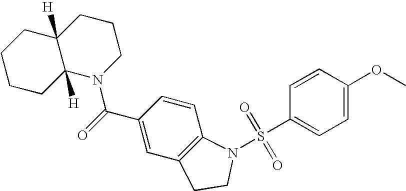 | [M + 1]⁺ 455 |
| 33-33 | 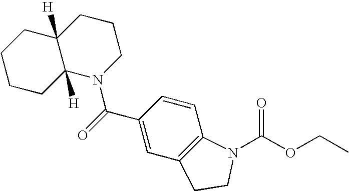 | [M + 1]⁺ 357 |
| 33-34 | 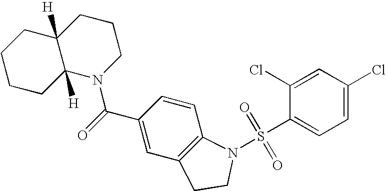 | [M + 1]⁺ 493 |
| 33-35 | 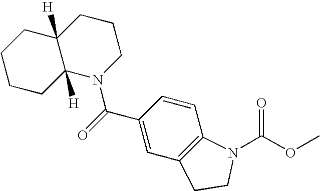 | [M + 1]⁺ 343 |
| 33-36 | 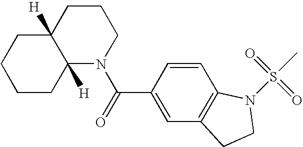 | [M + 1]⁺ 363 |
| 33-37 | 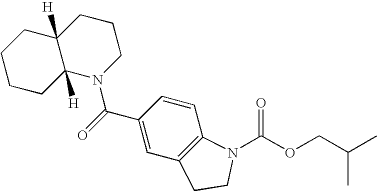 | [M + 1]⁺ 385 |
| 33-38 | 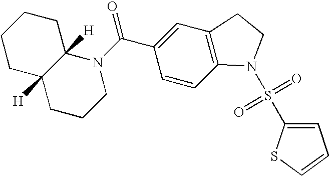 | [M + 1]⁺ 431 |
| 33-39 | 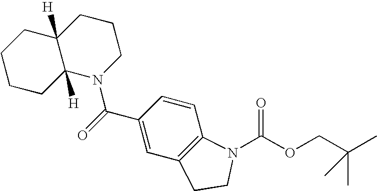 | [M + 1]⁺ 399 |
| 33-40 | 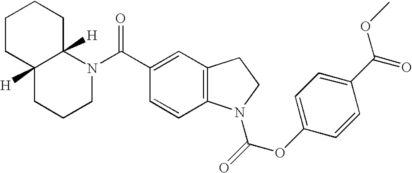 | [M + 1]⁺ 463 |

### EXAMPLE 34

### N-{3-[5-((4aS*,8aR*)-Octahydro-1(2H)-quinoline-1-carbonyl)-furan-2-yl]-phenyl}-benzamide

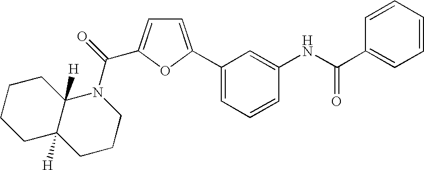

### A. [5-(3-Nitrophenyl)-furan-2-yl]-(4aS*,8aR*)-octahydro-1(2H)-quinolin-1-yl-methanone

A mixture of decahydroquinoline (2.39 g, 17.15 mmol), 5-(3-nitrophenyl)-2-furoic acid (4.0 g, 17.15 mmol), EDCI (3.29 g, 17.15 mmol) and HOAt (2.33 g, 17.15 mmol) in DMF (40 mL) is stirred at 60° C. overnight. The mixture is then partitioned between EtOAc and water. The organic phase is washed with brine, dried over anhydrous Na₂SO₄, filtered and concentrated to give the crude product. The crude product is chromatographed on silica gel using an EtOAc/hexane mixture (20:80) as the eluent to give [5-(3-nitrophenyl)-furan-2-yl]-(4aS*,8aR*)-octahydro-1(2H)-quinolin-1-yl-methanone.

### B. [5-(3-Aminophenyl)-furan 2-yl]-(4aS*,8aR*)-octahydro-1(2H)-quinolin-1-yl-methanone

A solution of the title A compound, [5-(3-nitrophenyl)-furan-2-yl]-(4aS*,8aR*)-octahydro-1(2H)-quinolin-1-yl-methanone (1.3 g, 3.67 mmol) is stirred with 130 mg of 10% Pd/C in 30 mL of EtOAc under 40 psi of hydrogen at RT overnight. The mixture is then filtered and concentrated to give [5-(3-aminophenyl)-furan-2-yl]-(4aS*,8aR*)-octahydro-1(2H)-quinolin-1-yl-methanone as a white foam.

### C. N-{3-[5-((4aS*,8aR*)-Octahydro-1(2H)-quinoline-1-carbonyl)-furan-2-yl]-phenyl}-benzamide

Benzoyl chloride (104 mg, 0.74 mmol) is added to a solution of the title B compound, [5-(3-aminophenyl)-furan-2-yl]-(4aS*,8aR*)-octahydro-1(2H)-quinolin-1-yl-methanone (200 mg, 0.61 mmol) and triethylamine (125 mg, 1.23 mmol) in dichloromethane (5 mL) at RT. The reaction is stirred overnight, concentrated, and chromatographed on silica gel using an EtOAc/hexane mixture (25/75) as the eluent to give N-{3-[5-((4aS*,8aR*)-octahydro-1(2H)-quinoline-1-carbonyl)-furan-2-yl]-phenyl}-benzamide as a white foam: m.p. 62-64° C.; API-MS 429.5 [M+1]⁺, 427.8 [M−1]⁻.

### EXAMPLE 35

The following compounds are made analogously to Example 34.

| Compd | Structure | MS [m/z] |
|---|---|---|
| 35-1 | 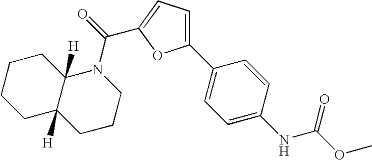 | [M + 1]⁺ 383 |
| 35-2 | 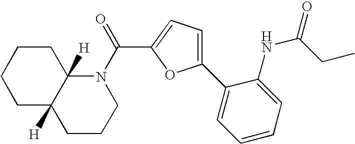 | [M + 1]⁺ 381 |
| 35-3 | 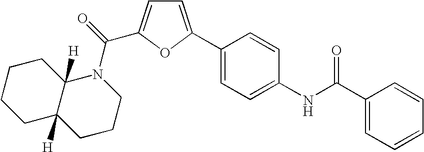 | [M + 1]⁺ 429 |
| 35-4 | 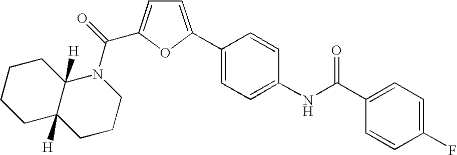 | [M + 1]⁺ 447 |
| 35-5 | 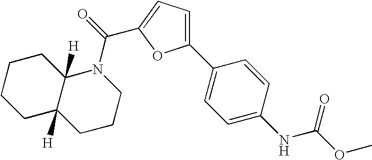 | [M + 1]⁺ 383 |
| 35-6 | 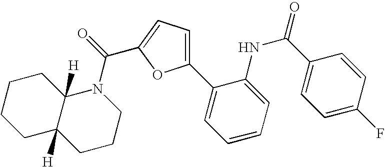 | [M + 1]⁺ 447 |
| 35-7 | 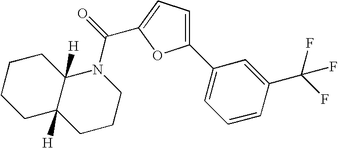 | [M + 1]⁺ 378 |
| 35-8 | 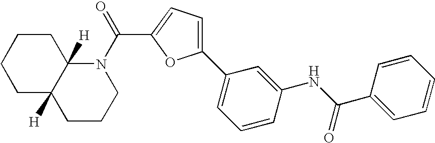 | [M + 1]⁺ 429 |
| 35-9 | 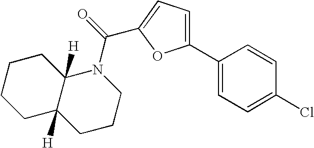 | [M + 1]⁺ 344 |
| 35-10 | 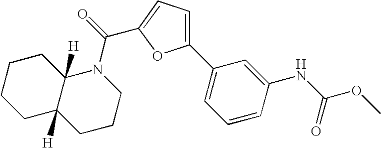 | [M + 1]⁺ 383 |
| 35-11 | 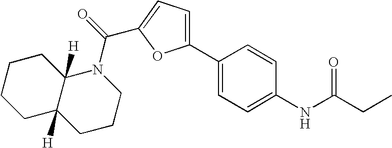 | [M + 1]⁺ 381 |
| 35-12 | 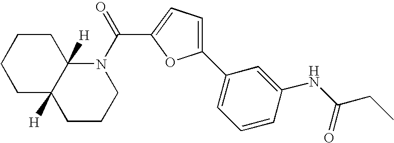 | [M + 1]⁺ 381 |
| 35-13 | 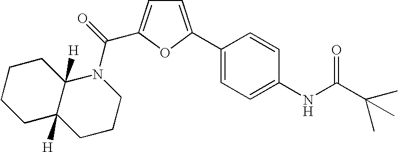 | [M + 1]⁺ 409 |
| 35-14 | 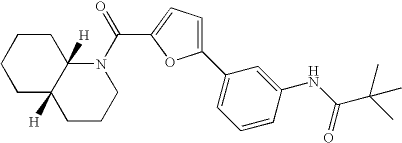 | [M + 1]⁺ 409 |
| 35-15 | 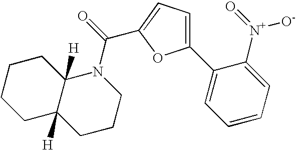 | [M + 1]⁺ 355 |
| 35-16 | 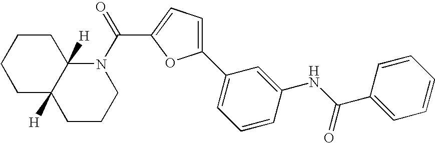 | [M + 1]⁺ 429 |
| 35-17 | 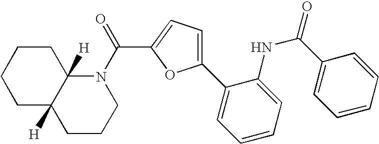 | [M + 1]⁺ 429 |
| 35-18 | 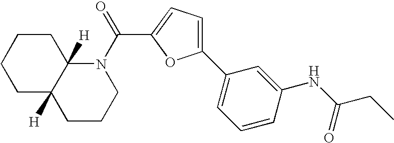 | [M + 1]⁺ 381 |
| 35-19 | 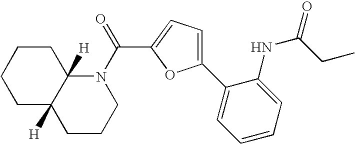 | [M + 1]⁺ 381 |
| 35-20 | 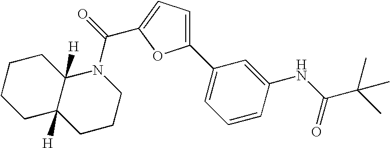 | [M + 1]⁺ 409 |
| 35-21 | 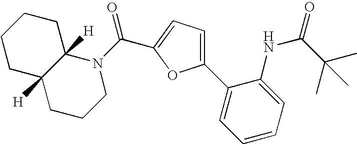 | [M + 1]⁺ 409 |
| 35-22 | 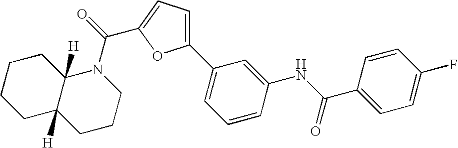 | [M + 1]⁺ 447 |
| 35-23 | 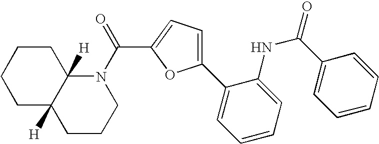 | [M + 1]⁺ 429 |
| 35-24 | 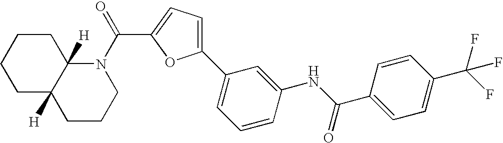 | [M + 1]⁺ 497 |

### EXAMPLE 36

### (3-Methyl-H-inden-2-yl)-(4aS*,8aR*)-octahydro-1(2H)-quinolin-1-yl-methanone

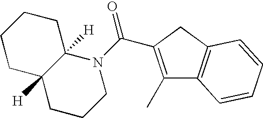

The title compound is prepared according to methods described in the previous examples: m.p. 98-100° C.; API-MS 296 [M+1]⁺.

### EXAMPLE 37

### (3-Methyl-1H-inden-2-yl)-(4aR*,8aR*)-octahydro-1(2H)-quinolin-1-yl-methanone

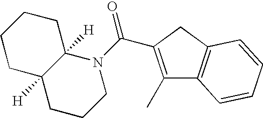

The title compound is prepared according to methods described in the previous examples: API-MS 296 [M+1]⁺.

### EXAMPLE 38

### (1-Methyl-1H-indol-2-yl)-(4aS*,8aR*)-octahydro-1(2H)-quinolin-1-yl-methanone

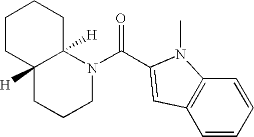

The title compound is prepared according to methods described in the previous examples: m.p. 87-90° C.; API-MS 311 [M+1]⁺.

### EXAMPLE 39

### (1-Methyl-1H-indol-2-yl)-(4aR*,8aR*)-octahydro-1(2H)-quinolin-1-yl-methanone

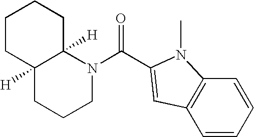

The title compound is prepared according to methods described in the previous examples: API-MS 311 [M+1]⁺.

### EXAMPLE 40

The following compounds are prepared analogously to Example 9 starting from 3-nitrobenzoyl chloride and decahydroquinoline, and treating the intermediate 3-aminobenzamide derivative analogous to the title B compound in Example 9 with the appropriate N-derivatizing agent, such as an activated derivative of a carboxylic acid, a chloroformate, an isocyanate or a thioisocyanate.

| Compd | Structure | MS [m/z] |
|---|---|---|
| 40-1 | 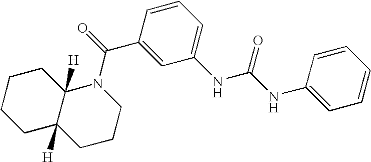 | [M + 1]⁺ 378 |
| 40-2 | 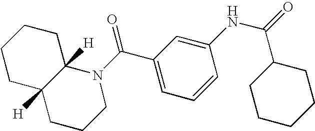 | [M + 1]⁺ 369 |
| 40-3 | 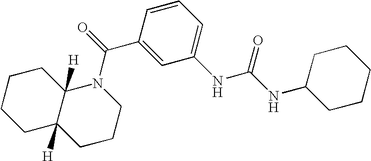 | [M + 1]⁺ 384 |
| 40-4 | 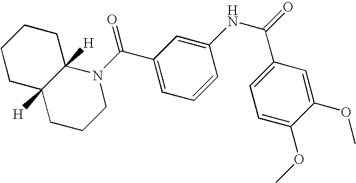 | [M + 1]⁺ 423 |
| 40-5 | 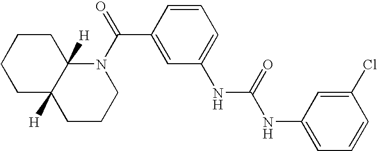 | [M + 1]⁺ 413 |
| 40-6 | 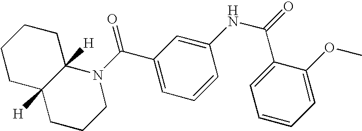 | [M + 1]⁺ 393 |
| 40-7 | 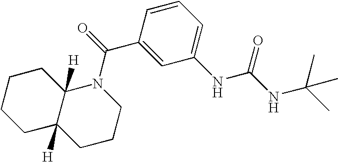 | [M + 1]⁺ 358 |
| 40-8 | 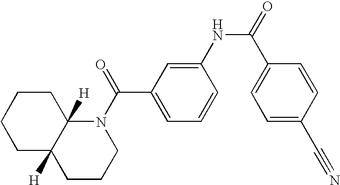 | [M + 1]⁺ 388 |
| 40-9 | 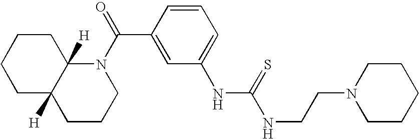 | [M + 1]⁺ 429 |
| 40-10 | 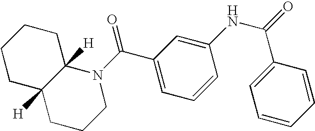 | [M + 1]⁺ 363 |
| 40-11 | 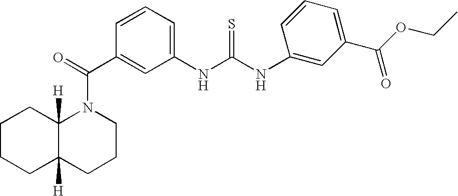 | [M + 1]⁺ 466 |
| 40-12 | 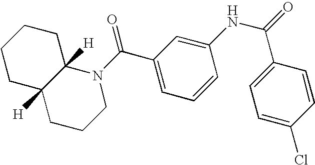 | [M + 1]⁺ 397 |
| 40-13 | 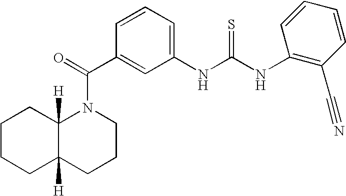 | [M + 1]⁺ 419 |
| 40-14 | 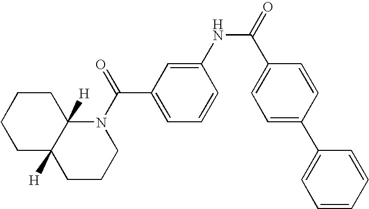 | [M + 1]⁺ 439 |
| 40-15 | 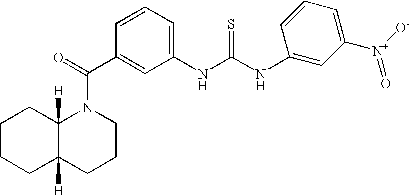 | [M + 1]⁺ 439 |
| 40-16 | 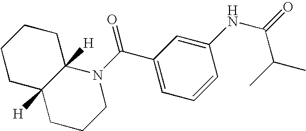 | [M + 1]⁺ 329 |

### EXAMPLE 41

### 2,4-Dichloro-N-[4-((4aS*,6S*,8aS*)-6-hydroxy-octahydro-1(2H)-quinoline-1-carbonyl)-phenyl]-benzamide

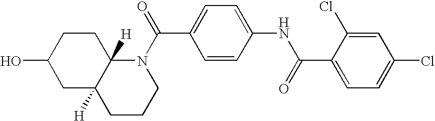

### A. (4aS*,8aS*)-Octahydro-1(2H)-quinoline-2,6-dione ethylene glycol ketal

(4aS*,8aS*)-Octahydro-quinoline-2,6-dione ethylene glycol ketal may be prepared according to methods described by Kozikowski et al., J. Org. Chem., Vol. 56, p. 4636 (1991) and Langlois et al., Bull. Soc. Chim. Fr., Vol. 130, p. 655 (1993).

### B. (4aS*,8aS*)-Octahydro-1(2H)-quinolin-6-one ethylene glycol ketal

To a suspension of 2.89 g (72 mmol) of LiAlH₄ in 50 mL of THF is slowly added 2.5 g (12 mmol) of the title A compound, (4aS*,8aS*)-octahydro-1(2H)quinoline-2,6-dione ethylene glycol ketal. The mixture is refluxed for 2 h and, after cooling to RT, 5 mL of aqueous saturated sodium carbonate (Na₂CO₃) is added cautiously. The resulting solid is filtered and washed well with dichloromethane. The filtrate is evaporated to give (4aS*,8aS*)-octahydro-1(2H)-quinolin-6-one ethylene glycol ketal as an oil; NMR (CDCl₃) 3.94 (s, 4H), 3.08 (m, 1H), 2.65 (td, 1H), 2.13 (m, 1H), 1.83-1.24 (m, 11H), 1.15-1.00 (m, 1H).

### C. (4aS*,8aS*)-1-(4-Nitro-benzoyl)-octahydro-1(2H)-quinolin-6-one ethylene glycol ketal

To a solution of 4.0 g (20 mmol) of the title B compound, (4aS*,8aS*)-octahydro-1(2H)-quinolin-6-one ethylene glycol ketal and 2.2 g (22 mmol) of triethylamine in 50 mL of dichloromethane is added dropwise a solution of 4.0 g (21 mmol) of 4-nitrobenzoyl chloride in 5 mL dichloromethane. The mixture is stirred at RT for 18 h, then water is added. The mixture is extracted with EtOAc and the organic phase is dried over anhydrous MgSO₄. The solvent is removed under reduced pressure and the residue is flash chromatographed using EtOAc/hexane (3:2) as the eluent to give (4aS*,8aS*)-1-(4-nitro-benzoyl)-octahydro-1(2H)-quinolin-6-one ethylene glycol ketal.

### D. (4aS*,8aS*)-1-(4-Amino-benzoyl)-octahydro-1(2H)-quinolin-6-one ethylene glycol ketal

A solution of 6.0 g (17 mmol) of the title C compound, 4aS*,8aS*)-1-(4-nitro-benzoyl)-octahydro-1(2H)-quinolin-6-one ethylene glycol ketal in 75 mL of EtOH is hydrogenated over 0.6 g of 10% Pd/C at 50 psi for 18 h. The catalyst is removed by filtration through Celite and the solvent is removed under reduced pressure to give (4aS*,8aS*)-1-(4-amino-benzoyl octahydro-1(2H)-quinolin-6-one ethylene glycol ketal.

### E. 2,4-Dichloro-N-[4-((4aS*,8aS*)-6-oxo-octahydro-1(2H)-quinoline-1-carbonyl)-phenyl]-benzamide ethylene glycol ketal

To a solution of 2.7 g (8.5 mmol) of the title D compound, (4aS*,8aS*)-1-(4-amino-benzoyl)-octahydro-1(2H)-quinolin-6-one ethyleneglycol ketal and 1.0 g (10 mmol) of triethylamine in 40 mL of dichloromethane is added dropwise a solution of 1.8 g (8.5 mmol) of 2,4-dichlorobenzoyl chloride in 5 mL dichloromethane. The mixture is stirred at RT for 18 h, then water is added. The mixture is extracted with EtOAc and the organic phase is dried over anhydrous Na₂SO₄, the solvent is removed under reduced pressure, and the residue flash chromatographed using hexane/EtOAc (3:2) as the eluent to give 2,4-dichloro-N-[4-((4aS*,8aS*)-6-oxo-octahydro-1(2H)-quinoline-1-carbonyl)-phenyl]-benzamide ethylene glycol ketal: m.p. 211-212°; NMR (CDCl₃) 8.18 (s, broad, 1H), 7.73, d, J=8.3, 1H), 7.62 (d, J=8.3, 2H), 7.48 (d, J=1.5, 1H), 7.43-7.34 (m, 3H), 3.97 (s, 4H), 3.62-3.30 (m, 3H), 2.32 (m, 1H), 2.02 (m, 1H), 1.88-1.51 (m, 6H), 1.43 (t, 1H), 1.26 (m, 1H).

### F. 2,4-Dichloro-N-[4-((4aS*,8aS*)-6-oxo-octahydro-1(2H)-quinoline-1-carbonyl)-phenyl]-benzamide

A solution of 0.72 g (1.47 mmol) of the title E compound, 2,4-dichloro-N-[4-((4aS*,8aS*)-6-oxo-octahydro-1(2H)-quinoline-1-carbonyl)-phenyl]-benzamide ethylene glycol ketal in 8 mL of TFA/water (1:1) is heated at 38° C. for 18 h. The solvent is removed under reduced pressure and the residue is dissolved in dichloromethane. The solution is washed with aqueous NaHCO₃ and is dried over anhydrous MgSO₄. The solvent is removed under reduced pressure and the residue is crystallized from EtOAc/hexane to give 2,4-dichloro-N-[4-((4aS*,8aS*)-6-oxo-octahydro-1(2H)-quinoline-1-carbonyl)-phenyl]-benzamide: m.p. 228-229° C.; NMR (CDCl₃) 8.07 (s, broad, 1H), 7.73 (d, J=8.3, 1H), 7.68 (d, J=8.3, 2H), 7.52-7.35 (m, 4H), 4.00 (td, 1H), 3.54 (m, 1H); 3.40 (m, 1H), 2.69-2.41 (m, 4H), 2.27-2.02 (m, 2H), 1.90-1.51 (m, 5H), 1.38 (m, 1H).

### G. 2,4-Dichloro-N-[4-((4aS*,6S*,8aS*)-6-hydroxy-octahydro-1(2H)-quinoline-1-carbonyl)-phenyl]-benzamide

To a solution of 100 mg (0.22 mmol) of the title F compound, 2,4-dichloro-N-[4-((4aS*,8aS*)-6-oxo-octahydro-1(2H)-quinoline-1-carbonyl)-phenyl]-benzamide in 10 mL of THF is added 430 mg (11 mmol) of sodium borohydride. The mixture is stirred at RT for 18 h, then EtOAc is added. The mixture is washed with water and the organic phase is dried over anhydrous MgSO₄. The solvent is removed under reduced pressure to give 2,4-dichloro-N-[4-((4aS*,6S*,8aS*)-6-hydroxy-octahydro-1(2H)-quinoline-1-carbonyl)-phenyl]-benzamide: m.p. 183-184° C.; NMR (DMSO-d₆) 7.77 (d, J=2, 1H), 7.72 (d, J=8.6, 2H), 7.63 (d, J=8.3, 1H), 7.56 (dd, 1H), 7.35 (d, J=8.6, 2H), 4.56 (d, J=4.6, 1H), 3.47 (m, 1H), 3.39-3.19 (m, 4H), 2.07 (m, 1H), 1.84 (m, 2H), 1.70 (m, 1H), 1.64-1.44 (m, 4H), 1.26-1.12 (m, 2H), 1.03-0.93 (m, 1H).

### EXAMPLE 42

### 2,4-Dichloro-N-[4-(4aS*,6R*,8aS*)-6-hydroxy-octahydro-1(2H)-quinoline-1-carbonyl)-phenyl]-benzamide

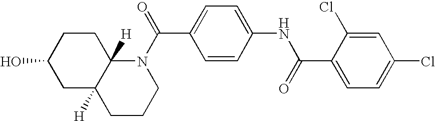

To a solution of 200 mg (0.42 mmol) of the title F compound in Example 41, 2,4-dichloro-N-[4-((4aS*,8aS*)-6-oxo-octahydro-1(2H)-quinoline-1-carbonyl)-phenyl]-benzamide in 2 mL of THF is added dropwise 0.45 mL of K-selectride (1 M in THF). The mixture is stirred for 90 min then is quenched with water. The mixture is extracted with EtOAc and the organic phase dried over magnesium sulfate. The solvent is removed under reduced pressure and the residual solid is crystallized from EtOAc/hexane to give 2,4-dichloro-N-[4-((4aS*,6R*,8aS*)-6-hydroxy-octahydro-1(2H)-quinoline-1-carbonyl)-phenyl]-benzamide: m.p. 248-250° C.; NMR (DMSO-d₆) 10.67 (s, 1H), 7.76 (d, J=2, 1H), 7.72 (d, J=8.6, 2H), 7.64 (d, J=8.3, 1H), 7.56 (dd, 1H), 7.35 (d, J=8.6, 2H), 4.41 (d, J=3, 1H), 3.86 (m, 1H), 3.40-3.24 (m, 2H), 2.11 (m, 1H), 1.86 (m, 1H), 1.79-1.39 (m, 7H), 123-1.05 (m, 2H).

### EXAMPLE 43

### 2,4-Dichloro-N-[4-((4aS*,6S*,86S*)-6-hydroxy 6-methyl-octahydro-1(2H)-quinoline-1-carbonyl)-phenyl]-benzamide

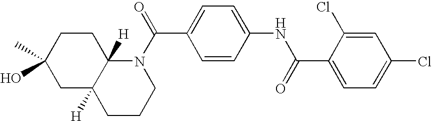

To a solution of 500 mg (1.1 mmol) of the title F compound in Example 41, 2,4-dichloro-N-[4-((4aS*,8aS*)-6-oxo-octahydro-1(2H)-quinoline-1-carbonyl)-phenyl]-benzamide in 5 mL of THF at 0° C. is added slowly 2.25 mmol of methyllithium (1.4 M in diethyl ether). After addition, the mixture is stirred at RT for 2 h. Water is added and the mixture is extracted with EtOAc. The organic phase is dried over anhydrous MgSO₄ and the solvent is removed under reduced pressure to give 2,4-dichloro-N-[4-((4aS*,6S*,8aS*)-6-hydroxy-6-methyl-octahydro-1(2H)-quinoline-1-carbonyl)-phenyl]-benzamide as a solid: m.p. 233-234° C.; NMR (DMSO-d₆) 10.68 (s, 1H), 7.76 (d, J=2, 1H), 7.72 (d, J=8.6, 2H), 7.64 (d, J=8.3, 1H), 7.55 (dd, 1H), 7.36 (d, J=8.6, 2H), 4.34 (s, 1H), 3.43 (m, 1H), 3.35-3.16 (m, 2H), 1.99 (m, 1H), 1.83-1.72 (m, 1H), 1.69-1.46 (m, 6H), 1.42 (m, 1H), 1.31 1.04 (m, 3H), 1.18 (s, 3H).

### EXAMPLE 44

### 2,4-Dichloro-N-[4-((4aS*,8aS*)-6,6-dimethyl-octahydro-1(2H)-quinoline-1-carbonyl)-phenyl]-benzamide

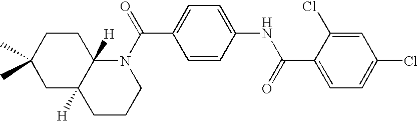

### A. 1-(4,4-Dimethyl-cyclohex-1-enyl)-pyrrolidine

To a solution of 9.36 g (74 mmol) of 4,4-dimethylcyclohexanone in 300 mL toluene is added 12 g (169 mmol) of pyrrolidine followed by 0.6 g of p-toluenesulfonic acid. The mixture is refluxed for 5 h, then the solvent is removed under reduced pressure to give 1-(4,4-dimethyl-cyclohex-1-enyl)-pyrrolidine as a brown oil.

### B. (4aS*,8aS*)-6,6-Dimethyl-octahydro-1(2H)-quinolin-2-one

A solution of the title A compound, 1-(4,4-dimethyl-cyclohex-1-enyl)-pyrrolidine and 14 g of acrylamide in 300 mL of dioxane is refluxed for 18 h. Water is added and the mixture is extracted with dichloromethane. The organic phase is dried over anhydrous MgSO₄ and the solvent is removed under reduced pressure. The residue is flash chromatographed using 10% EtOAc in hexane as the eluent to give mixture of 6,6-dimethyl-3,4,5,6,7,8-hexahydro-1H-quinolin-2-one and 6,6-dimethyl-3,4,4a,5,6,7-hexahydro-1H-quinolin-2-one. To a solution of the previous material (7.0 g, 39 mmol) in 150 mL of acetic acid is added 25 g (400 mmol) of sodium cyanoborohydride and the mixture is stirred at RT for 18 h. Saturated aqueous Na₂CO₃ is carefully added and the mixture is extracted with dichloromethane. The organic phase is dried over anhydrous MgSO₄ and the solvent is removed under reduced pressure to give (4aS*,8aS*)-6,6-dimethyl-octahydro-1(2H)-quinolin-2-one: NMR (CDCl₃) 6.80 (s, broad, 1H), 2.82 (m, 1H), 2.42 (m, 2H), 1.67 (m, 2H), 1.57-1.14 (m, 7H), 0.96 (s, 3H), 0.95 (s, 3H).

### C. (4aS*,8aS*)-6,6-Dimethyl-decahydro-quinoline

To a solution of 8.6 g (228 mmol) of LiAlH₄ in 150 mL of THF is added a solution of 7.0 g (38 mmol) of the title B compound, (4aS*,8aS*)-6,6-dimethyl-octahydro-1(2H)-quinolin-2-one in 50 mL of THF. The mixture is refluxed for 4 h, then quenched carefully with saturated aqueous NaOH solution. The mixture is extracted with diethyl ether and the organic phase is dried over anhydrous MgSO₄. The solvent is removed under reduced pressure to give (4aS*,8aS*)-6,6-dimethyl-decahydro-quinoline as an oil: NMR (CDCl₃) 3.03 (m, 1H), 2.63 (m, 1H), 2.05-1.80 (m, 2H), 1.69-1.11 (m, 9H), 1.04-0.87 (m, 2H), 0.93 (s, 3H), 0.90 (s, 3H).

### D. [(4aS*,8aS*)-6,6-Dimethyl-octahydro-1(2H)-quinolin-1-yl]-(4-nitro-phenyl)-methanone

To a solution of 2.5 g (15 mmol) of the title C compound, (4aS*,8aS*)-6,6-dimethyl-decahydro-quinoline and 1.7 g (17 mmol) of triethylamine in 50 mL of dichloromethane is added dropwise a solution of 2.8 g (15 mmol) of 4-nitrobenzoyl chloride in 10 mL of dichloromethane. The mixture is stirred at RT for 18 h, then water is added. The mixture is extracted with EtOAc and the organic phase is dried over anhydrous MgSO₄. The solvent is removed under reduced pressure and the residue is crystallized from EtOAc hexane to give [(4aS*,8aS*)-6,6-dimethyl-octahydro-1(2H)-quinolin-1-yl]-(4-nitro-phenyl)-methanone: m.p. 4-125° C.; NMR (CDCl₃) 8.26 (d, J=8.3, 2H), 7.55 (d, J=8.7, 2H), 3.48-3.20 (m, 3H), 2.12 (m, 1H), 1.90 (m, 1H), 1.73-1.59 (m, 4H), 1.53-1.32 (m, 3H), 1.20 (m, 11H), 1.04 (t, 1H), 1.00 (s, 3H), 0.94 (s, 3H).

### E. (4-Amino-phenyl)-((4aS*,8aS*)-6,6-dimethyl-octahydro-quinolin-1-yl)-methanone

A solution of 2.2 g (7 mmol) of the title D compound, [(4aS*,8aS*)-6,6-dimethyl-octahydro-1(2H)-quinolin-1-yl]-(4-nitro-phenyl)-methanone in 30 mL of EtOH is hydrogenated over 0.22 g of 10% Pd/C at 50 psi for 18 h. The catalyst is removed by filtration through Celite and the solvent is concentrated under reduced pressure to give (4-amino-phenyl)-((4aS*,8aS*)-6,6-dimethyl-octahydro-quinolin-1-yl)-methanone as a thick oil; NMR (CDCl₃) 7.27 (d, J=8.7, 2H), 6.64 (d, J=8.3, 2H), 3.83 (s, broad, 2H), 3.47 (m, 2H), 3.32 (dt, 1H), 2.10 (m, 1H), 1.87 (m, 1H), 1.76-1.52 (m, 5H), 1.47-1.32 (m, 3H), 1.18-1.03 (m, 1H), 0.99 (s, 3H), 0.92 (s, 3H).

### F. 2,4-Dichloro-N-[4-((4aS*,8aS*)-6,6-dimethyl-octahydro-1(2H)-quinoline-1-carbonyl)-phenyl]-benzamide

To a solution of 0.5 g (1.7 mmol) of the title E compound, (4-amino-phenyl)-((4aS*,8aS*)-6,6-dimethyl-octahydro-quinolin-1-yl)-methanone and 0.2 g (2 mmol) of triethylamine in 10 mL of dichloromethane is added dropwise a solution of 0.37 g (1.8 mmol) of 2,4-dichlorobenzoyl chloride in 2 mL of dichloromethane. The mixture is stirred at RT for 18 h, then water is added. The mixture is extracted with EtOAc and the organic phase is dried over anhydrous MgSO₄. The solvent is removed under reduced pressure and the residue crystallized from EtOAc/hexane to give 2,4-dichloro-N-[4-((4aS*,8aS*)-6,6-dimethyl-octahydro-1(2H)-quinoline-1-carbonyl)-phenyl]-benzamide: m.p. 220-221° C.; NMR (CDCl₃) 8.22 (s, broad, 1H), 7.73 (d, J=8.3, 1H), 7.60 (d, J=8.3, 2H), 7.48 (d, J=2, 1H), 7.43-7.34 (m, 3H), 3.41 (m, 2H), 3.32 (dt, 1H), 2.08 (m, 1H), 1.87 (m, 1H), 1.74-1.50 (m, 4H), 1.50-1.29 (m, 3H), 1.16 (m, 1H), 1.02 (t, 1H), 0.99 (s, 3H), 0.94 (s, 3H).

### EXAMPLE 45

### 2,4-Dichloro-N-[4-((4aS*,8aS*)-octahydro-1,4-benzoxazine-4-carbonyl)-phenyl]-benzamide

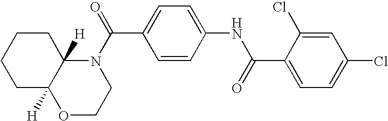

The title compound is prepared analogously to the previous examples starting from (4aS*,8aS*)-octahydro-1,4-benzoxazine (prepared according to methods described by Bettoni et al., Tetrahedron, Vol. 36, p. 409 (1980)) and 4-nitrobenzoyl chloride: m.p. 227-230° C.; NMR (DMSO-d₆) 10.74 (s, 1H), 7.80-7.75 (m, 3H), 7.65 (d, J=8.3, 1H), 7.57 (dd, 1H), 7.45 (d, J=8.3, 2H), 3.84-3.68 (m, 3H), 3.62 (m, 1H), 3.49-3.32 (m, 2H), 2.33 (d, broad, J=13, 1H), 1.88 (m, 1H), 1.75-1.68 (m, 2H), 1.48-1.15 (m, 4H).

### EXAMPLE 46

The following compounds are prepared analogously to Example 9 starting from 4-nitrobenzoyl chloride and either trans or cis decahydroisoquinoline, and treating the intermediate 4-aminobenzamide derivative with the appropriate N-derivatizing agent such as an activated derivative of a carboxylic acid, a chloroformate or an isocyanate.

| Compd | Structure | MS [m/z] |
|---|---|---|
| 46-1 | 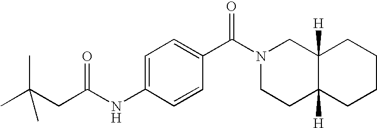 | [M + 1]⁺ 358 |
| 46-2 | 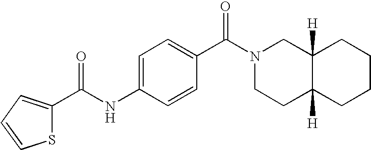 | [M + 1]⁺ 369 |
| 46-3 | 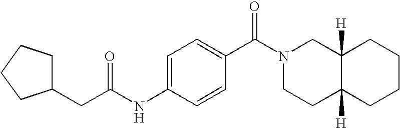 | [M + 1]⁺ 370 |
| 46-4 | 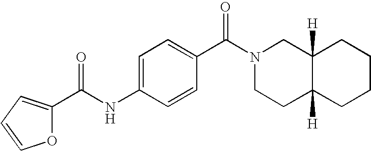 | [M + 1]⁺ 353 |
| 46-5 | 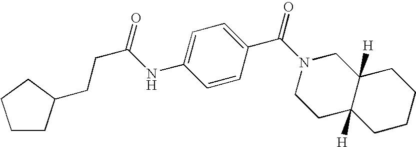 | [M + 1]⁺ 384 |
| 46-6 | 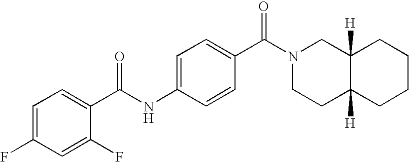 | [M + 1]⁺ 399 |
| 46-7 | 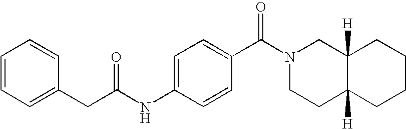 | [M + 1]⁺ 378 |
| 46-8 | 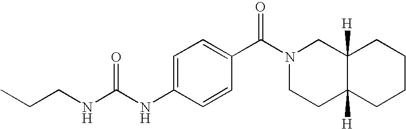 | [M + 1]⁺ 344 |
| 46-9 | 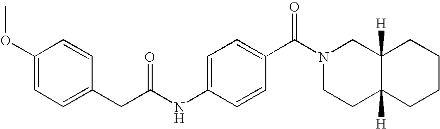 | [M + 1]⁺ 408 |
| 46-10 | 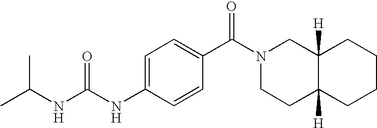 | [M + 1]⁺ 344 |
| 46-11 | 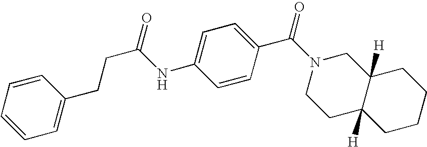 | [M + 1]⁺ 392 |
| 46-12 | 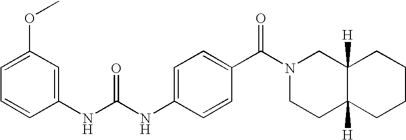 | [M + 1]⁺ 408 |
| 46-13 | 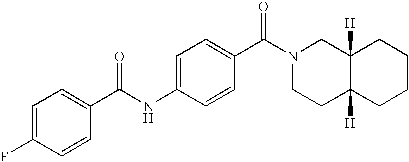 | [M + 1]⁺ 381 |
| 46-14 | 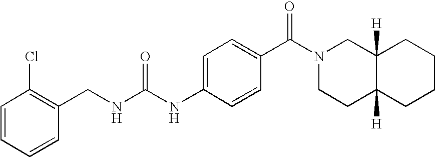 | [M + 1]⁺ 426 |
| 46-15 | 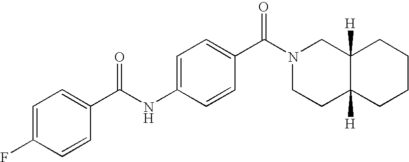 | [M + 1]⁺ 381 |
| 46-16 | 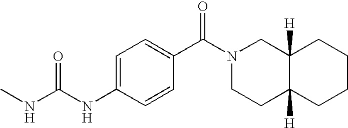 | [M + 1]⁺ 316 |
| 46-17 | 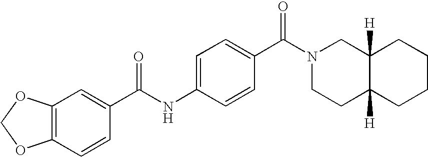 | [M + 1]⁺ 407 |
| 46-18 | 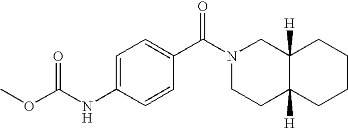 | [M + 1]⁺ 317 |
| 46-19 | 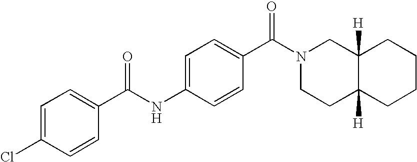 | [M + 1]⁺ 397 |
| 46-20 | 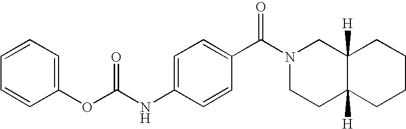 | [M + 1]⁺ 379 |
| 46-21 | 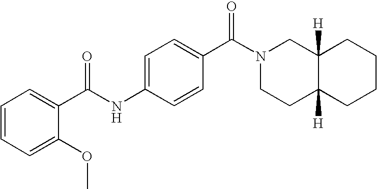 | [M + 1]⁺ 393 |
| 46-22 | 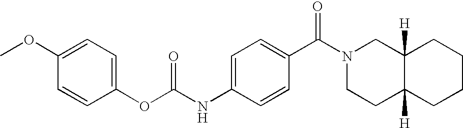 | [M + 1]⁺ 409 |
| 46-23 | 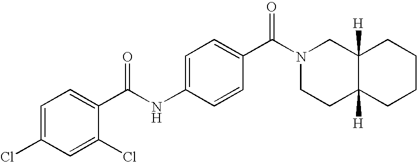 | [M + 1]⁺ 431 |
| 46-24 | 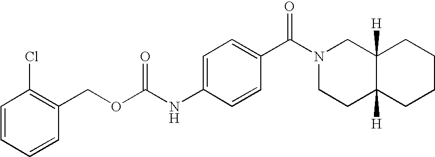 | [M + 1]⁺ 427 |

### EXAMPLE 47

The following compounds are prepared analogously to Example 9 starting from 2-chloro-4-nitrobenzoyl chloride and either trans or cis decahydroisoquinoline, and treating the intermediate 4-amino-2-chlorobenzamide derivative with the appropriate N-derivatizing agent such as an activated derivative of a carboxylic acid, a chloroformate or an isocyanate.

| Compd | Structure | MS [m/z] |
|---|---|---|
| 47-1 | 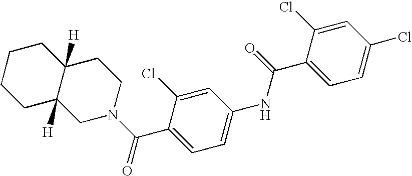 | [M + 1]⁺ 465 |
| 47-2 | 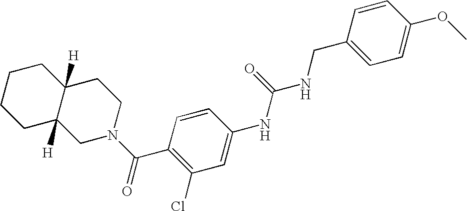 | [M + 1]⁺ 456 |
| 47-3 | 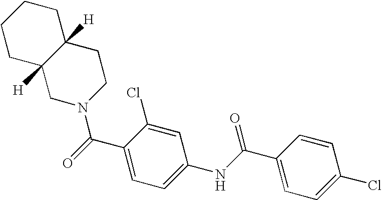 | [M + 1]⁺ 431 |
| 47-4 | 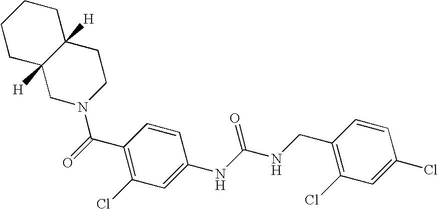 | [M + 1]⁺ 496 |
| 47-5 | 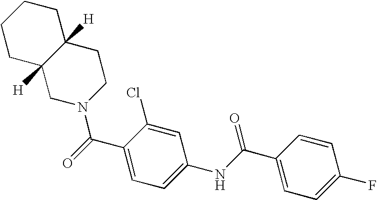 | [M + 1]⁺ 415 |
| 47-6 | 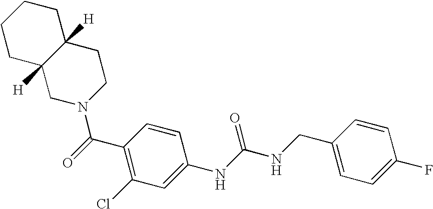 | [M + 1]⁺ 444 |
| 47-7 | 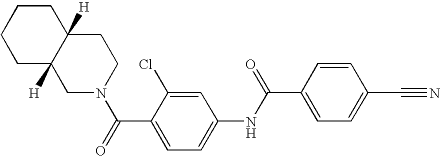 | [M + 1]⁺ 422 |
| 47-8 | 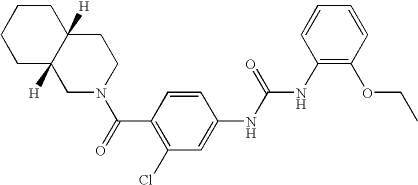 | [M + 1]⁺ 456 |
| 47-9 | 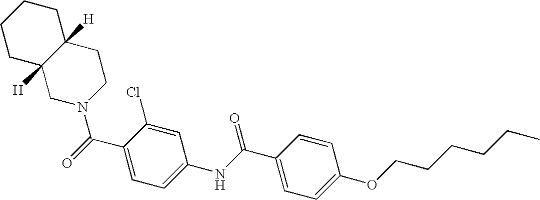 | [M + 1]⁺ 498 |
| 47-10 | 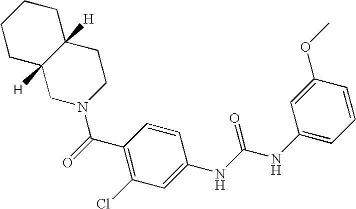 | [M + 1]⁺ 442 |
| 47-11 | 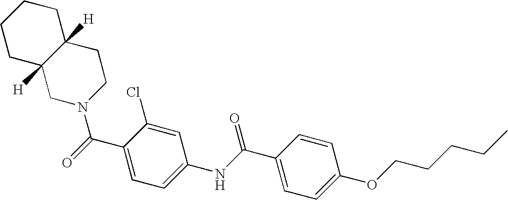 | [M + 1]⁺ 483 |
| 47-12 | 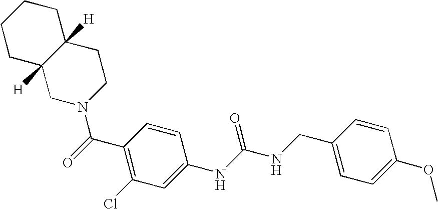 | [M + 1]⁺ 456 |
| 47-13 | 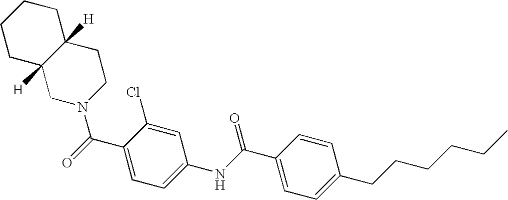 | [M + 1]⁺ 482 |
| 47-14 | 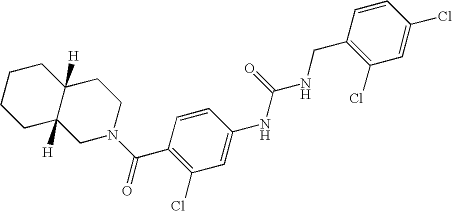 | [M + 1]⁺ 496 |
| 47-15 | 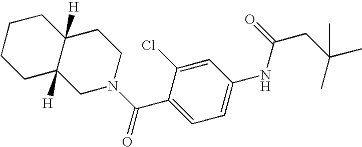 | [M + 1]⁺ 391 |
| 47-16 | 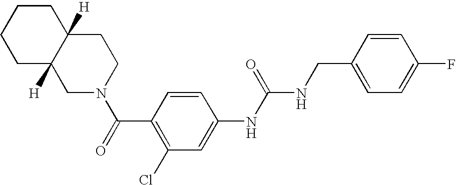 | [M + 1]⁺ 444 |
| 47-17 | 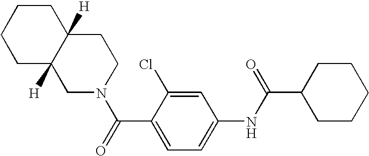 | [M + 1]⁺ 403 |
| 47-18 | 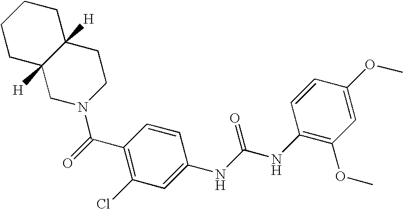 | [M + 1]⁺ 473 |
| 47-19 | 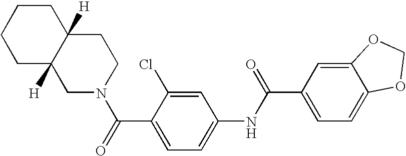 | [M + 1]⁺ 441 |
| 47-20 | 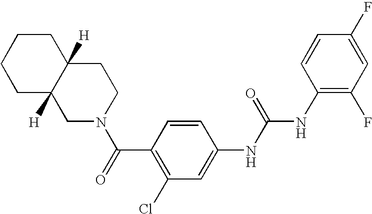 | [M + 1]⁺ 449 |
| 47-21 | 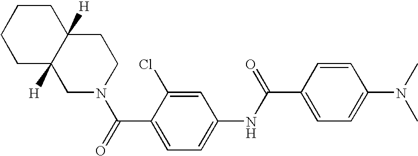 | [M + 1]⁺ 440 |
| 47-22 | 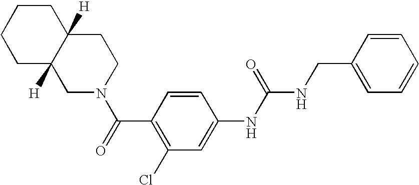 | [M + 1]⁺ 427 |
| 47-23 | 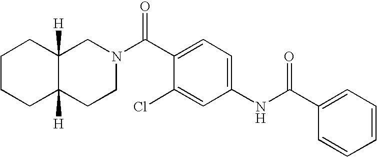 | [M + 1]⁺ 397 |
| 47-24 | 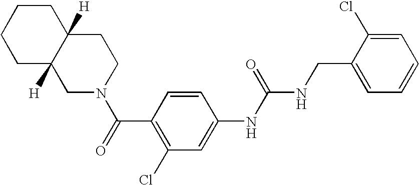 | [M + 1]⁺ 460 |
| 47-25 | 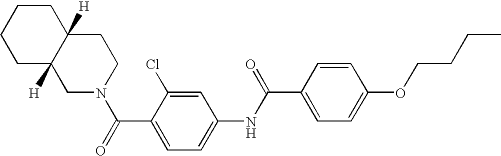 | [M + 1]⁺ 469 |
| 47-26 | 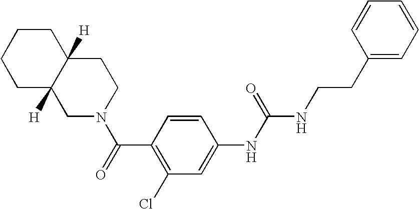 | [M + 1]⁺ 441 |
| 47-27 | 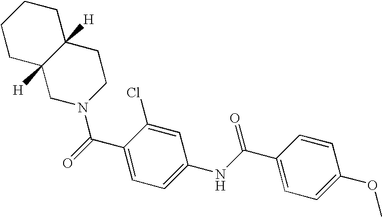 | [M + 1]⁺ 427 |
| 47-28 | 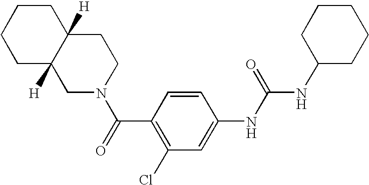 | [M + 1]⁺ 419 |
| 47-29 | 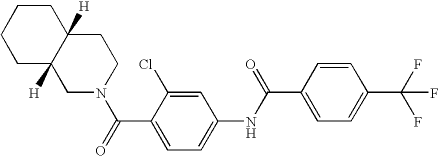 | [M + 1]⁺ 465 |
| 47-30 | 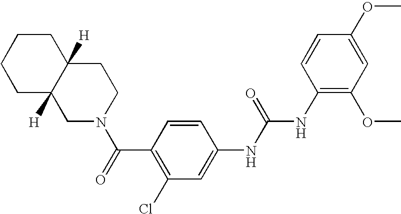 | [M + 1]⁺ 473 |
| 47-31 | 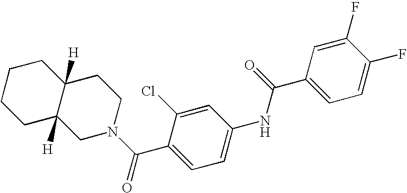 | [M + 1]⁺ 433 |
| 47-32 | 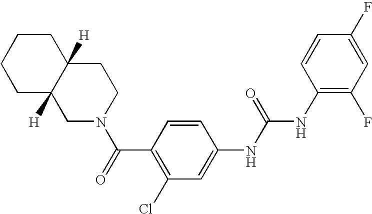 | [M + 1]⁺ 448 |
| 47-33 | 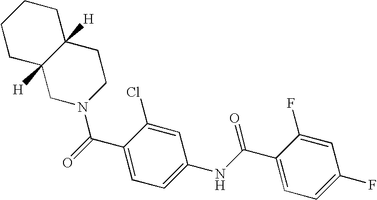 | [M + 1]⁺ 433 |
| 47-34 | 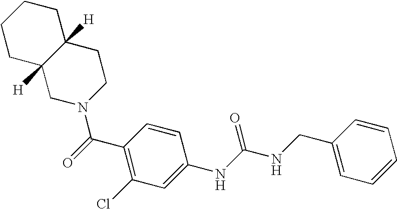 | [M + 1]⁺ 427 |
| 47-35 | 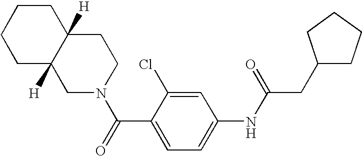 | [M + 1]⁺ 403 |
| 47-36 | 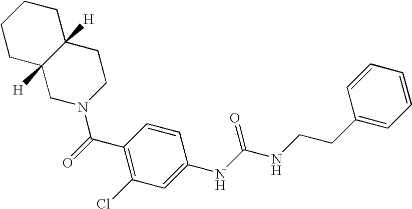 | [M + 1]⁺ 441 |
| 47-37 | 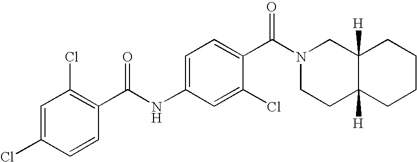 | [M + 1]⁺ 465 |
| 47-38 | 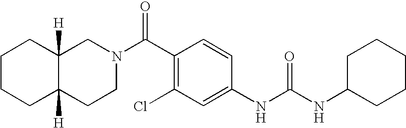 | [M + 1]⁺ 419 |
| 47-39 | 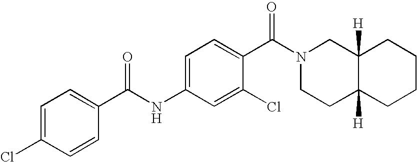 | [M + 1]⁺ 431 |
| 47-40 | 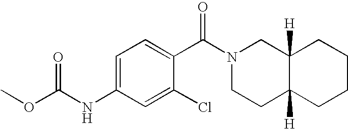 | [M + 1]⁺ 351 |
| 47-41 | 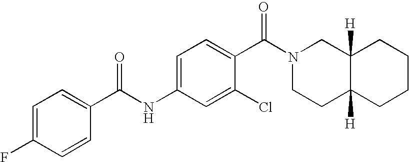 | [M + 1]⁺ 415 |
| 47-42 | 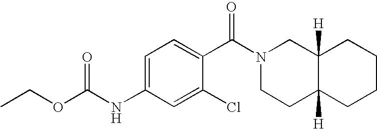 | [M + 1]⁺ 365 |
| 47-43 | 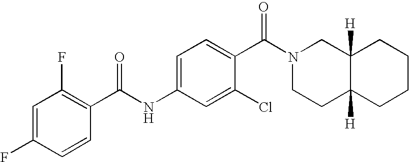 | [M + 1]⁺ 433 |
| 47-44 | 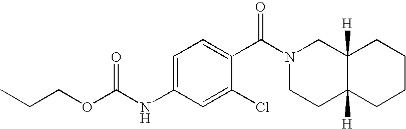 | [M + 1]⁺ 379 |
| 47-45 | 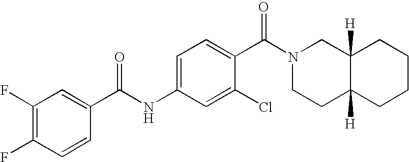 | [M + 1]⁺ 433 |
| 47-46 | 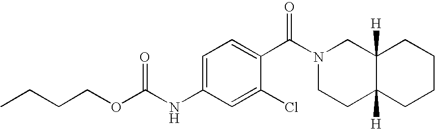 | [M + 1]⁺ 393 |
| 47-47 | 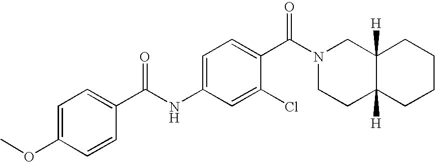 | [M + 1]⁺ 427 |
| 47-48 | 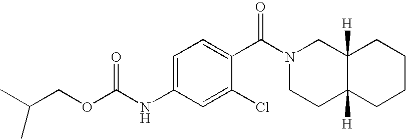 | [M + 1]⁺ 393 |
| 47-49 | 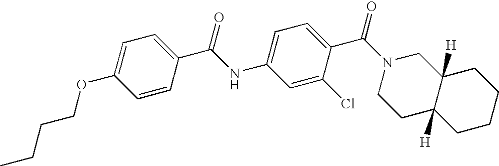 | [M + 1]⁺ 469 |
| 47-50 | 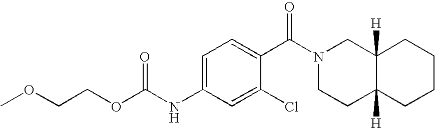 | [M + 1]⁺ 395 |
| 47-51 | 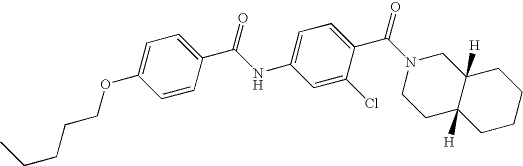 | [M + 1]⁺ 483 |
| 47-52 | 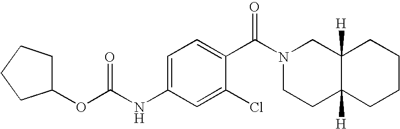 | [M + 1]⁺ 405 |
| 47-53 | 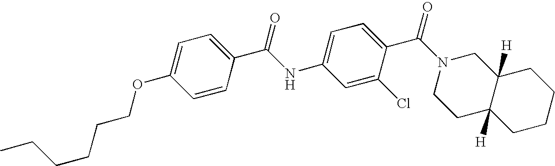 | [M + 1]⁺ 497 |
| 47-54 | 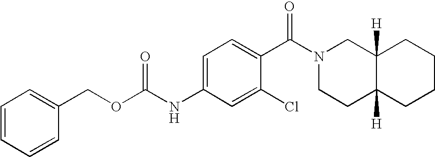 | [M + 1]⁺ 427 |
| 47-55 | 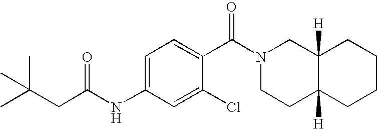 | [M + 1]⁺ 391 |
| 47-56 | 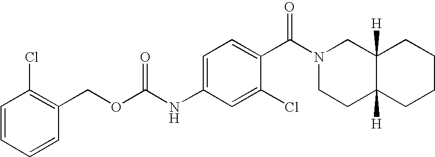 | [M + 1]⁺ 461 |
| 47-57 | 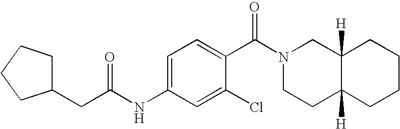 | [M + 1]⁺ 403 |
| 47-58 | 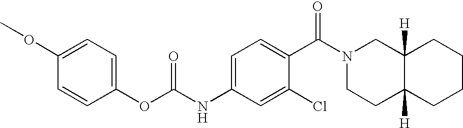 | [M + 1]⁺ 443 |
| 47-59 | 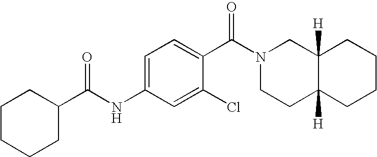 | [M + 1]⁺ 403 |
| 47-60 | 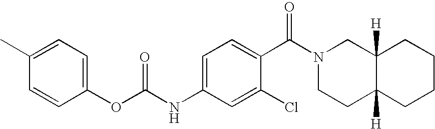 | [M + 1]⁺ 427 |
| 47-61 | 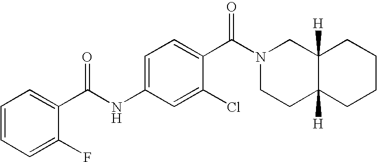 | [M + 1]⁺ 415 |
| 47-62 | 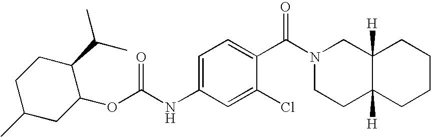 | [M + 1]⁺ 476 |
| 47-63 | 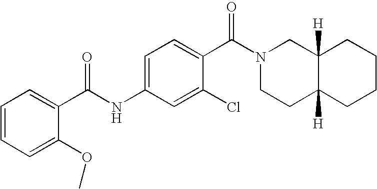 | [M + 1]⁺ 427 |
| 47-64 | 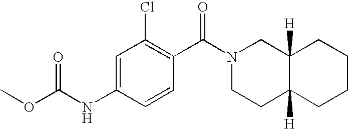 | [M + 1]⁺ 351 |
| 47-65 | 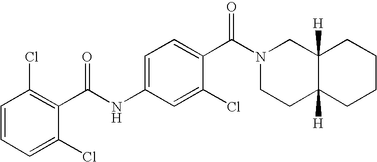 | [M + 1]⁺ 465 |
| 47-66 | 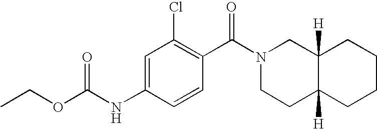 | [M + 1]⁺ 365 |
| 47-67 | 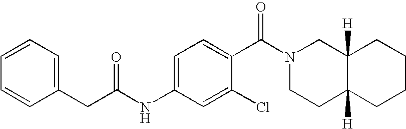 | [M + 1]⁺ 411 |
| 47-68 | 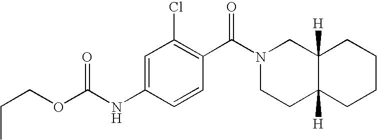 | [M + 1]⁺ 379 |
| 47-69 | 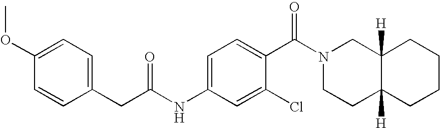 | [M + 1]⁺ 441 |
| 47-70 | 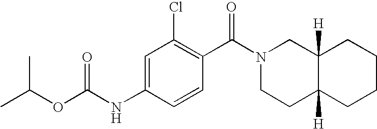 | [M + 1]⁺ 379 |
| 47-71 | 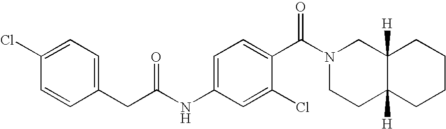 | [M + 1]⁺ 445 |
| 47-72 | 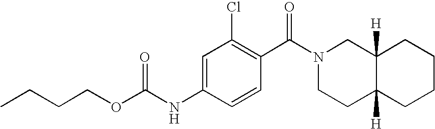 | [M + 1]⁺ 393 |
| 47-73 | 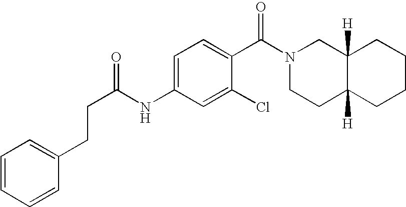 | [M + 1]⁺ 425 |
| 47-74 | 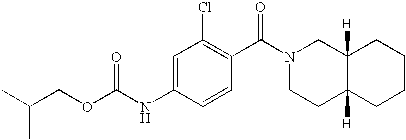 | [M + 1]⁺ 393 |
| 47-75 | 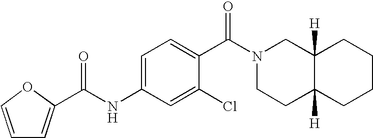 | [M + 1]⁺ 387 |
| 47-76 | 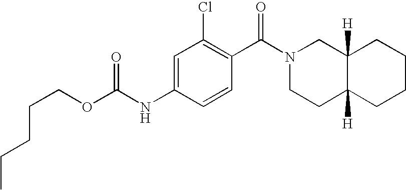 | [M + 1]⁺ 407 |
| 47-77 | 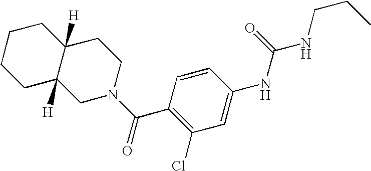 | [M + 1]⁺ 378 |
| 47-78 | 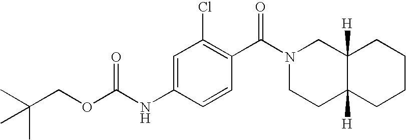 | [M + 1]⁺ 407 |
| 47-79 | 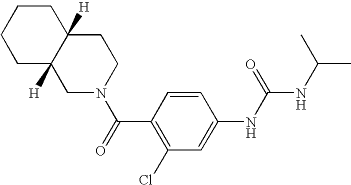 | [M + 1]⁺ 378 |
| 47-80 | 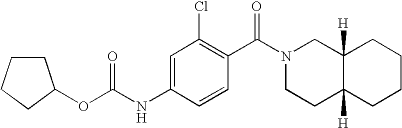 | [M + 1]⁺ 405 |
| 47-81 | 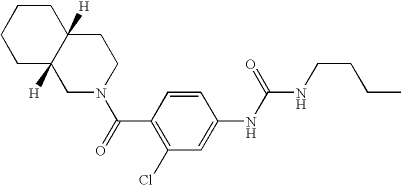 | [M + 1]⁺ 392 |
| 47-82 | 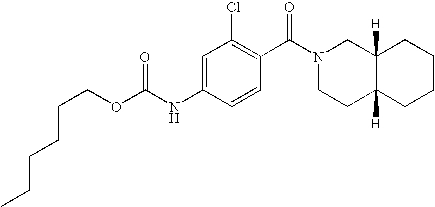 | [M + 1]⁺ 422 |
| 47-83 | 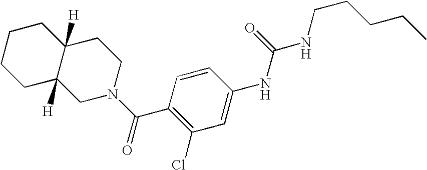 | [M + 1]⁺ 406 |
| 47-84 | 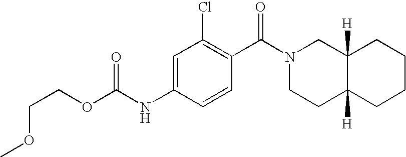 | [M + 1]⁺ 395 |
| 47-85 | 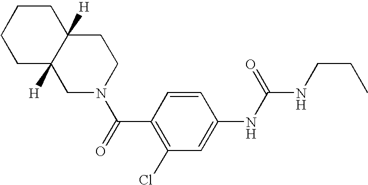 | [M + 1]⁺ 378 |
| 47-86 | 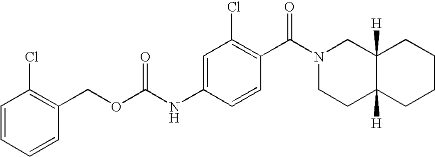 | [M + 1]⁺ 461 |
| 47-87 | 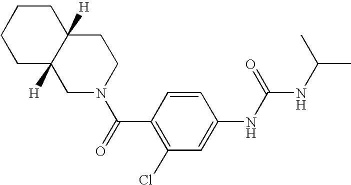 | [M + 1]⁺ 378 |
| 47-88 | 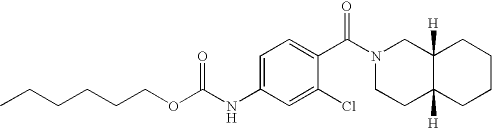 | [M + 1]⁺ 421 |
| 47-89 | 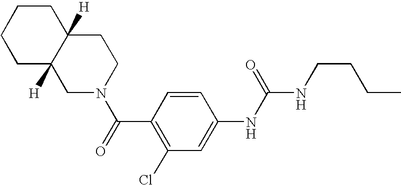 | [M + 1]⁺ 392 |
| 47-90 | 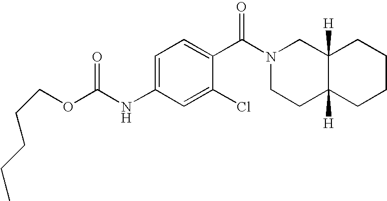 | [M + 1]⁺ 407 |
| 47-91 | 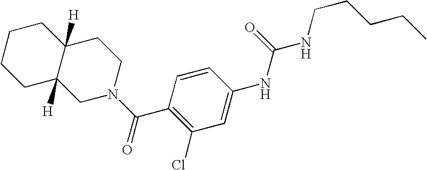 | [M + 1]⁺ 406 |
| 47-92 | 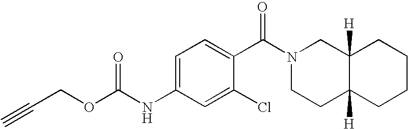 | [M + 1]⁺ 375 |
| 47-93 | 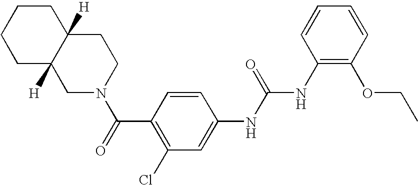 | [M + 1]⁺ 456 |
| 47-94 | 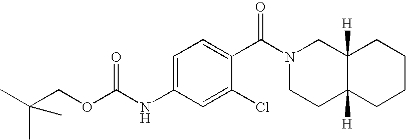 | [M + 1]⁺ 407 |
| 47-95 | 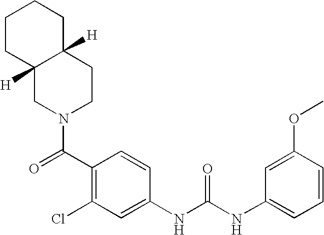 | [M + 1]⁺ 442 |
| 47-96 | 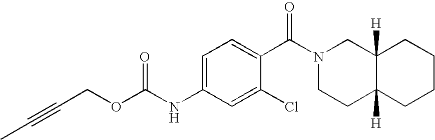 | [M + 1]⁺ 389 |

### EXAMPLE 48

The following compounds are prepared analogously to Example 9 starting from 2-methoxy-4-nitrobenzoyl chloride and either trans or cis decahydroisoquinoline, and treating the intermediate 4-amino-2-methoxybenzamide derivative with the appropriate N-derivatizing agent such as an activated derivative of a carboxylic acid, a chloroformate or an isocyanate.

| Compd | Structure | MS [m/z] |
|---|---|---|
| 48-1 | 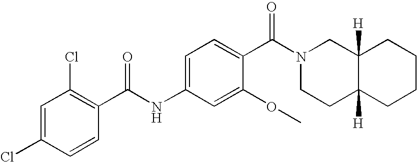 | [M + 1]⁺ 461 |
| 48-2 | 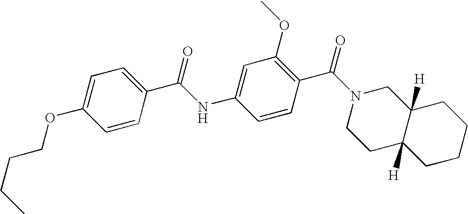 | [M + 1]⁺ 466 |
| 48-3 | 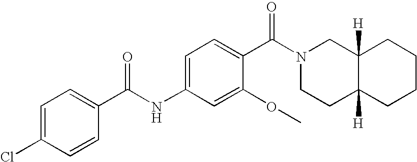 | [M + 1]⁺ 427 |
| 48-4 | 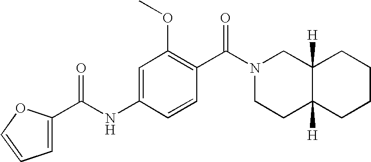 | [M + 1]⁺ 384 |
| 48-5 | 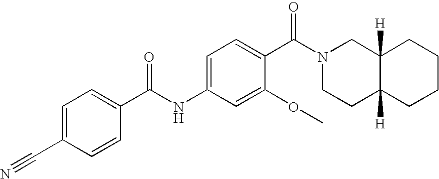 | [M + 1]⁺ 418 |
| 48-6 | 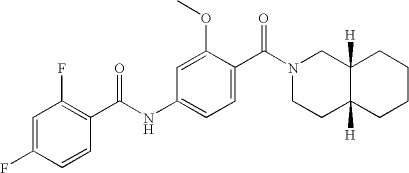 | [M + 1]⁺ 430 |
| 48-7 | 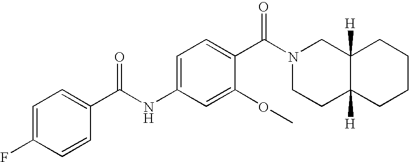 | [M + 1]⁺ 411 |
| 48-8 | 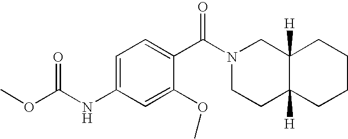 | [M + 1]⁺ 347 |
| 48-9 | 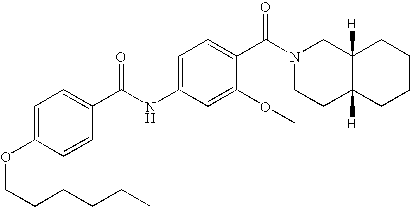 | [M + 1]⁺ 493 |
| 48-10 | 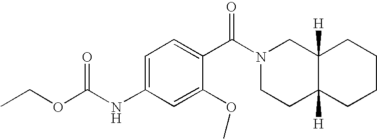 | [M + 1]⁺ 361 |
| 48-11 | 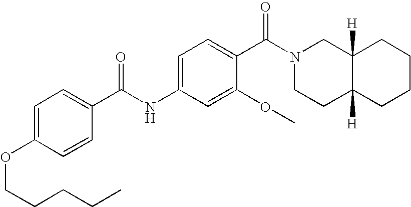 | [M + 1]⁺ 479 |
| 48-12 | 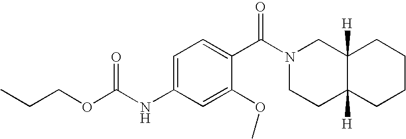 | [M + 1]⁺ 375 |
| 48-13 | 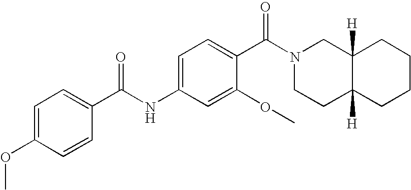 | [M + 1]⁺ 423 |
| 48-14 | 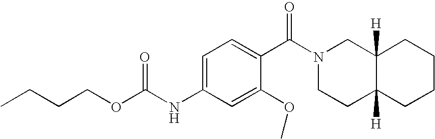 | [M + 1]⁺ 389 |
| 48-15 | 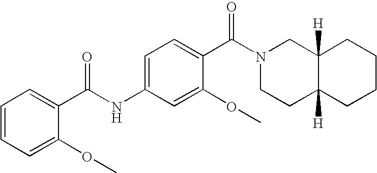 | [M + 1]⁺ 423 |
| 48-16 | 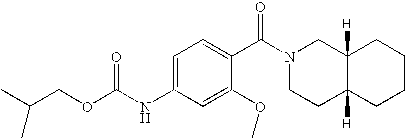 | [M + 1]⁺ 389 |
| 48-17 | 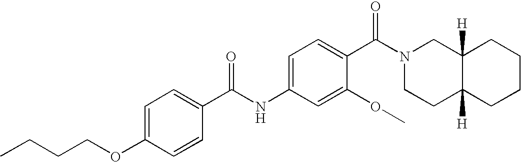 | [M + 1]⁺ 465 |
| 48-18 | 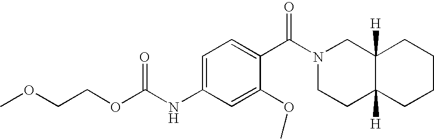 | [M + 1]⁺ 391 |
| 48-19 | 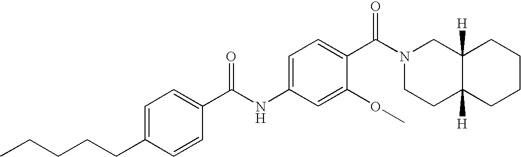 | [M + 1]⁺ 463 |
| 48-20 | 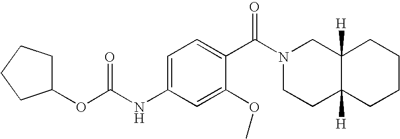 | [M + 1]⁺ 401 |
| 48-21 | 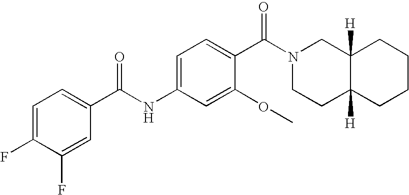 | [M + 1]⁺ 429 |
| 48-22 | 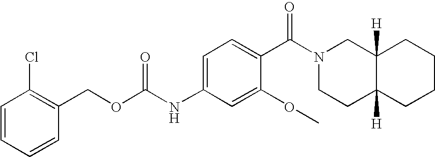 | [M + 1]⁺ 458 |
| 48-23 | 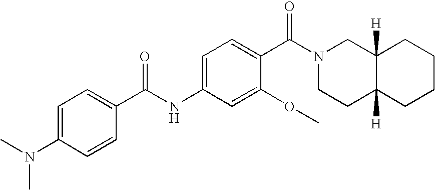 | [M + 1]⁺ 436 |
| 48-24 | 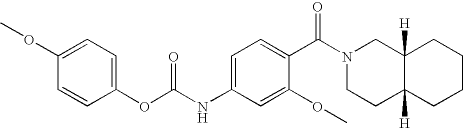 | [M + 1]⁺ 440 |
| 48-25 | 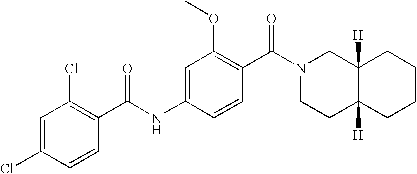 | [M + 1]⁺ 461 |
| 48-26 | 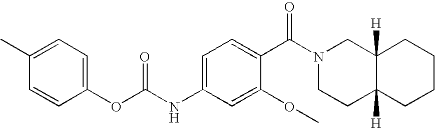 | [M + 1]⁺ 424 |
| 48-27 | 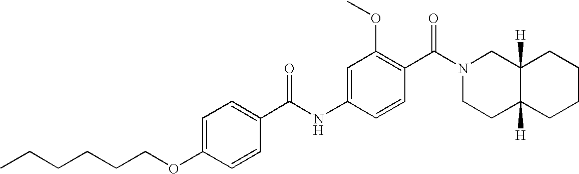 | [M + 1]⁺ 493 |
| 48-28 | 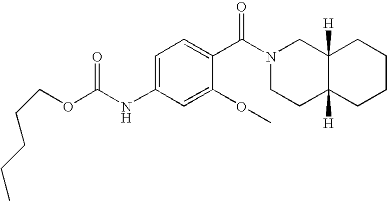 | [M + 1]⁺ 404 |
| 48-29 | 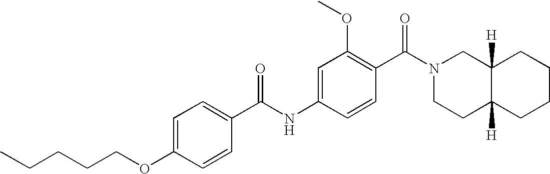 | [M + 1]⁺ 479 |
| 48-30 | 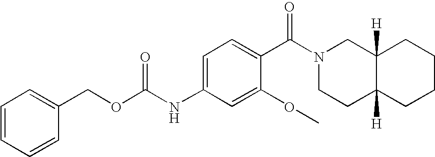 | [M + 1]⁺ 424 |
| 48-31 | 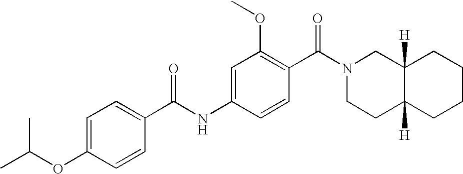 | [M + 1]⁺ 451 |
| 48-32 | 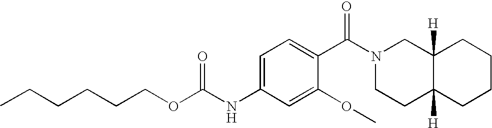 | [M + 1]⁺ 418 |
| 48-33 | 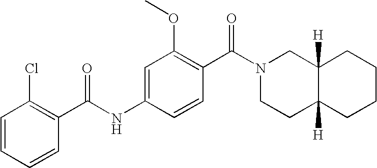 | [M + 1]⁺ 427 |
| 48-34 | 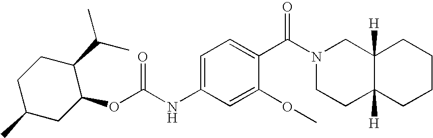 | [M + 1]⁺ 472 |
| 48-35 | 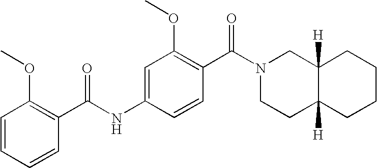 | [M + 1]⁺ 423 |
| 48-36 | 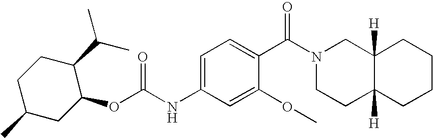 | [M + 1]⁺ 472 |
| 48-37 | 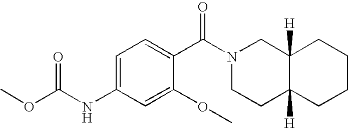 | [M + 1]⁺ 347 |
| 48-38 | 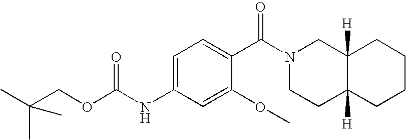 | [M + 1]⁺ 404 |
| 48-39 | 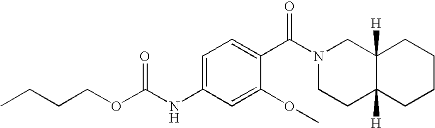 | [M + 1]⁺ 389 |
| 48-40 | 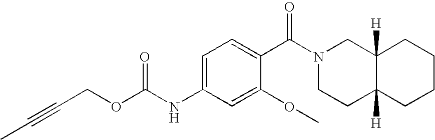 | [M + 1]⁺ 385 |
| 48-41 | 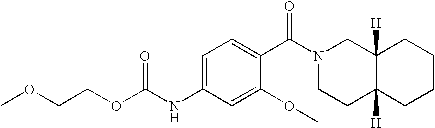 | [M + 1]⁺ 391 |
| 48-42 | 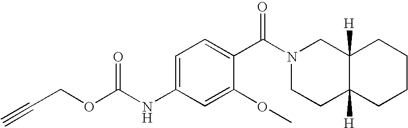 | [M + 1]⁺ 372 |
| 48-43 | 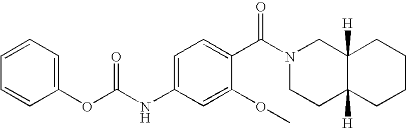 | [M + 1]⁺ 409 |
| 48-44 | 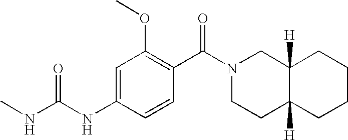 | [M + 1]⁺ 346 |
| 48-45 | 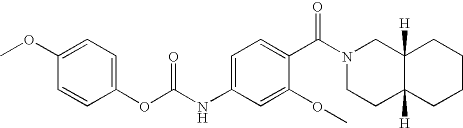 | [M + 1]⁺ 439 |
| 48-46 | 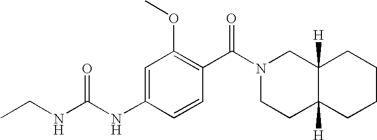 | [M + 1]⁺ 360 |
| 48-47 | 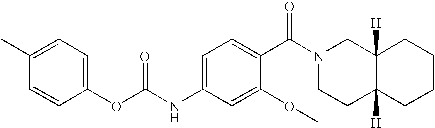 | [M + 1]⁺ 423 |
| 48-48 | 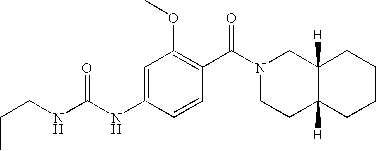 | [M + 1]⁺ 374 |
| 48-49 | 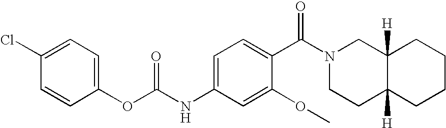 | [M + 1]⁺ 443 |
| 48-50 | 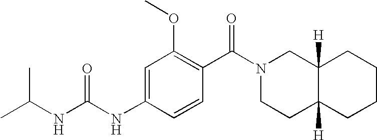 | [M + 1]⁺ 374 |
| 48-51 | 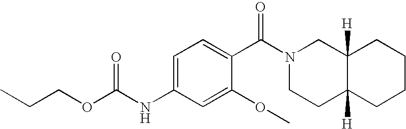 | [M + 1]⁺ 375 |
| 48-52 | 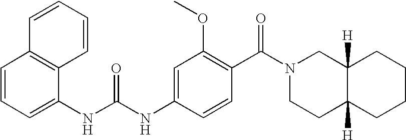 | [M + 1]⁺ 458 |
| 48-53 | 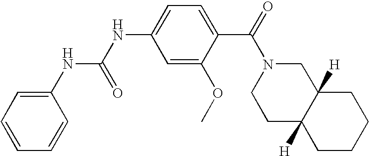 | [M + 1]⁺ 409 |
| 48-54 | 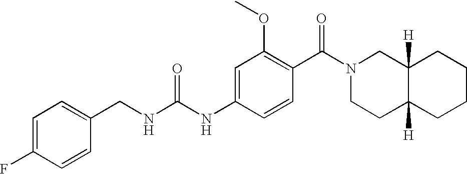 | [M + 1]⁺ 440 |
| 48-55 | 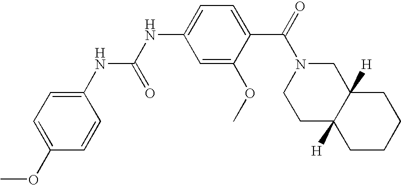 | [M + 1]⁺ 439 |
| 48-56 | 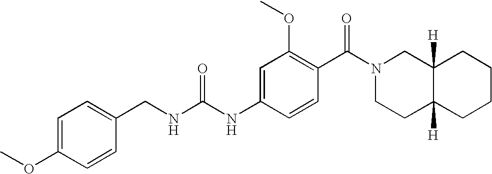 | [M + 1]⁺ 452 |
| 48-57 | 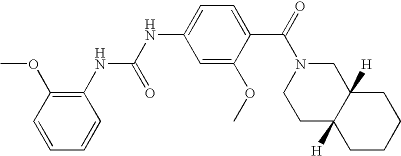 | [M + 1]⁺ 439 |
| 48-58 | 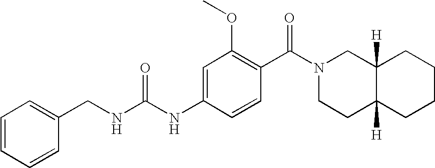 | [M + 1]⁺ 422 |
| 48-59 | 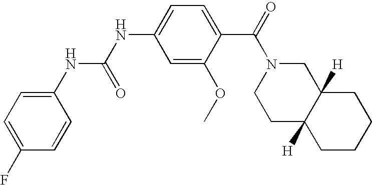 | [M + 1]⁺ 426 |
| 48-60 | 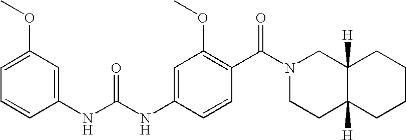 | [M + 1]⁺ 439 |
| 48-61 | 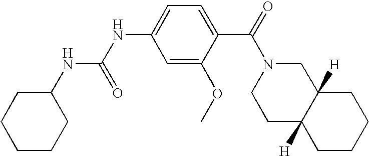 | [M + 1]⁺ 415 |
| 48-62 | 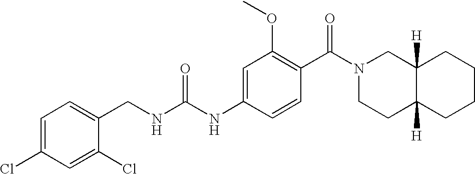 | [M + 1]⁺ 490 |
| 48-63 | 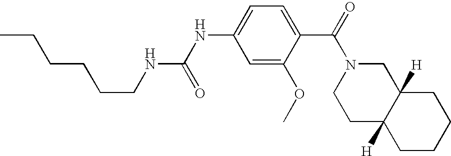 | [M + 1]⁺ 417 |
| 48-64 | 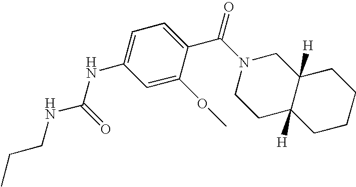 | [M + 1]⁺ 374 |
| 48-65 | 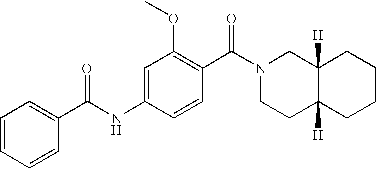 | [M + 1]⁺ 394 |
| 48-66 | 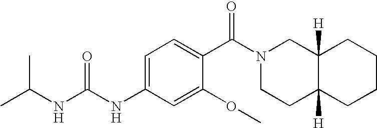 | [M + 1]⁺ 374 |
| 48-67 | 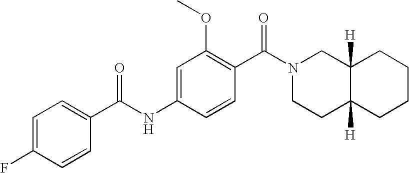 | [M + 1]⁺ 411 |
| 48-68 | 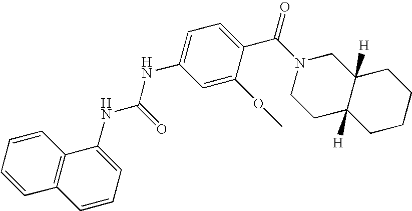 | [M + 1]⁺ 458 |
| 48-69 | 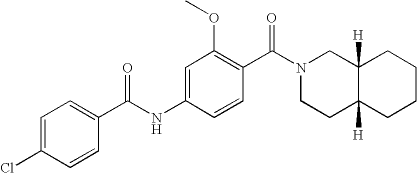 | [M + 1]⁺ 428 |
| 48-70 | 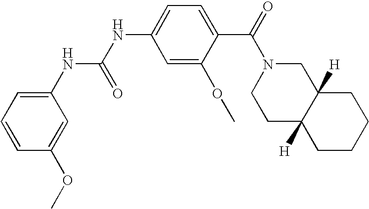 | [M + 1]⁺ 438 |
| 48-71 | 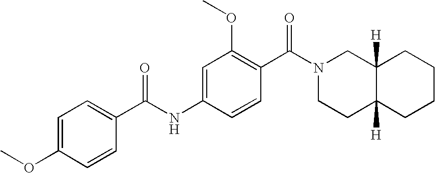 | [M + 1]⁺ 424 |
| 48-72 | 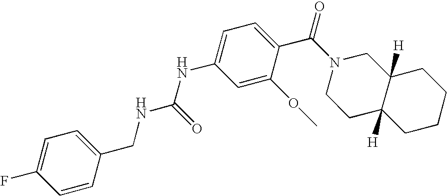 | [M + 1]⁺ 440 |
| 48-73 | 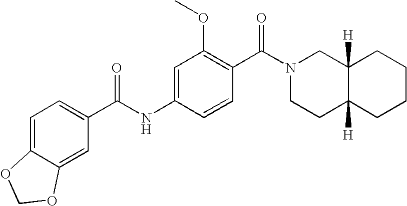 | [M + 1]⁺ 438 |
| 48-74 | 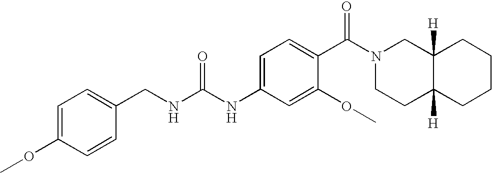 | [M + 1]⁺ 453 |

### EXAMPLE 49

The following compounds are prepared analogously to Example 17.

| | | MS |
|---|---|---|
| Compd | Structure | [m/z] |
| 49-1 | 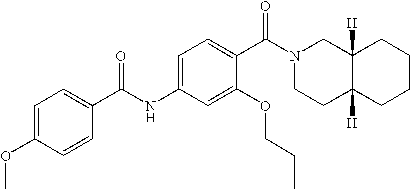 | [M + 1]⁺451 |
| 49-2 | 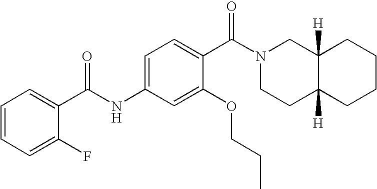 | [M + 1]⁺439 |
| 49-3 | 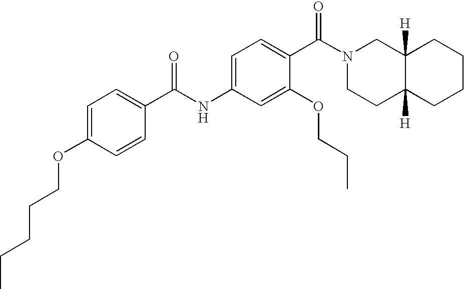 | [M + 1]⁺508 |
| 49-4 | 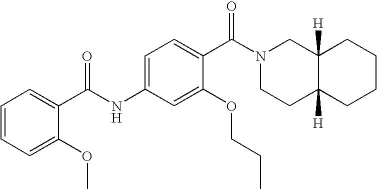 | [M + 1]⁺452 |
| 49-5 | 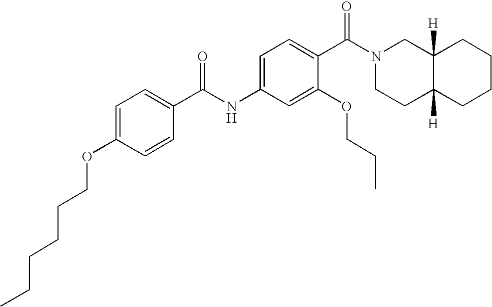 | [M + 1]⁺522 |
| 49-6 | 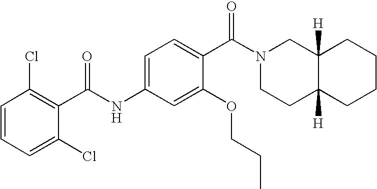 | [M + 1]⁺489 |
| 49-7 | 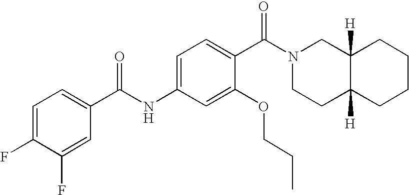 | [M + 1]⁺457 |
| 49-8 | 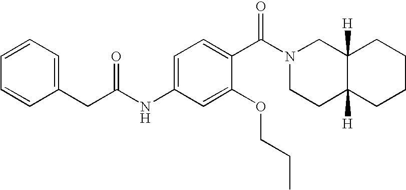 | [M + 1]⁺436 |
| 49-9 | 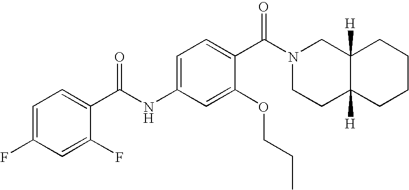 | [M + 1]⁺457 |
| 49-10 | 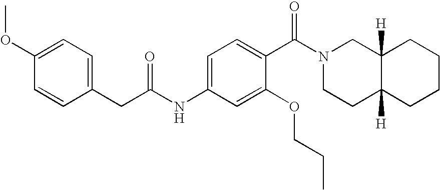 | [M + 1]⁺466 |
| 49-11 | 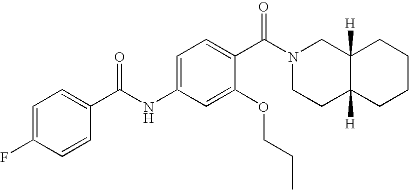 | [M + 1]⁺439 |
| 49-12 | 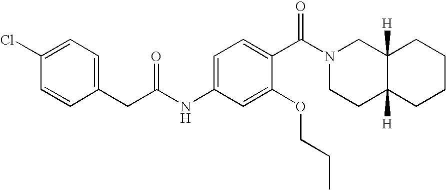 | [M + 1]⁺469 |
| 49-13 | 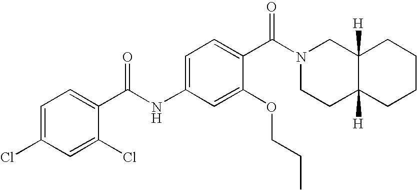 | [M + 1]⁺489 |
| 49-14 | 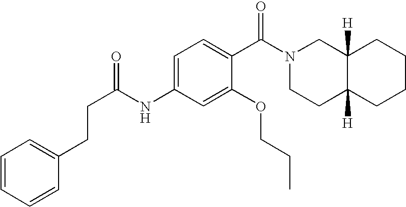 | [M + 1]⁺450 |
| 49-15 | 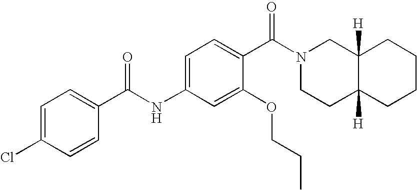 | [M + 1]⁺455 |
| 49-16 | 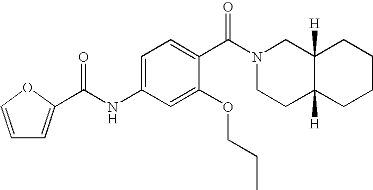 | [M + 1]⁺411 |

### EXAMPLE 50

The following compounds are prepared analogously to Example 17.

| | | MS |
|---|---|---|
| Compd | Structure | [m/z] |
| 50-1 | 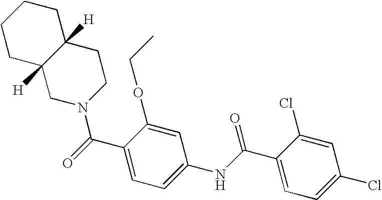 | [M + 1]⁺475 |
| 50-2 | 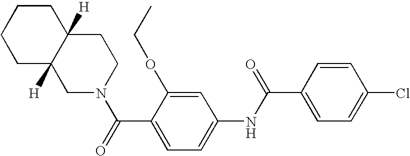 | [M + 1]⁺441 |
| 50-3 | 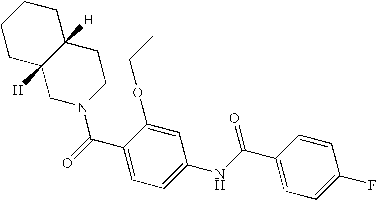 | [M + 1]⁺425 |
| 50-4 | 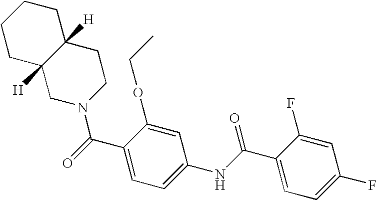 | [M + 1]⁺443 |
| 50-5 | 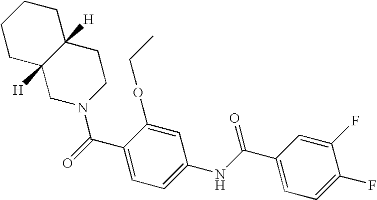 | [M + 1]⁺443 |

### EXAMPLE 51

### 5-((4aS,8aR)-Octahydro-isoquinoline-2-carbonyl)-2,3-dihydro-indole-1-carboxylic acid tert-butyl ester

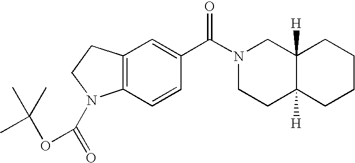

The title compound is prepared analogously to Example 32: API-MS 385 [M+1]⁺.

### EXAMPLE 52

### 5-((4aR,8aR)-Octahydro-isoquinoline-2-carbonyl)-2,3-dihydro-indole-1-carboxylic acid tert-butyl ester

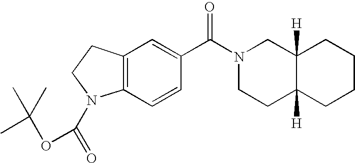

The title compound is prepared analogously to Example 32: API-MS 385 [M+1]⁺.

### EXAMPLE 53

### 2,4-Dichloro-N-(2-cyclohexyl-1-oxo-2,3-dihydro-1H-isoindol-5-yl)-benzamide

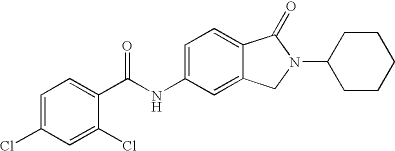

### A. 4-Bromo-2-methylbenzoic acid methyl ester

A mixture of 4-bromo-2-methylbenzoic acid (16.0 g, 74.4 mmol), 1 mL conc. H₂SO₄, and 100 mL MeOH is heated to reflux for 5 h. The reaction is cooled to RT, and the solvent is removed by rotary evaporation. The residue is taken up in EtOAc and washed sequentially with water, saturated aqueous Na₂CO₃, water, and brine. The organic layer is dried over anhydrous Na₂SO₄, and concentrated. Purification by chromatography on silica (eluent: 5% EtOAc in hexane) affordes 4-bromo-2-methylbenzoic acid methyl ester as a colorless oil: NMR (CDCl₃) 2.58 (s, 3H), 3.88 (s, 3H), 7.36-7.42 (m, 2H), 7.77 (d, 1H, J=8.3).

### B. 4-Bromo-2-bromomethyl-benzoic acid methyl ester

A solution of the title A compound, 4-bromo-2-methylbenzoic acid methyl ester (1.14 g, 15.0 mmol) in 20 mL carbontetrachloride is treated sequentially with N-bromosuccinimide (1.06 g, 18.0 mmol) and benzoyl peroxide (100 mg). The reaction is heated at reflux for 4.5 h, then cooled to RT, and partitioned between water and EtOAc. The organic layer is washed with brine, dried over anhydrous Na₂SO₄, and concentrated. Purification by chromatography on silica (eluent: 5% EtOAc in hexane) affordes 4-bromo-2-bromomethyl-benzoic acid methyl ester as a white solid: NMR (CDCl₃) 3.94 (s, 3H), 4.90 (s, 2H), 7.51 (dd, 1H, J=8.7, 1.9), 7.63 (d, 1H, J=1.9), 7.85 (d, 1H, J=8.7).

### C. 5-Bromo-2-cyclohexyl-2,3-dihydro-isoindol-1-one

A solution of the title B compound, 4-bromo-2-bromomethyl-benzoic acid methyl ester (700 mg, 2.27 mmol) in 20 mL DMF is treated sequentially with cyclohexylamine (0.31 mL, 2.72 mmol) and diisopropylethylamine (0.79 mL, 4.54 mmol). The reaction is heated at 50° C. for 4 h, cooled to RT, and partitioned between EtOAc and water. The organic layer is washed sequentially with saturated aqueous lithium chloride and brine, dried over anhydrous Na₂SO₄, and concentrated. Purification by chromatography on silica (eluent: 30% EtOAc in hexane) affordes 5-bromo-2-cyclohexyl-2,3-dihydro-isoindol-1-one as a yellow solid: NMR (CDCl₃) 1.14-1.19 (m, 1H), 1.43-1.50 (m, 4H), 1.71-1.75 (br d, 1H, J=12.8), 1.85-1.88 (m, 4H), 4.22-4.25 (m, 1H), 4.33 (s, 2H), 7.65 (dd, 3H, J=34.6, 7.4).

### D. 5-Amino-2-cyclohexyl-2,3-dihydro-isoindol-1-one

A flask is charged with the title C compound, 5-bromo-2-cyclohexyl-2,3-dihydro-isoindol-1-one (350 mg, 1.195 mmol), tris(dibenzylidineacetone)dipalladium(0) (27 mg, 0.0299 mmol) and (R)-(+)-2,2′-bis(diphenylphosphino)-1,1′-binaphthyl (56 mg, 0.0896 mmol), then purged with nitrogen. The contents are dissolved in 10 mL of toluene and benzophenone imine (0.24 mL, 1.43 mmol) and sodium t-butoxide (139 mg, 1.43 mmol) are added. The reaction mixture is degassed, and then heated at 100° C. for 2 h. Toluene is removed by rotary evaporation, and the residue is dissolved in 10 mL of THF. The solution is treated with with 1 N aqueous HCl (5 mL, 5.00 mmol) and stirred at RT for 2 h. The reaction is made basic with 1 N aqueous NaOH, and partitioned between EtOAc and water. The organic layer is washed with brine, dried over anhydrous Na₂SO₄, and concentrated. Purification by chromatography on silica (eluent: EtOAc) affordes 5-amino-2-cyclohexyl-2,3-dihydro-isoindol-1-one as a yellow solid: NMR (CDCl₃) 1.22-1.26 (m, 1H), 1.41-1.51 (m, 4H), 1.71 (br d, 1H, J=13.6), 1.83-1.85 (m, 4H), 3.97 (s, 2H), 4.16-4.23 (m, 3H), 6.67-6327 (m, 2H), 7.61 (d, 1H, J=8.3).

### E. 2,4-Dichloro-N-(2-cyclohexyl-1-oxo-2,3-dihydro-1H-isoindol-5-yl)-benzamide

Under parallel reaction synthesis conditions, solutions of NMM (2.0 M in THF, 126 μL, 0.252 mmol) and 2,4-dichlorobenzoyl chloride (1.0 M in THF, 222 μL, 0.220 mmol) are dispensed sequentially into a vial containing a solution of the title D compound, 5-amino-2-cyclohexyl-2,3-dihydro-isoindol-1-one (0.387 M in 3:1 THF/DMF, 400 μL, 0.148 mmol). The vial is shaken at RT for 16 h. The reaction mixture is acidified with 50 μL of TFA and purified by HPLC to afford 2,4-dichloro-N-(2-cyclohexyl-1-oxo-2,3-dihydro-1H-isoindol-5-yl)-benzamide: API-MS 403 [M+1]⁺.

### EXAMPLE 54

The following compounds are prepared analogously to Example 53.

| | | MS |
|---|---|---|
| Compd | Structure | [m/z] |
| 55-1 | 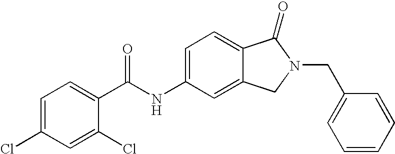 | [M + 1]⁺411 |
| 55-2 | 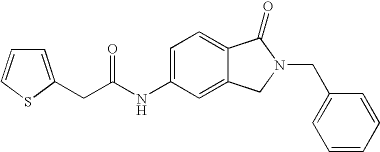 | [M + 1]⁺363 |
| 55-3 | 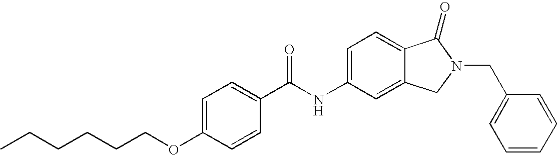 | [M + 1]⁺443 |
| 55-4 | 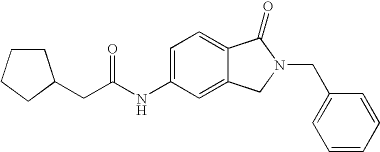 | [M + 1]⁺349 |
| 55-5 | 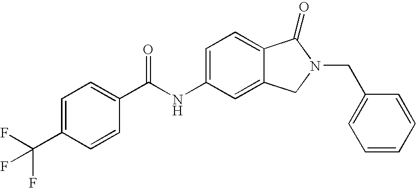 | [M + 1]⁺411 |
| 55-6 | 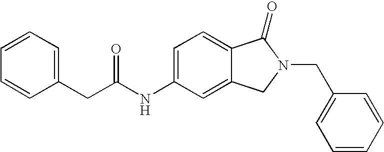 | [M + 1]⁺357 |
| 55-7 | 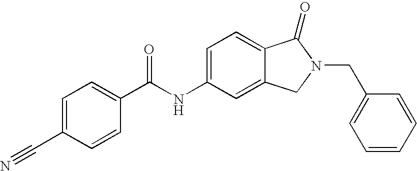 | [M + 1]⁺368 |
| 55-8 | 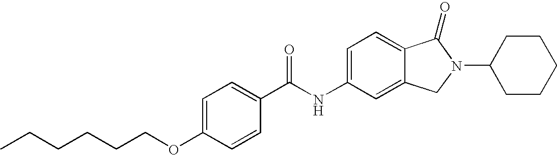 | [M + 1]⁺435 |
| 55-9 | 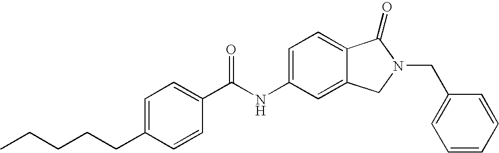 | [M + 1]⁺413 |
| 55-10 | 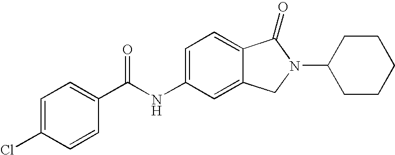 | [M + 1]⁺369 |
| 55-11 | 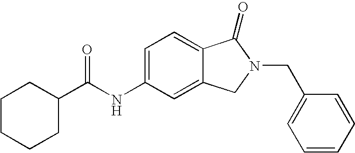 | [M + 1]⁺349 |
| 55-12 | 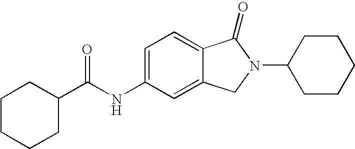 | [M + 1]⁺341 |
| 55-13 | 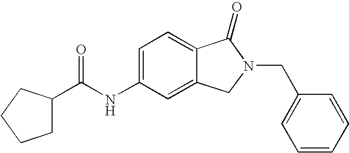 | [M + 1]⁺335 |
| 55-14 | 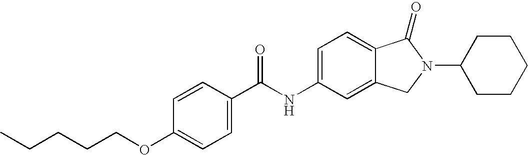 | [M + 1]⁺421 |
| 55-15 | 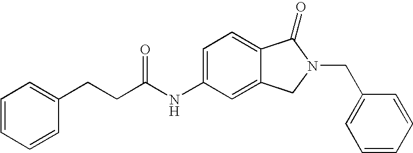 | [M + 1]⁺371 |
| 55-16 | 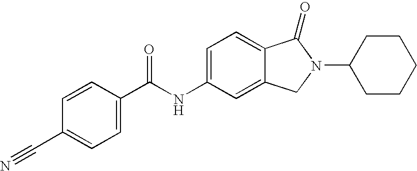 | [M + 1]⁺360 |
| 55-17 | 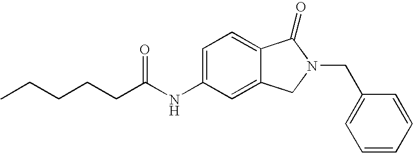 | [M + 1]⁺337 |
| 55-18 | 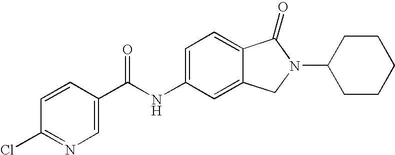 | [M + 1]⁺370 |
| 55-19 | 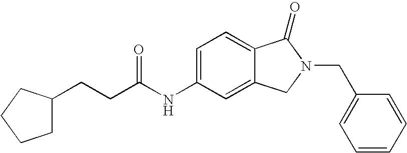 | [M + 1]⁺363 |
| 55-20 | 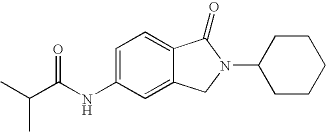 | [M + 1]⁺301 |
| 55-21 | 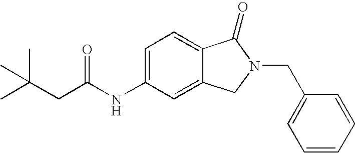 | [M + 1]⁺337 |

### EXAMPLE 55

### N-(2-Benzyl-1-oxo-1,2,3,4-tetrahydro-isoquinolin-6-yl)-2,4-dichloro-benzamide

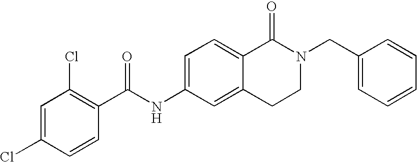

### A. 6-Bromo-3,4-dihydro-2H-isoquinolin-1-one

A solution of 5-bromo-indan-1-one (4.22 g, 20 mmol) in 50 mL of TFA is treated with trimethylsilylazide (4 mL, 30 mmol) at RT. After 7 days, the reaction is quenched with ice, then diluted with water while stirring. The precipitated solid is collected by vacuum filtration and 6-bromo-3,4-dihydro-2H-isoquinolin-1-one is isolated by chromatography on silica (eluent: EtOAc/hexane-3/2 EtOAc).

### B. 2-Benzyl-6-bromo-3,4-dihydro-2H-isoquinolin-1-one

To a solution of the title A compound, 6-bromo-3,4-dihydro-2H-isoquinolin-1-one (570 mg, 2.52 mmol) and benzyl bromide (390 μL, 3.28 mmol) in 10 mL of DMF is added sodium hydride (131 mg, 3.28 mmol), and the reaction is stirred at RT for 3 h. The reaction is quenched with 1 N aqueous HCl, and the product is taken up in EtOAc. The organic solution is washed with water and brine, dried over anhydrous Na₂SO₄ and concentrated. Chromatography on silica (eluent: EtOAc/hexane—1/4) affords 2-benzyl-6-bromo-3,4-dihydro-2H-isoquinolin-1-one.

### C. 6-Amino-2-benzyl-3,4-dihydro-2H-isoquinolin-1-one

A flask is charged with the title B compound, 2-benzyl-6-bromo-3,4-dihydro-2H-isoquinolin-1-one (740 mg, 2.3 mmol), tris(dibenzylidineacetone)dipalladium(0) (5 mg, 0.0059 mmol) and (R)-(+)-2,2′-bis(diphenylphosphino)-1,1′-binaphthyl (11 mg, 0.0173 mmol), then purged with nitrogen. The contents are dissolved in 15 mL of toluene and benzophenone imine (0.24 mL, 1.43 mmol) and sodium t-butoxide (309 mg, 3.22 mmol) are added. The reaction mixture is degassed, and then heated at 90° C. for 3 h. Toluene is removed by rotary evaporation, and the residue is dissolved in 15 mL of THF/water—4/1. The solution is treated with with 1 N aqueous HCl (10 mL, 10 mmol) and stirred at RT for 1 h. The reaction is quenched with 1 N aqueous NaOH, and partitioned between EtOAc and water. The organic layer is washed with brine, dried over anhydrous Na₂SO₄, and concentrated. Purification by chromatography on silica (eluent: EtOAc/hexane—3/2) affordes 6-amino-2-benzyl-3,4-dihydro-2H-isoquinolin-1-one as a light grey solid: NMR (CDCl₃) 1.22-1.26 (m, 1H), 1.41-1.51 (m, 4H), 1.71 (br d, 1H, J=13.6), 1.83-1.85 (m, 4H), 3.97 (s, 2H), 4.16-4.23 (m, 3H), 6.67-6.72 (m, 2H), 7.61 (d, 1H, J=8.3).

### D. N-(2-Benzyl-1-oxo-1,2,3,4-tetrahydro-isoquinolin-6-yl)-2,4-dichloro-benzamide

Under parallel reaction synthesis conditions, solutions of NMM (2.0 M in THF, 126 μL, 0.252 mmol) and 2,4-dichlorobenzoyl chloride (1.0 M in THF, 222 μL, 0.220 mmol) are dispensed sequentially into a vial containing a solution of the title C compound, 6-amino-2-benzyl-3,4-dihydro-2H-isoquinolin-1-one (0.387 M in 3:1 THF/DMF, 400 μL, 0.148 mmol). The vial is shaken at RT for 16 h. The reaction mixture is acidified with 50 μL of TFA and purified by HPLC to afford N-(2-benzyl-1-oxo-1,2,3,4-tetrahydro-isoquinolin-6-yl)-2,4-dichloro-benzamide; API-MS 425 [M+1]⁺.

### EXAMPLE 56

The following compounds are prepared analogously to Example 55.

| | | MS |
|---|---|---|
| Compd | Structure | [m/z] |
| 56-1 | 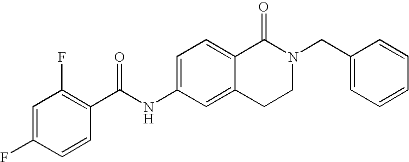 | [M + 1]⁺393 |
| 56-2 | 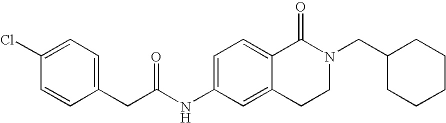 | [M + 1]⁺411 |
| 56-3 | 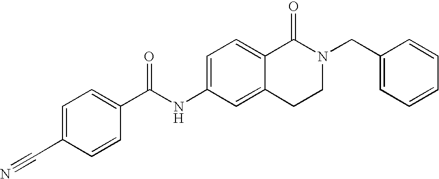 | [M + 1]⁺382 |
| 56-4 | 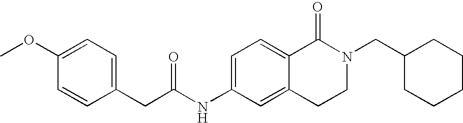 | [M + 1]⁺407 |
| 56-5 | 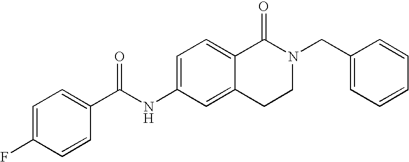 | [M + 1]⁺375 |
| 56-6 | 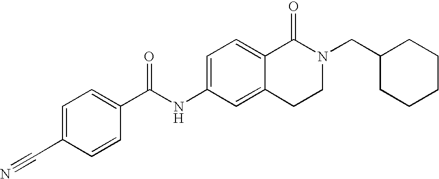 | [M + 1]⁺388 |
| 56-7 | 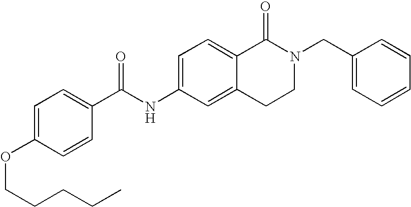 | [M + 1]⁺443 |
| 56-8 | 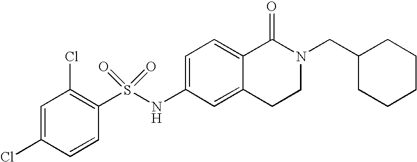 | [M + 1]⁺467 |
| 56-9 | 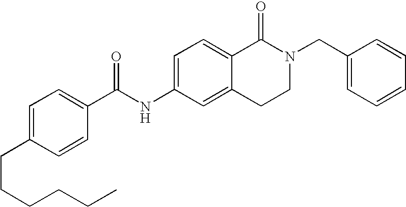 | [M + 1]⁺441 |
| 56-10 | 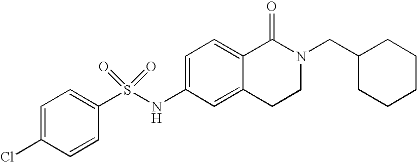 | [M + 1]⁺433 |
| 56-11 | 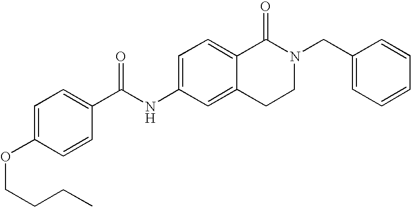 | [M + 1]⁺429 |
| 56-12 | 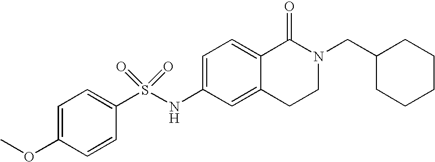 | [M + 1]⁺429 |
| 56-13 | 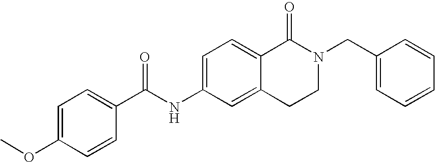 | [M + 1]⁺387 |
| 56-14 | 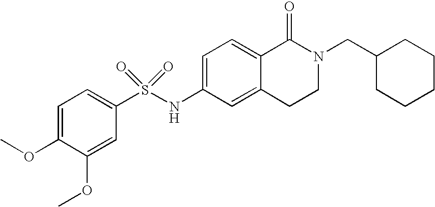 | [M + 1]⁺459 |
| 56-15 | 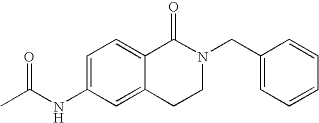 | [M + 1]⁺295 |
| 56-16 | 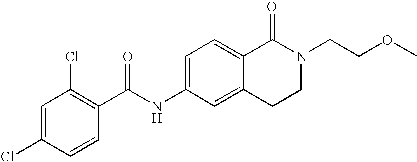 | [M + 1]⁺393 |
| 56-17 | 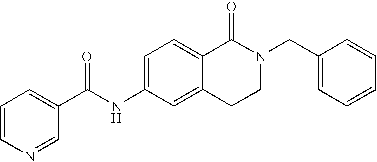 | [M + 1]⁺358 |
| 56-18 | 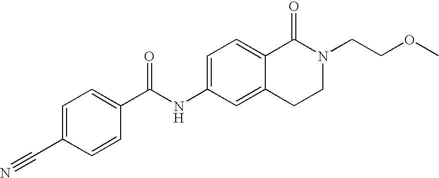 | [M + 1]⁺350 |
| 56-19 | 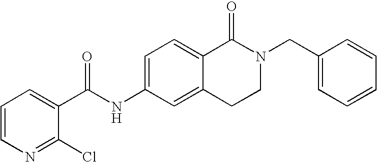 | [M + 1]⁺392 |
| 56-20 | 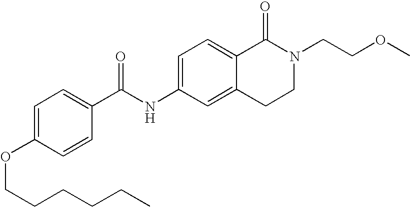 | [M + 1]⁺425 |
| 56-21 | 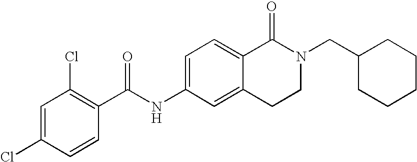 | [M + 1]⁺431 |
| 56-22 | 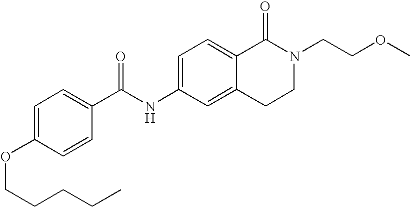 | [M + 1]⁺411 |
| 56-23 | 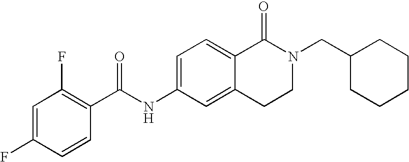 | [M + 1]⁺399 |
| 56-24 | 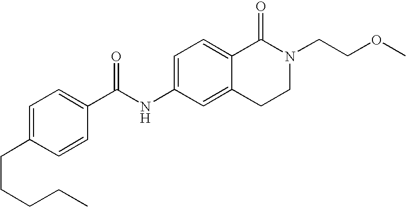 | [M + 1]⁺395 |
| 56-25 | 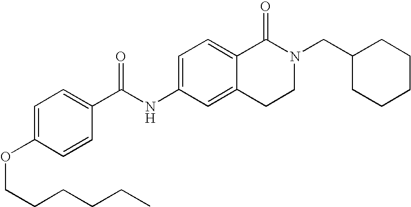 | [M + 1]⁺463 |
| 56-26 | 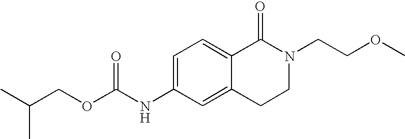 | [M + 1]⁺321 |
| 56-27 | 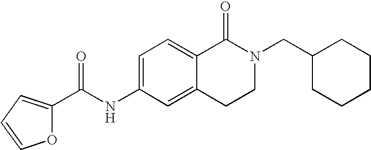 | [M + 1]⁺353 |
| 56-28 | 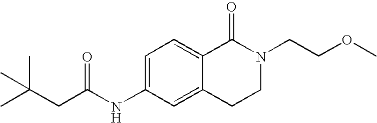 | [M + 1]⁺391 |

### EXAMPLE 57

### Cyclohexylmethyl-7-(2-fluoro-benzyloxy)-2,3,4,5-tetrahydro-2-benzazepin-1-one

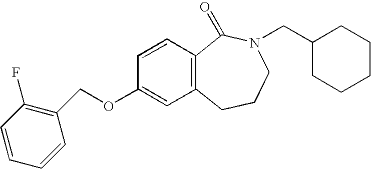

### A. 7-Methoxy-2,3,4,5-tetrahydro-2-benzazepin-1-one

A solution of 6-methoxy-3,4-dihydro-2H-naphthalen-1-one (5.289 g, 30 mmol) in 50 mL of TFA is treated with trimethylsilylazide (5.2 ml, 39 mmol) at RT. After 7 days, the reaction is quenched with ice, then diluted with water while stirring. The product is taken up in EtOAc, and the organic solution is washed with aqueous saturated Na₂CO₃ and brine, dried over anhydrous Na₂SO₄ and concentrated. The product is purified by chromatography on silica (eluent: EtOAc/hexane—2/3→EtOAc) to afford 7-methoxy-2,3,4,5-tetrahydro-2-benzazepin-1-one: API-MS 192 [M+1]⁺.

### B. 2-Cyclohexylmethyl-7-methoxy-2,3,4,5-tetrahydro-2-benzazepin-1-one

To a solution of the title A compound, 7-methoxy-2,3,4,5-tetrahydro-2-benzazepin-1-one (1.6 g, 8.38 mmol) and cyclohexylmethylbromide (1.8 mL, 12.57 mmol) in 20 mL of DMF is added sodium hydride (470 mg, 11.73 mmol) followed by tetraethylammonium iodide (861 mg, 3.35 mmol), and the reaction is stirred at RT for 48 h. The reaction is quenched with 1 N aqueous HCl, and the product is taken up in EtOAc. The organic solution is washed with water and brine, dried over anhydrous Na₂SO₄ and concentrated. Chromatography on silica (eluent: EtOAc/hexane—1/4) affords 2-cyclohexyl-methyl-7-methoxy-2,3,4,5-tetrahydro-2-benzazepin-1-one: API-MS 288 [M+1]⁺.

### C. 2-Cyclohexylmethyl-7-hydroxy-2,3,4,5-tetrahydro-2-benzazepin-1-one

A mixture of the title B compound, 2-cyclohexyl-methyl-7-methoxy-2,3,4,5-tetrahydro-2-benzazepin-1-one (2.3 g, 8.01 mmol), 20 mL of acetic acid and 20 mL of aqueous 48% hydrobromic acid is heated at 110° C. for 24 h. The reaction is cooled to RT and diluted with water (40 mL), and the precipitated product is collected by vacuum filtration, washed with water and dried to afford 2-cyclohexylmethyl-7-hydroxy-2,3,4,5-tetrahydro-2-benzazepin-1-one: API-MS 274 [M+1]⁺.

### D. Cyclohexylmethyl-7-(2-fluoro-benzyloxy)-2,3,4,5-tetrahydro-2-benzazepin-1-one

Under parallel reaction synthesis conditions, a solution of 2-fluorobenzyl bromide (1.0 M in THF, 222 μL, 0.220 mmol) is dispensed into a vial containing a solution of the title O compound, 2-cyclohexylmethyl-7-hydroxy-2,3,4,5-tetrahydro-2-benzazepin-1-one (0.387 M in 3:1 THF/DMF, 400 μL, 0.148 mmol) followed by addition of cesium carbonate (96 mg, 0.296 mmol). The vial is shaken at RT for 16 h and the solids are removed by filtration. The filtrate is acidified with 50 μL of TFA and purified by HPLC to afford cyclohexylmethyl-7-(2-fluoro-benzyloxy)-2,3,4,5-tetrahydro-2-benzazepin-1-one: API-MS 382 [M+1]⁺.

### EXAMPLE 58

The following compounds are prepared analogously to Example 57 by treating the title C compound in Example 57 with the appropriate alkylating agent.

| | | MS |
|---|---|---|
| Compd | Structure | [m/z] |
| 58-1 | 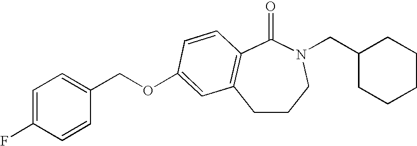 | [M + 1]⁺382 |
| 58-2 | 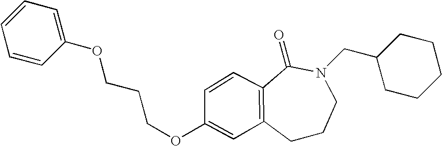 | [M + 1]⁺409 |
| 58-3 | 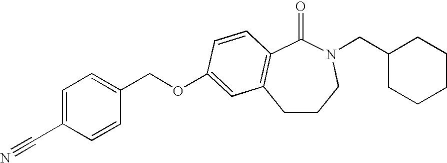 | [M + 1]⁺389 |
| 58-4 | 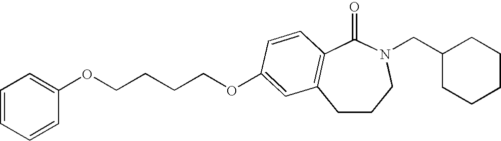 | [M + 1]⁺423 |
| 58-5 | 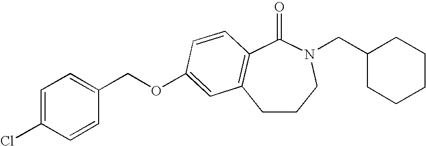 | [M + 1]⁺398 |
| 58-6 | 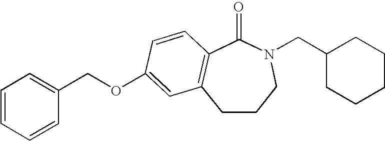 | [M + 1]⁺364 |
| 58-7 | 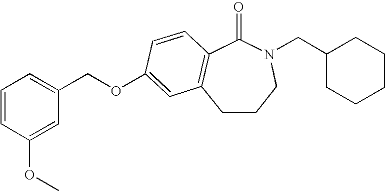 | [M + 1]⁺395 |
| 58-8 | 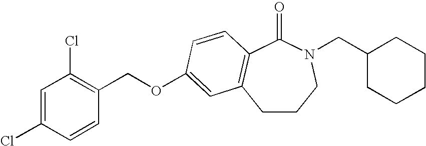 | [M + 1]⁺432 |
| 58-9 | 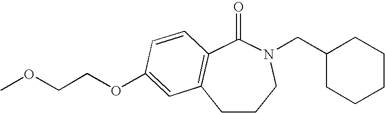 | [M + 1]⁺332 |
| 58-10 | 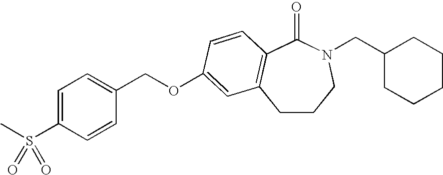 | [M + 1]⁺443 |
| 58-11 | 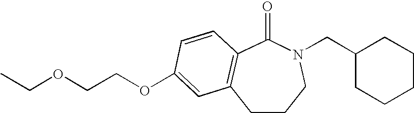 | [M + 1]⁺346 |
| 58-12 | 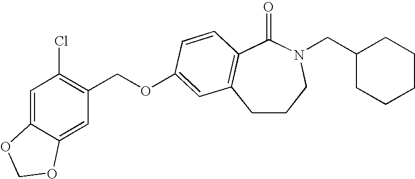 | [M + 1]⁺443 |
| 58-13 | 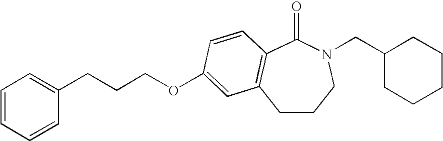 | [M + 1]⁺393 |
| 58-14 | 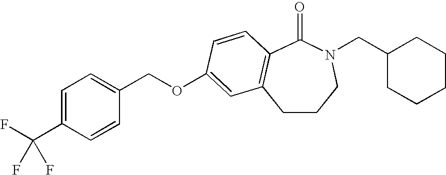 | [M + 1]⁺433 |
| 58-15 | 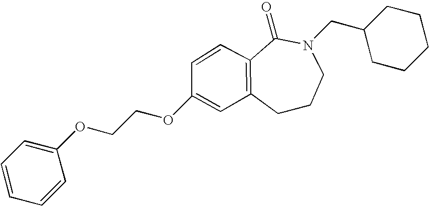 | [M + 1]⁺395 |
| 58-16 | 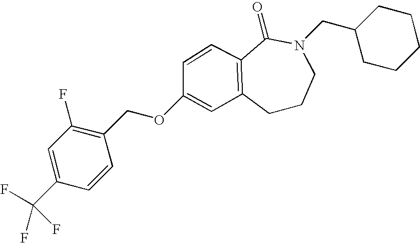 | [M + 1]⁺451 |

1. A compound according to the formula 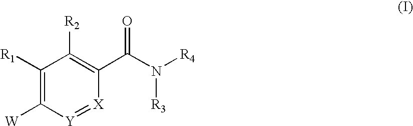
wherein
R₁ and R₂ are independently hydrogen, cyano, halo, nitro, trifluoromethyl, optionally substituted amino, alkyl, alkoxy, aryl, aralkyl, heteroaryl or heteroaralkyl; or
R₁ and R₂ combined together with the carbon atoms they are attached to form an optionally substituted 5- to 7-membered aromatic or heteroaromatic ring;
R₃ is optionally substituted lower alkyl; or
R₃ and R₂ combined together with the amide group to which R₃ is attached and the carbon atoms to which R₂ and the amide are attached form an optionally substituted 5- to 7-membered carbocyclic or heterocyclic ring;
R₄ is optionally substituted alkyl, cycloalkyl, heterocyclyl, aryl, aralkyl or heteroaralkyl; or
R₄ and R₃ taken together with the nitrogen atom to which they are attached form a 5- to 8-membered ring which may be optionally substituted or may contain another heteroatom selected from oxygen, nitrogen and sulfur; or
R₄ and R₃ taken together with the nitrogen atom to which they are attached form a 8- to 12-membered fused bicyclic ring, which may be optionally substituted or may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen, optionally substituted alkyl or aralkyl; or
R₅ and R₁ are optionally substituted alkylene which combined together with the nitrogen atom to which R₅ is attached and the carbon atoms to which W and R₁ are attached form a 5- or 6-membered ring;
R₆ is optionally substituted alkyl, cycloalkyl, heterocyclyl, aryl, aralkyl or heteroaralkyl;
R₈ is optionally substituted alkyl, aralkyl or heteroaralkyl;
R₉ is hydrogen, optionally substituted alkyl, cycloalkyl, heterocyclyl, heterocycloalkyl, aralkyl, heteroaralkyl, alkanoyl, aroyl or heteroaroyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic or heteroaromatic ring optionally substituted with alkyl, alkoxy, aryl, heteroaryl, halo, —NR₅Z, —C(O)NR₆R₇, —OR₉ or —OC(O)NR₆R₇;
X and Y are independently CH or nitrogen; or
—X═Y— if —CH₂—, oxygen, sulfur or —NR₁₀— in which R₁₀ is hydrogen or lower alkyl;
or a pharmaceutically acceptable salt thereof.

2. A compound according to claim 1 wherein
R₁ and R₂ are independently hydrogen, halo, optionally substituted amino, lower alkyl or lower alkoxy; or
R₁ and R₂ combined together with the carbon atoms they are attached to form an optionally substituted 6-membered aromatic ring;
R₃ is lower alkyl; or
R₃ and R₂ combined together with the amide group to which R₃ is attached and the carbon atoms to which R₂ and the amide are attached form an optionally substituted 5- to 7-membered carbocyclic or heterocyclic ring;
R₄ is optionally substituted alkyl, cycloalkyl, heterocyclyl, aryl, aralkyl or heteroaralkyl; or
R₄ and R₃ taken together with the nitrogen atom to which they are attached form a fully saturated optionally substituted 6-membered ring; or
R₄ and R₃ taken together with the nitrogen atom to which they are attached form a fully saturated 10-membered fused bicyclic ring, which may be optionally substituted or may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R, —C(O)NR₆R₇, —OR₉ or —OC(O)NR₆R₇ in which
R₅ and R₇ are independently hydrogen or lower alkyl; or
R₅ and R₁ are optionally substituted alkylene which combined together with the nitrogen atom to which R₅ is attached and the carbon atoms to which W and R₁ are attached form a 5-membered ring;
R₆ is optionally substituted alkyl, cycloalkyl, aryl, heteroaryl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇;
X and Y are independently CH or nitrogen; or
—X═Y— is —CH₂—, oxygen, sulfur or —NR₁₀— in which R₁₀ is hydrogen or lower alkyl;
or a pharmaceutically acceptable salt thereof.

3. A compound according to claim 2 wherein
R₁ and R₂ are independently hydrogen, halo, optionally substituted amino, lower alkyl or lower alkoxy; or
R₁ and R₂ combined together with the carbon atoms they are attached to form an optionally substituted 6-membered aromatic ring;
R₃ is methyl or ethyl; or
R₃ and R₂ combined together with the amide group to which R₃ is attached and the carbon atoms to which R₂ and the amide are attached form a 5- to 7-membered carbocyclic ring;
R₄ is —(CHR₁₁)ₙR₁₂ in which
n is zero or an integer from 1 to 3;
R₁₁ is hydrogen, hydroxy or optionally substituted lower alkyl;
R₁₂ is aryl, heterocyclyl or cycloalkyl; or
R₄ and R₃ taken together with the nitrogen atom to which they are attached form an optionally substituted decahydroquinoline or decahydroisoquinoline which may contain another heteroatom selected from oxygen, nitrogen and sulfur;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl; or
R₅ and R₁ are alkylene which combined together with the nitrogen atom to which R₅ is attached and the carbon atoms to which W and R₁ are attached form a 5-membered ring;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl; or
W is optionally substituted aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇;
X is CH;
Y is CH or nitrogen; or
—X═Y— is —CH₂—, oxygen, sulfur or —NR₁₀— in which R₁₀ is hydrogen or methyl;
or a pharmaceutically acceptable salt thereof.

4. A compound according to claim 3 wherein
R₁ and R₂ are independently hydrogen, halo, lower alkyl or lower alkoxy; or
R₁ and R₂ combined together with the carbon atoms they are attached to form an optionally substituted 6-membered aromatic ring;
R₃ is methyl or ethyl;
R₄ is —(CHR₁₁)ₙR₁₂ in which
n is zero or an integer of 1;
R₁₂ is hydrogen;
R₁₂ is optionally substituted cyclohexyl; or R₁₂ is optionally substituted 1-adamantyl providing that n is an integer of 1;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl; or
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇;
X is CH;
Y is CH or nitrogen; or
—X═Y— is —CH₂—, oxygen, sulfur or —NR₁₀— in which R₁₀ is hydrogen or methyl;
or a pharmaceutically acceptable salt thereof.

5. A compound according to claim 4 wherein
R₁ is hydrogen;
R₂ is hydrogen, chloro or methoxy;
R₃ is methyl; R₄ is —(CHR₁₁)ₙR₁₂ in which
n is zero or an integer of 1;
R₁₁ is hydrogen;
R₁₂ is optionally substituted cyclohexyl; or R₁₂ is optionally substituted 1-adamantyl providing that n is an integer of 1;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is CH;
or a pharmaceutically acceptable salt thereof.

6. A compound according to claim 4 wherein
R₁ is hydrogen;
R₂ is hydrogen or methyl;
R₃ is methyl;
R₄ is —(CHR₁₁)ₙR₁₂ in which
n is an integer of 1;
R₁₁ is hydrogen;
R₁₂ is optionally substituted 1-adamantyl;
W is optionally substituted aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₉ is hydrogen, optionally substituted alkyl, aralkyl, heteroaralkyl or alkanoyl;
Z is —C(O)R₆, —C(O)OR₆, —C(O)NR₆R₇, —C(S)NR₆R₇, —S(O)₂R₆, or —R₈ in which
R₈ is optionally substituted alkyl, aralkyl or heteroaralkyl;
—X═Y— is —CH₂—, oxygen or —NR₁₀— in which R₁₀ is hydrogen or methyl;
or a pharmaceutically acceptable salt thereof.

7. A compound according to claim 3 of the formula 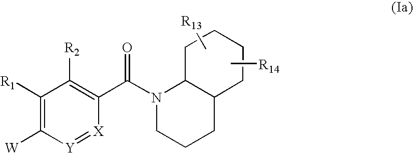
wherein
R₁ and R₂ are independently hydrogen, halo, optionally substituted amino, lower alkyl or lower alkoxy; or
R₁ and R₂ combined together with the carbon atoms to which they are attached form an optionally substituted 6-membered aromatic ring;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl; or
R₅ and R₁ are alkylene which combined together with the nitrogen atom to which R₅ is attached and the carbon atoms to which W and R₁ are attached form a 5-membered ring;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl; or
W is aryl or heteroaryl; or
W is hydrogen provided that R₁ is —NR₅Z in which Z is —C(O)R₆, —C(O)OR₆, —C(O)NR₆R₇, —C(S)NR₆R₇, —S(O)₂R₆, or —R₈; or
W and R₁ combined together with the carbon atoms they are attached to form a 6-membered aromatic ring optionally substituted with alkyl, alkoxy, aryl, heteroaryl, halo, —NR₅Z, —C(O)NR₆R₇, —OR₉ or —OC(O)NR₆R₇;
X is CH;
Y is CH or nitrogen; or
—X═Y— is —CH₂—, oxygen, sulfur or —NR₁₀— in which R₁₀ is hydrogen or methyl;
R₁₃ and R₁₄ are independently hydrogen, hydroxy or optionally substituted lower alkyl; or
a pharmaceutically acceptable salt thereof.

8. A compound according to claim 7 wherein
R₁ is hydrogen;
R₂ is hydrogen, chloro, methoxy, ethoxy, propoxy or optionally substituted amino;
W is —NR₅C(O)R₆, —NR₅C(O)OR₆, —NR₅C(O)NR₆R₇, —NR₅C(S)NR₆R₇, —NR₅S(O)₂R₆, —NR₅R, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl; or
a pharmaceutically acceptable salt thereof.

9. A compound according to claim 7 wherein
R₁ is methyl, methoxy or optionally substituted amino;
R₂ is hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
or a pharmaceutically acceptable salt thereof.

10. A compound according to claim 7 wherein
R₁ and R₂ are hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl; or
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is nitrogen;
R₁₃ and R₁₄ are independently hydrogen, hydroxy or optionally substituted lower alkyl;
or a pharmaceutically acceptable salt thereof.

11. A compound according to claim 7 wherein
W is hydrogen;
R₂ is hydrogen;
R₁ is —NR₅Z in which Z is —C(O)R₆, —C(O)OR₆, —C(O)NR₆R₇, —C(S)NR₆R₇, —S(O)₂R₆ or —R₈ in which
R₅ and R₇ are independently hydrogen or methyl;
R₆ is optionally substituted alkyd, aryl, hetroaryl, cycloalkyl, aralkyl or heteroaralkyl;
R₈ is optionally substituted alkyl, aralkyl or heteroaralkyl;
X is CH;
Y is CH;
R₁₃ and R₁₄ are independently hydrogen, hydroxy or optionally substituted lower alkyl;
or a pharmaceutically acceptable salt thereof.

12. A compound according to claim 7 of the formula 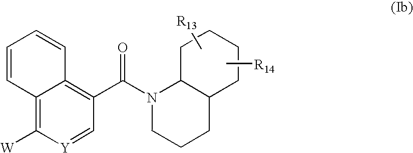
wherein
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
Y is CH;
R₁₃ and R₁₄ are independently hydrogen, hydroxy or optionally substituted lower alkyl;
or a pharmaceutically acceptable salt thereof.

13. A compound according to claim 7 of the formula 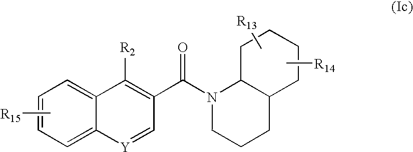
wherein
R₂ is hydrogen, halo or alkoxy;
Y is CH or nitrogen;
R₁₃ and R₁₄ are independently hydrogen, hydroxy or optionally substituted lower alkyl;
R₁₅ is hydrogen, —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
or a pharmaceutically acceptable salt thereof.

14. A compound according to claim 7 of the formula 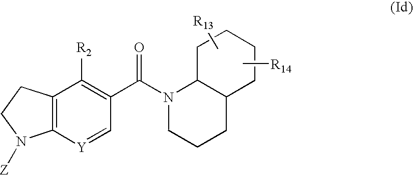
wherein
R₂ is hydrogen;
Z is —C(O)R₆, —C(O)OR₆, —C(O)NR₆R₇, —C(S)NR₆R₇, —S(O)₂R₆, or —R₈ in which
R₆ is optionally substituted alkyl, aryl, hetroaryl, cycloalkyl, aralkyl or heteroaralkyl;
R₇ is hydrogen or methyl;
R₈ is hydrogen, optionally substituted alkyl, aralkyl or heteroaralkyl;
Y is CH;
R₁₃ and R₁₄ are independently hydrogen, hydroxy or optionally substituted lower alkyl;
or a pharmaceutically acceptable salt thereof.

15. A compound according to claim 7 of the formula 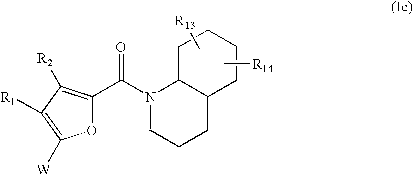
wherein
R₁ and R₂ are independently hydrogen, halo or lower alkyl;
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
Z is —C(O)R₆, —C(O)OR₆, —C(O)NR₆R₇, —C(S)NR₆R₇, —S(O)₂R₆, or —R₈;
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
R₁₃ and R₁₄ are independently hydrogen, hydroxy or optionally substituted lower alkyl;
or a pharmaceutically acceptable salt thereof.

16. A compound according to claim 7 of the formula 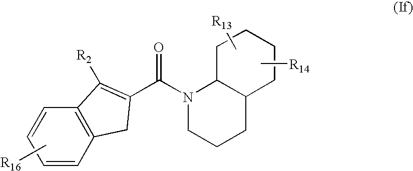
wherein
R₂ is hydrogen, halo or lower alkyl;
R₁₃ and R₁₄ are independently hydrogen, hydroxy or optionally substituted lower alkyl;
R₁₆ is hydrogen, halo, alkyl, aryl, heteroaryl or —NR₅Z in which
Z is —C(O)R₆, —C(O)OR₆, —C(O)NR₆R₇, —C(S)NR₆R₇, —S(O)₂R₆, or —R₈;
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
or a pharmaceutically acceptable salt thereof.

17. A compound according to claim 7 of the formula 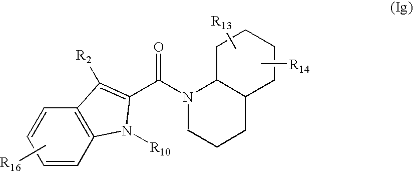
wherein
R₂ is hydrogen, halo or lower alkyl,
R₁₀ is hydrogen or methyl;
R₁₃ and R₁₄ are independently hydrogen, hydroxy or optionally substituted lower alkyl;
R₁₆ is hydrogen, halo, alkyl, aryl, heteroaryl or —NR₅Z in which
Z is —C(O)R₆, —C(O)OR₆, —C(O)NR₆R₇, —C(S)NR₆R₇, —S(O)₂R₆, or —R₈;
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
or a pharmaceutically acceptable salt thereof.

18. A compound according to claim 3 of the formula 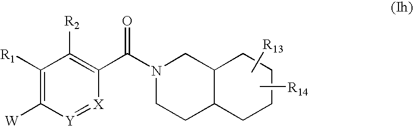
wherein
R₁ and R₂ are independently hydrogen, halo, optionally substituted amino, lower alkyl or lower alkoxy; or
R₁ and R₂ combined together form an optionally substituted 6-membered aromatic ring;
W is —NR₅C(O)R₆, NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl; or
R₅ and R₁ are alkylene which combined together with the nitrogen atom to which R₅ is attached and the carbon atoms to which W and R₁ are attached form a 5-membered ring;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl; or
W is aryl or heteroaryl; or
W and R₁ combined together with the carbon atoms to which they are attached form a 6-membered aromatic ring optionally substituted with alkyl, alkoxy, aryl, heteroaryl, halo, —NR₅Z, —C(O)NR₆R₇, —OR₉ or —OC(O)NR₆R₇ in which
Z is —C(O)R₆, —C(O)OR₆, —C(O)NR₆R₇, —C(S)NR₆R₇, —S(O)₂R₆, or —R₈;
R₁₃ and R₁₄ are independently hydrogen, hydroxy or optionally substituted lower alkyl;
X is CH;
Y is CH or nitrogen; or
—X═Y— is —CH₂—, oxygen, sulfur or —NR₁₀— in which R₁₀ is hydrogen or methyl;
or a pharmaceutically acceptable salt thereof.

19. A compound according to claim 18 wherein
R₁ is hydrogen;
R₂ is hydrogen, chloro, methoxy, ethoxy, propoxy or optionally substituted amino;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(C)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
or a pharmaceutically acceptable salt thereof.

20. A compound according to claim 18 wherein
R₁ is methyl, methoxy or optionally substituted amino;
R₂ is hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is CH;
R₁₃ and R₁₄ are independently hydrogen, hydroxy or optionally substituted lower alkyl;
or a pharmaceutically acceptable salt thereof.

21. A compound according to claim 18 wherein
R₁ and R₂ are hydrogen;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
X is CH;
Y is nitrogen;
R₁₃ and R₁₄ are independently hydrogen, hydroxy or optionally substituted lower alkyl;
or a pharmaceutically acceptable salt thereof.

22. A compound according to claim 18 of the formula 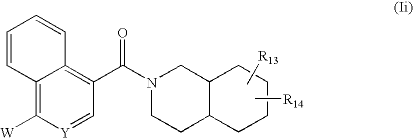
wherein
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is hydrogen, optionally substituted alkyl, aralkyl, heteroaralkyl or alkanoyl;
Y is CH;
R₁₃ and R₁₄ are independently hydrogen, hydroxy or optionally substituted lower alkyl;
or a pharmaceutically acceptable salt thereof.

23. A compound according to claim 18 of the formula 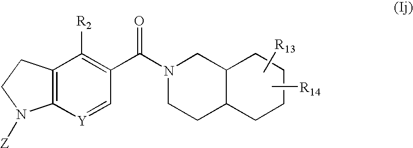
wherein
R₂ is hydrogen;
Z is —C(O)R₆, —C(O)OR₆, —C(O)NR₆R₇, —C(S)NR₆R₇, —S(O)₂R₆, or —R₈ in which
R₆ is optionally substituted-alkyl, aryl, hetroaryl, cycloalkyl, aralkyl or heteroaralkyl;
R₇ is hydrogen or methyl;
R₈ is hydrogen, optionally substituted alkyl, aralkyl or heteroaralkyl;
Y is CH;
R₁₃ and R₁₄ are independently hydrogen, hydroxy or optionally substituted lower alkyl;
or a pharmaceutically acceptable salt thereof.

24. A compound according to claim 3 of the formula 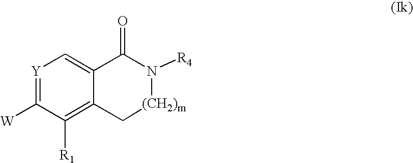
wherein
R₁ is hydrogen;
R₄ is —(CHR₁₁)ₙR₁₂ in which
n is zero or an integer from 1 to 2;
R₁₁ is hydrogen;
R₁₂ is aryl, heteroaryl, heterocyclyl or cycloalkyl;
W is —NR₅C(O)R₆, —NR₅C(O)OR₆, —NR₅C(O)NR₆R₇, —NR₅C(S)NR₆R₇, —NR₅S(O)₂R₆, —NR₅R₈, —C(O)NR₆R₇, —OR₉ or —OC(O)NR₆R₇ in which
R₅ and R₇ are independently hydrogen or methyl;
R₆ is optionally substituted alkyl, aryl, hetroaryl, cycloalkyl, aralkyl or heteroaralkyl;
R₈ is optionally substituted alkyl, aralkyl or heteroaralkyl;
R₉ is (C₁₋₆)alkyl substituted by cycloalkyl, alkoxy, cycloalkoxy, alkylthio, aryloxy, heterocyclooxy, arylthio, aryl or heteroaryl;
Y is CH;
m is zero or an integer from 1 to 2;
or a pharmaceutically acceptable salt thereof.

25. A method for the inhibition of 11β-hydroxysteroid dehydrogenase type 1 (11β-HSD1) oxoreductase activity in mammals, which method comprises administering to a mammal in need thereof a therapeutically effective amount of a compound of claim 1.

26. A method to control glucocorticoid concentration in mammals which method comprises administering to a mammal in need thereof a therapeutically effective amount of a compound of claim 1.

27. A method according to claim 26, which comprises lowering intracellular and hepatic glucocorticoid concentrations, increasing insulin sensitivity in the adipose tissue and in the muscle, reducing lipolysis and free fatty acid production in the adipose tissue, and inhibiting hepatic gluconeogenesis.

28. A method for the treatment of conditions associated with 11β-HSD1 oxoreductase activity in mammals which comprises administering to a mammal in need thereof a therapeutically effective amount of a compound of claim 1.

29. A method for the treatment of glucocorticoid associated disorders in mammals which method comprises administering to a mammal in need thereof a therapeutivally effective amount of a compound of claim 1.

30. A method according to claim 29, which comprises administering a compound of claim 1 in combination with a therapeutically effective amount of insulin, insulin derivative or mimetic, insulin secretagogue, insulinotropic sulfonylurea receptor ligand, insulin sensitizer, biguanide, alpha-glucosidase inhibitor, GLP-1, GLP-1 analog or mimetic, DPP-IV inhibitor, hypolipidemic agent, anti-obesity agent, cholestyramine, fibrate, nicotinic acid, or aspirin.

31. A method for the treatment of impaired glucose tolerance in Type 2 diabetes which method comprises administering to a mammal in need thereof a therapeutically effective amount of a compound of claim 1.

32. A method for the treatment of Syndrome-X, dyslipidemia, hypertension and central obesity which method comprises administering to a mammal in need thereof a therapeutically effective amount of a compound of claim 1.

33. A pharmaceutical composition comprising a compound of claim 1 preferably in a therapeutically effective amount, in combination with one or more pharmaceutically acceptable carriers.

34. A pharmaceutical composition comprising a compound according to claim 1 preferably in a therapeutically effective amount, in combination with insulin, insulin derivative or mimetic, insulin secretagogue, insulinotropic sulfonylurea receptor ligand, insulin sensitizer, biguanide, alpha-glucosidase inhibitor, GLP-1, GLP-1 analog or mimetic, DPP-IV inhibitor, hypolipidemic agent, anti-obesity agent, cholestyramine, fibrate, nicotinic acid, or aspirin, preferably in a therapeutically effective amount.

35. A pharmaceutical composition according to claim 33, for the treatment of impaired glucose tolerance, Type 2 diabetes and central obesity.

36. Use of a pharmaceutical composition according to claim 33, for the preparation of a medicament for the treatment of conditions associated with 11β-HSD1 oxoreductase activity.

37. A compound according to claim 1, for use as a medicament.

38. Use of a compound according to claim 1, for the preparation of a pharmaceutical composition for the treatment of conditions associated with 11β-HSD1 oxoreductase activity.

39. Use according to claim 36, wherein the condition associated with 11β-HSD1 oxoreductase activity is selected from impaired glucose tolerance, Type 2 diabetes, insulin resistance, dyslipidemia, metabolic Syndrome X and central obesity.
